(19) United States
(12) Patent Application Publication
(10) Pub. No.: US 2022/0228188 A1
(43) Pub. Date: Jul. 21, 2022

(54) DEVICES AND METHODS FOR PEPTIDE SAMPLE PREPARATION

(71) Applicant: Quantum-Si Incorporated, Guilford, CT (US)

(72) Inventors: Michele Millham, Guilford, CT (US); Jonathan C. Schultz, Guilford, CT (US); Omer Ad, Madison, CT (US); Caixia Lv, Guilford, CT (US)

(73) Assignee: Quantum-Si Incorporated, Guilford, CT (US)

(21) Appl. No.: 17/579,354

(22) Filed: Jan. 19, 2022

Related U.S. Application Data
(60) Provisional application No. 63/139,332, filed on Jan. 20, 2021.

(51) Int. Cl. C12P 21/06; B01L 3/00; B01L 7/00

(52) U.S. Cl. CPC C12P 21/06; B01L 3/5027; B01L 2200/0689; B01L 2300/18; B01L 2300/123; B01L 7/00

(57) ABSTRACT

Aspects of this disclosure related to methods, articles, kits, and/or systems for the preparation and/or study of one or more target molecules in a sample. In some embodiments, a target molecule is a peptide, a protein, or a fragment or derivative thereof. Through the use of methods, articles, kits, and/or systems of the instant disclosure, target molecules may, in some embodiment, be more readily sequenced or prepared for sequencing.

### RELATED APPLICATIONS

This application claims priority under 35 U.S.C. § 119(e) to U.S. Provisional Patent Application No. 63/139,332, filed Jan. 20, 2021, entitled “Devices and Methods for Peptide Sample Preparation,” which is incorporated herein by reference in its entirety for all purposes.

### TECHNICAL FIELD

Methods, articles, systems, and kits related to manipulation and/or preparation of biomolecules such as peptides are generally described.

### BACKGROUND

Proteomics has emerged as important in the study of biological systems. These analyses of an individual organism or sample type can provide insights into cellular processes and response patterns, which lead to improved diagnostic and therapeutic strategies. The complexity surrounding protein compositions and modification present challenges in determining large-scale sequencing information for a biological sample.

Improved and more convenient techniques and systems for manipulating (e.g., preparing) protein compositions are desirable.

### SUMMARY

Aspects of this disclosure related to methods, articles, kits, and/or systems for the preparation and/or study of one or more target molecules in a sample. In some embodiments, a target molecule is a peptide, a protein, or a fragment or derivative thereof. Through the use of methods, articles, kits, and/or systems of the instant disclosure, target molecules may, in some embodiment, be more readily sequenced or prepared for sequencing. The subject matter of the present invention involves, in some cases, interrelated products, alternative solutions to a particular problem, and/or a plurality of different uses of one or more systems and/or articles.

In one aspect, a fluidic device for preparing a peptide sample is described. In some embodiments, the fluidic device for preparing a peptide sample comprises: a derivatization agent reservoir configured to receive a derivatization agent capable of derivatizing an amino acid side chain; and a quenching region fluidically connected to the derivatization agent reservoir via one or more microchannels, wherein the quenching region comprises a solid substrate having a surface comprising functional groups that are capable of reacting with the derivatization agent.

In some embodiments, the fluidic device for preparing a peptide sample comprises: an incubation region configured to facilitate heating of a sample, the incubation region comprising an incubation channel, wherein the incubation channel is a microchannel; a derivatization region; and a derivatization agent reservoir configured to receive a derivatization agent capable of derivatizing an amino acid side chain, wherein the derivatization agent reservoir is fluidically connected to the incubation channel and the derivatization region such that a fluid can be transported from the incubation channel, through the derivatization agent reservoir, and to the derivatization region.

In another aspect, a kit for preparing a peptide sample is described. In some embodiments, the kit for preparing a peptide sample comprises a fluidic device comprising an incubation region comprising an incubation channel, wherein the incubation channel is a microchannel; and one or more reagents chosen from: a reducing agent, an amino acid side chain capping agent, and a protein digestion agent; wherein the incubation region is configured to receive the one or more reagents.

In another aspect, a method for preparing a peptide sample is described. In some embodiments, the method for preparing a peptide sample comprises: incubating a peptide sample in an incubation region of a fluidic device, the fluidic device comprising at least one microchannel, to form a digested peptide sample, the peptide sample comprising a mixture comprising: a protein, a reducing agent, an amino acid side chain capping agent, and a protein digestion agent; wherein during the incubating: the reducing agent reduces an amino acid side chain of the protein to form a reduced amino acid side chain, the amino acid side chain capping agent forms a covalent bond with the reduced amino acid side chain to form a capped amino acid side chain, and the protein digestion agent induces proteolysis of the protein comprising the capped amino acid side chain to form one or more capped peptides, thereby forming the digested peptide sample.

In some embodiments, the method for preparing a peptide sample comprises: incubating a peptide sample in an incubation region of a first fluidic device portion, the fluidic device portion comprising one or more microchannels, to form a digested peptide sample; and functionalizing one or more peptides of the digested peptide sample to form a functionalized peptide sample, wherein the functionalizing step comprises: derivatizing an amino acid side chain of the one or more peptides using a derivatization agent in a derivatization region of a second fluidic device portion to form an unquenched mixture comprising one or more derivatized peptides and excess derivatization agent, and quenching the unquenched mixture to form a quenched mixture by removing at least some of the excess derivatization agent in a quenching region of a third fluidic device portion.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention are described by way of example with reference to the accompanying figures, which are schematic and are not intended to be drawn to scale unless otherwise indicated. In some embodiments of the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1D show schematic illustrations of fluidic devices comprising incubation regions, incubation channels, derivatization regions, derivatization agent reservoirs, and derivatization reagent reservoirs, according to certain embodiments;

FIGS. 2A-2B show schematic illustrations of fluidic devices comprising derivatization agent reservoirs and quenching regions, according to certain embodiments;

FIGS. 3A-3B show exemplary workflows for digestion of peptide samples, according to certain embodiments;

FIG. 4 shows a peptide connected to an immobilization complex, according to certain embodiments;

FIG. 5 shows a reaction scheme for the preparation of an immobilization complex, according to certain embodiments;

FIG. 6 shows a process for immobilizing a peptide for sequencing, according to certain embodiments;

FIG. 7 shows a derivatization reaction scheme, according to certain embodiments;

FIG. 8 shows a scheme of peptide immobilization, according to certain embodiments;

FIG. 9 shows a schematic illustration of a system comprising a sample preparation module and a detection module, according to certain embodiments;

FIGS. 10A-10B show schematic illustrations of arrangements of fluidic device portions, according to certain embodiments;

FIG. 11 shows a cross-sectional schematic illustration of channels of a fluidic device, according to certain embodiments;

FIGS. 12A-12B show sample preparation devices comprising multiple fluidic devices, according to certain embodiments;

FIGS. 13A-13B show processes for digesting and preparing proteins, according to certain embodiments;

FIGS. 14A-14I show schematic illustrations of fluidic devices comprising incubation regions, derivatization regions, and quenching regions, according to certain embodiments;

FIGS. 15A-15D show the results of sequencing peptide samples prepared in an exemplary fluidic device, according to certain embodiments;

FIGS. 16A-16B shows schematic illustrations of a fluidic device portion comprising an incubation region, according to certain embodiments; and

FIGS. 17A-17B shows schematic illustrations of a fluidic device portion comprising a derivatization region, according to certain embodiments.

### DETAILED DESCRIPTION

In some aspects, the disclosure provides methods, articles, systems, and kits for the preparation and analysis of peptide samples (e.g. peptide libraries) (e.g., using fluidic devices). Some such embodiments may accelerate preparation and analysis of peptide samples (e.g., for peptide/protein sequencing). In some embodiments, methods, articles, and kits described herein facilitate the incubation, digestion, functionalization (e.g. via derivatization), quenching (e.g., via contact with functionalized solid substrates), and/or purification of peptide samples within portions of fluidic devices. In some instances, the portions of fluidic devices may be connected to one another (e.g., as part of a same cartridge). These embodiments may provide advantages for the preparation and analysis of peptide samples. For example, these embodiments may permit two or more steps of the preparation and analysis of peptide samples to be performed automatedly and sequentially (and in some instances simultaneously), without the need for intervening actions such as cleaning or separation of mixed compounds that would otherwise require direct human involvement. In some embodiments, peptide samples comprise proteins or peptides, and analysis of the peptide samples may permit sequencing of the proteins or peptides.

In some embodiments, fluidic devices (e.g., cartridges) configured for peptide preparation are provided. Some such configurations may include, for example, regions (e.g., reservoirs and/or channels) adapted for, and in some instances including, reagents for chemically modifying peptides (e.g., for digestion/fragmentation, derivatization, or a combination thereof). The fluidic devices may comprise one or more microchannels. In some embodiments, fluidic devices comprise incubation regions configured to facilitate digestion and/or other modifications such as conjugation of peptides (e.g., by including serpentine microchannels and/or thermally conductive solid materials). Multiple chemical modifications for digestion of peptides may occur automatedly in the fluidic device (e.g., by sending a mixture of multiple reagents such as reducing agents, capping agents, and/or digestion agents to the incubation channel). The fluidic devices (e.g., cartridges) may include a derivatization region and derivatization agent or reagent reservoirs (e.g., as part of a functionalization process), and may also include a quenching region to facilitate removal and/or deactivation of excess reagents. The fluidic devices (e.g., microfluidic cartridges) may be configured to operatively couple to a system comprising a sample preparation module (e.g., comprising a peristaltic pump) and a detection module (e.g., a peptide sequencing module).

Workflows for the preparation (and in some instances, analysis) of protein samples often require several steps. Since each step may ordinarily be associated with a degree of material loss, inefficiency, and time expenditure, approaches that eliminate or automate some or all of the steps of the workflow provide numerous benefits. However, eliminating or automating these steps is not a straightforward process, since the presence of chemical impurities or defects can also produce unexpected and detrimental effects.

One approach for simplifying workflows for the preparation (and in some instances, analysis) of peptide samples is the performance of one or more steps using fluidic devices (e.g., microfluidic devices). Such devices may offer exceptional control of chemical processes for preparation of the peptide sample, increasing reliability and yield. However, when using certain existing fluidic devices, it is still often necessary to perform some steps by hand. In the context of the present disclosure, the inventors have recognized a need to automate steps of the preparation and analysis of peptide samples, and have provided inventive solutions to meet this need.

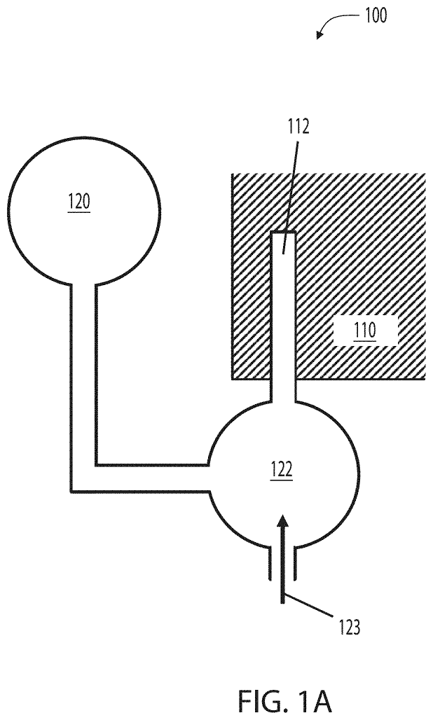

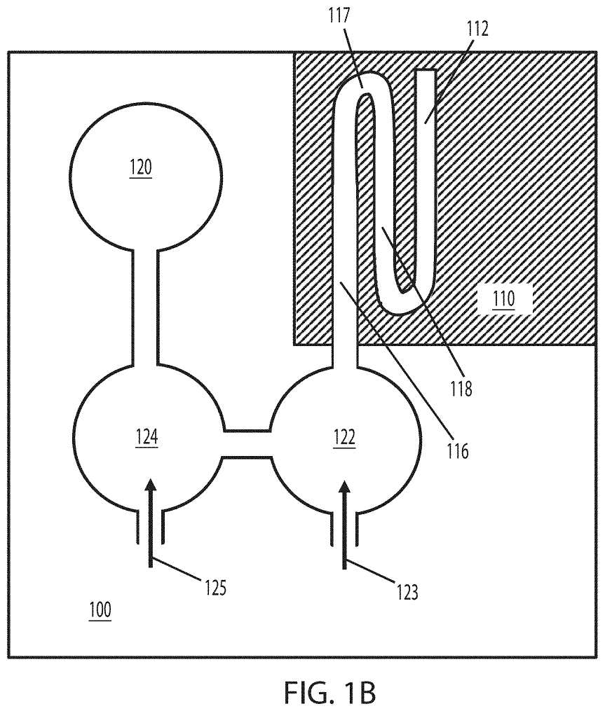

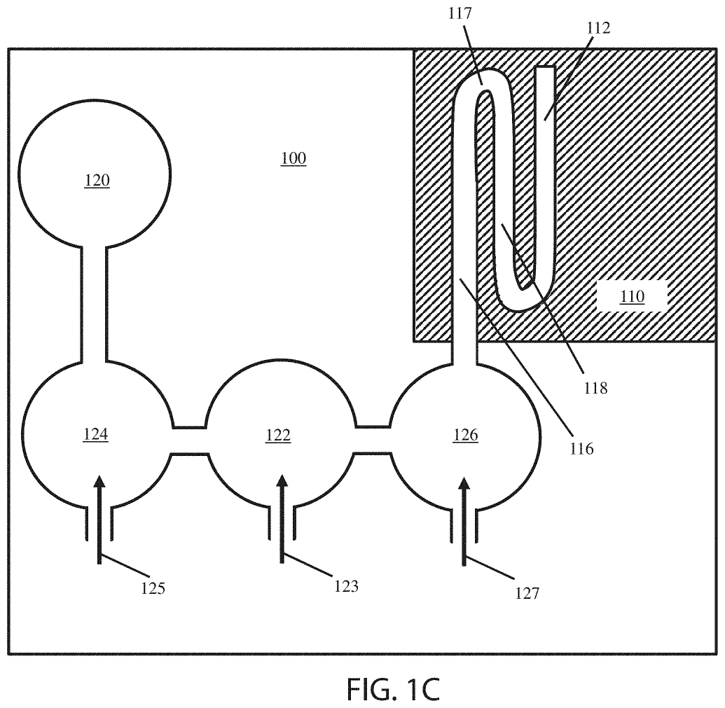

In one aspect, a method for preparing a peptide sample is disclosed. In some embodiments, the method comprises incubating a peptide sample in an incubation region of a fluidic device. In some embodiments, the incubation region is part of a first fluidic device portion. An incubation region may be configured to facilitate heating of a sample. In some embodiments, the incubation region comprises an incubation channel. The peptide sample may be incubated in an incubation channel of incubation region. In some embodiments, incubating a peptide sample forms a digested peptide sample. For example, FIGS. 1A-1D present a schematic illustrations of fluidic devices 100 comprising incubation region 110. In some embodiments, the peptide sample is incubated in incubation channel 112 of incubation region 110. Details of potentially suitable incubation conditions are described in more detail below.

In another aspect, a method comprises functionalizing one or more peptides of a digested peptide sample. Functionalizing peptides of a digested peptide sample may form a functionalized peptide sample. In some cases, functionalizing comprises derivatizing an amino acid side chain of the one or more peptides using a derivatization agent. For example, the digested peptides may be exposed to a derivatization agent (e.g., by dissolving derivatization agent in a solution comprising the peptides, mixing a solution comprising the peptides and a solution comprising the derivatization agent, etc.). In some embodiments, a derivatization agent is capable of derivatizing an amino acid side chain. An amino acid side chain may be derivatized in a derivatization region of a second fluidic device portion. For example, in FIG. 1A, an amino acid side chain of one or more peptides may be derivatized in derivatization region 120. Derivatizing an amino acid may form an unquenched mixture. In some cases, an unquenched mixture comprises one or more derivatized peptides and excess derivatization agent. Some embodiments comprise automatedly transporting at least some of the unquenched mixture from the derivatization region to a quenching region.

In this context of this disclosure, a process is generally considered to be automated if it is performed without direct human intervention during or between steps of the process. Often, an automated process is performed by computer-implemented controller that can perform steps of the process by following preprogrammed directions. The directions can be preprogrammed (e.g., by a manufacturer or a user), submitted manually by a user during the process, or a combination of the two. A human user interfacing with a computer-implemented controller is not considered direct human intervention in this context. A user manually introducing, removing, mixing, or transporting reagents and/or sample components (e.g., by pipetting/syringing, pouring, etc.) are examples of direct human intervention.

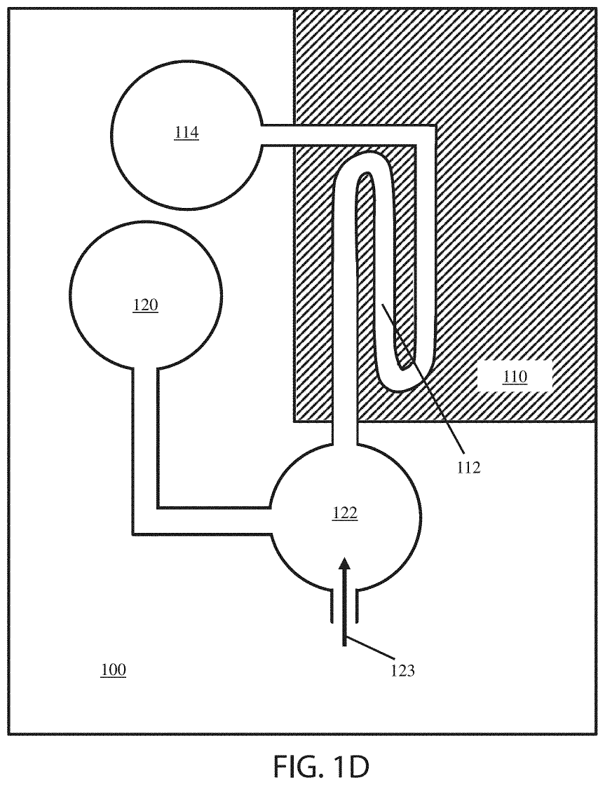

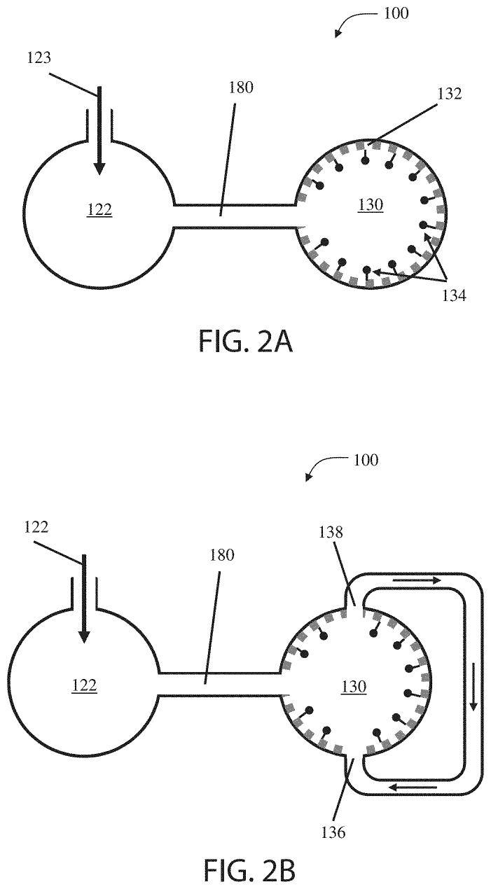

In some embodiments, a method further comprises quenching an unquenched mixture to form a quenched mixture. Quenching the unquenched mixture may remove at least some (e.g., at least 10 wt %, at least 25 wt %, at least 50 wt %, at least 75 wt %, at least 90 wt %, at least 95 wt %, at least 99 wt %, or more) of the excess derivatization agent in a quenching region. The quenching region may be part of, for example, a third fluidic device portion. The quenching region may comprise a solid substrate. The solid substrate may have surface comprising functional groups. The functional groups may be capable of reacting with the derivatization agent. For example, FIGS. 2A-2B illustrate fluidic device portions comprising quenching region 130, in accordance with some embodiments. Solid substrate surface 132 of quenching region 130 comprises functional groups (e.g., amines) 134 capable of reacting with a derivatization agent. In some embodiments, at least some of the digested peptide sample from incubation region is automatedly transported from incubation region to the derivatization region. In some embodiments, the functional groups may immobilize the derivatization agent, e.g. by reacting (for instance, by forming one or more covalent bonds, electrostatic interactions, and/or hydrogen bonds).

In another aspect, a fluidic device for preparing a peptide sample is described. In some embodiments, the fluidic device comprises a derivatization agent reservoir. The derivatization agent reservoir may be configured to receive a derivatization agent. In some embodiments, the fluidic device further comprises a quenching region. The quenching region may be fluidically connected to the derivatization agent reservoir. In some embodiments, the quenching region is connected to the derivatization agent reservoir via one or more channels. Some or all of these channels may be microchannels. For example, FIG. 2A illustrates a fluidic device for preparing a peptide sample that comprises derivatization agent reservoir 122 and quenching region 130, wherein quenching region 130 is connected to derivatization agent reservoir 122 by channel (e.g., microchannel) 180.

Another aspect of the present disclosure is a fluidic device for preparing a peptide sample comprising a derivatization region and a derivatization agent reservoir. In some embodiments, the derivatization region is configured to receive a derivatization agent. In some embodiments, the derivatization region comprises the derivatization agent (e.g., at least some of the derivatization agent is contained within the derivatization region). For example, in FIG. 1A, fluidic device 100 comprises derivatization region 120 and derivatization agent reservoir 122, and further comprises incubation region 110 that comprises incubation channel 112. In this embodiment, derivatization agent reservoir 122 is configured to receive derivatization agent 123 (e.g., prior to and/or during a peptide preparation process). In some embodiments, the fluidic device further comprises an incubation region comprising an incubation channel. In some cases, the derivatization agent reservoir is fluidically connected to the incubation channel and the derivatization region such that a fluid (e.g., comprising a peptide sample) can be transported from the incubation channel, through the derivatization agent reservoir, and to the derivatization region. For example, in FIG. 1A, derivatization agent reservoir 122 is fluidically connected to incubation channel 112 and derivatization region 120 such that a fluid can be transported from incubation channel 112, through derivatization agent reservoir 122, and to derivatization region 120.

In some embodiments, the fluidic device further comprises a derivatization reagent reservoir. In some embodiments, the derivatization reagent reservoir is configured to receive a derivatization reagent. For example, FIG. 1B presents a schematic illustration of fluidic device 100 comprising derivatization reagent reservoir 124 configured to receive derivatization reagent 125. In some embodiments, the derivatization reagent is capable of facilitating a reaction between the derivatization agent and the amino acid side chain. For example, the derivatization reagent may be a catalyst for the derivatization reaction. As a specific example, the derivatization agent may comprise an azide transfer reagent such as imidazole-1-sufonyl azide, and the derivatization reagent may be a source of Cu²⁺ such as copper sulfate. In some embodiments, the derivatization agent reservoir and the derivatization reagent reservoir are fluidically connected, such that a fluid can be transported from the incubation region (e.g. incubation channel), through the derivatization agent reservoir and the derivatization reagent reservoir, and to the derivatization region. For example, in FIG. 1B, fluid can be transported from incubation region 110, through derivatization agent reservoir 122, through derivatization reagent reservoir 124, and to derivatization region 120. In some embodiments, the fluid can be transported, in order, from the incubation region (e.g. incubation channel), through the derivatization agent reservoir and the derivatization reagent reservoir, and to the derivatization region.

In some embodiments, a derivatization reagent reservoir is a first derivatization reagent reservoir, and the fluidic device further comprises a second derivatization reagent reservoir. For example, FIG. 1C presents a schematic illustration of fluidic device 100 comprising first derivatization reagent reservoir 124 and second derivatization reagent reservoir 126. In some embodiments, the second derivatization reagent reservoir is configured to receive a second derivatization reagent. In some embodiments, the second derivatization reagent is capable of facilitating a reaction between the derivatization agent and the amino acid side chain. For example, the second derivatization reagent may be a pH adjusting reagent such as potassium carbonate (K₂CO₃). In some embodiments, the derivatization agent reservoir, the first derivatization reagent reservoir, and the second derivatization reagent reservoir are fluidically connected, such that a fluid can be transported, in order, from the incubation region (e.g. incubation channel), through the second derivatization reagent reservoir, through the derivatization agent reservoir, through the first derivatization reagent reservoir, and to the derivatization region. For example, in FIG. 1C, fluid can be transported from incubation region 110, through second derivatization reagent reservoir 126 (which is configured to receive second derivatization reagent 127), through derivatization agent reservoir 122 (which is configured to receive derivatization agent 123), through first derivatization reagent reservoir 124 (which is configured to receive first derivatization reagent 125), and to derivatization region 120. In some embodiments, a first derivatization reagent (e.g., a source of Cu²⁺ such as copper sulfate) is not exposed (e.g., mixed) with a second derivatization reagent (e.g., a pH adjusting reagent such as a salt comprising a basic buffer such as K₂CO₃) until the first derivatization reagent and the second derivatization reagent are combined (e.g., mixed) with the digested peptide sample. Avoiding pre-mixture of the first derivatization reagent and the second derivatization reagent may be beneficial in some instances where the first derivatization reagent and the second derivatization can react in such a way that adversely affects efficient derivatization.

In another aspect, a kit for preparing a peptide sample is described. In some embodiments, the kit comprises the fluidic device. In some embodiments, the kit comprises one or more reagents. Reagents may include: a reducing agent, an amino acid side chain capping agent, and/or a protein digestion agent. In some embodiments, the kit comprises two or more reagents chosen from a reducing agent, an amino acid side chain capping agent, and a protein digestion agent. In some embodiments, the kit comprises each of a reducing agent, an amino acid side chain capping agent, and a protein digestion agent. In some cases, the fluidic device comprises an incubation region that is configured to receive one or more of the reagents. In some embodiments, the fluidic device and reagents are packaged individually. In some embodiments, two or more parts of a kit (e.g. fluidic device, reagents) are packaged together. In some embodiments, all kit components are packaged together.

Some embodiments described herein are directed towards peptide samples. In some embodiments, the peptide sample comprises one or more peptides (e.g., proteins). In some embodiments, the peptides are at least partially (or completely) dissolved in a liquid solution (e.g., an aqueous buffer). In some, but not necessarily all embodiments, a peptide sample comprises a mixture comprising: a protein, a reducing agent, an amino acid side chain capping agent, and/or a protein digestion agent. In some embodiments, a peptide sample comprises a mixture comprising: a protein, a reducing agent, an amino acid side chain capping agent, and a protein digestion agent.

Any suitable reducing agent may be used to reduce a protein within a peptide sample. In some embodiments, the reducing agent is suitable for reducing a disulfide-bond. In some embodiments, the reducing agent may reversibly reduce a disulfide bond. Suitable reversable reducing agents may comprise compounds such as dithiothreitol (DTT), β-mercaptoethanol (BME), and/or Glutathione (GSH). In some embodiments, the reducing agent may irreversibly reduce a disulfide bond. Suitable irreversible reducing agents may comprise compounds such as tris(2-carboxyethyl)phosphine (TCEP). In some specific embodiments, the reducing agent comprises tris(2-carboxyethyl)phosphine (TCEP).

Any suitable amino acid side chain capping agent may be used to cap amino acid side chains of a protein within a peptide sample. In some embodiments, the amino acid side chain capping agent prevents the formation of disulfide bonds. In some embodiments, the amino acid side chain capping agent prevents the amino acid side chain from undergoing further reactivity such as nucleophile/electrophile or redox reactivity. In some embodiments, the amino acid side chain capping agent is a cysteine capping agent. In some embodiments, the amino acid side chain capping agent is a sulfhydryl-reactive alkylating reagent (e.g. a cysteine alkylation agent). For instance, in some embodiments, the amino acid side chain capping agent comprises a haloacetamide (e.g. chloroacetamide, iodoacetamide) or a haloacetate/haloacetic acid (e.g., chloroacetate/chloroacetic acid, iodoacetate/iodoacetic acid). In some embodiments, the amino acid side chain capping agent is an aromatic benzyl halide. For example, the amino acid side chain capping agent may be an aromatic benzyl halide derivative based on a benzene aromatic group, a pyridine aromatic group, a pyrazine aromatic group, and the like. Other examples of suitable cysteine alkylating agents include 4-vinylpyridine, acrylamide, and methanethiosulfonate. In some embodiments, the amino acid side chain capping agent comprises iodoacetamide.

Any suitable protein digestion method may be used, and several are described in detail below. In some specific embodiments, a protein digestion reagent is an enzymatic protein digestion reagent. For example, in some embodiments, the protein digestion agent comprises a protease. In some embodiments, the protease comprises trypsin, Lys-C, Asp-N, and/or Glu-C. In some embodiments, the protease is trypsin.

In some embodiments described herein, peptide samples are buffered to maintain pH within particular ranges. For instance, in some embodiments, peptide samples are buffered to maintain pH greater than or equal to 6, greater than or equal to 7, greater than or equal to 8, greater than or equal to 9, greater than or equal to 10, and/or greater at room temperature. In some embodiments, peptide samples are buffered to maintain pH less than or equal to 11, less than or equal to 10, less than or equal to 9, less than or equal to 8, less than or equal to 7, and/or less at room temperature. Combinations of these ranges are possible. For example, in some embodiments, peptide samples are buffered to maintain a pH of between 6 and 9.

In some embodiments described herein, a peptide sample may be buffered to a first pH range for a first step, and buffered to a second pH range for a second step. For example, in some embodiments, a peptide sample is buffered to a pH of 6 to 9 during incubation, and is then buffered to a pH of between 10 and 11 for a derivatization step. In some embodiments, the peptide sample is buffered to a desirable pH range for three, for four, for five, for six, for seven, for eight, for nine, and/or for ten or more steps. For example, in some embodiments, a peptide sample is buffered to a pH of 6 to 9 during incubation, and is then buffered to a pH of between 10 and 11 for a derivatization step, before being buffered to a pH of 7-8 for an immobilization complex forming step and a purification step.

Peptide samples may be buffered with any buffers suitable to the desired pH range of a peptide sample. For instance, in some embodiments it may be desirable to maintain a pH of between 6 and 9 for a peptide sample. Exemplary buffers appropriate to such pH ranges may comprise: HEPES buffer, phosphate buffers (e.g. PBS), Tris, Bis-Tris, carbonate buffers (e.g. buffers comprising: carbonates, such as sodium or potassium carbonate; and/or bicarbonates, such as sodium bicarbonate), which may be used separately or in combination to stabilize pH within a desired range. In some embodiments, a buffer appropriate to such pH ranges comprises: HEPES buffer, phosphate buffers (e.g. PBS), and/or carbonate buffers (e.g. buffers comprising: carbonates, such as sodium or potassium carbonate; and/or bicarbonates, such as sodium bicarbonate). One of ordinary skill in the art would be familiar with these and many other buffer systems, and the use of un-listed buffer systems is contemplated here.

In some embodiments, a peptide sample comprises a biological sample. In some embodiments, a peptide sample comprises blood, saliva, sputum, feces, urine or buccal swab sample. In some embodiments, a biological sample is from a human, a non-human primate, a rodent, a dog, a cat, a horse, or any other mammal. In some embodiments, a biological sample is from a bacterial cell culture (e.g., an E. coli bacterial cell culture). A bacterial cell culture may comprise gram positive bacterial cells and/or gram negative bacterial cells. In some embodiments, a sample is a purified sample proteins that have been previously extracted. A blood sample may be a freshly drawn blood sample from a subject (e.g., a human subject) or a dried blood sample (e.g., preserved on solid media (e.g. Guthrie cards)). A blood sample may comprise whole blood, serum, plasma, red blood cells, and/or white blood cells.

In some embodiments, a peptide sample (e.g., a sample comprising cells or tissue), may be prepared, e.g., lysed (e.g., disrupted, degraded and/or otherwise digested) in a process in accordance with the instant disclosure. In some embodiments, a peptide sample to be prepared, e.g., lysed, comprises cultured cells, tissue samples from biopsies (e.g., tumor biopsies from a cancer patient, e.g., a human cancer patient), or any other clinical sample. In some embodiments, a peptide sample comprising cells or tissue is lysed using any one of known physical or chemical methodologies to release a target molecule (e.g., a target protein) from said cells or tissues. In some embodiments, a peptide sample may be lysed using an electrolytic method, an enzymatic method, a detergent-based method, and/or mechanical homogenization. In some embodiments, a peptide sample (e.g., complex tissues, gram positive or gram negative bacteria) may require multiple lysis methods performed in series. In some embodiments, if a peptide sample does not comprise cells or tissue (e.g., a peptide sample comprising purified protein), a lysis step may be omitted. In some embodiments, lysis of a peptide sample is performed to isolate target protein(s). In some embodiments, a lysis method further includes use of a mill to grind a peptide sample, sonication, surface acoustic waves (SAW), freeze-thaw cycles, heating, addition of detergents, addition of protein degradants (e.g., enzymes such as hydrolases or proteases), and/or addition of cell wall digesting enzymes (e.g., lysozyme or zymolase). Exemplary detergents (e.g., non-ionic detergents) for lysis include polyoxyethylene fatty alcohol ethers, polyoxyethylene alkylphenyl ethers, polyoxyethylene-polyoxypropylene block copolymers, polysorbates and alkylphenol ethoxylates, preferably nonylphenol ethoxylates, alkylglucosides and/or polyoxyethylene alkyl phenyl ethers. In some embodiments, lysis methods involve heating a peptide sample for at least 1-30 min, 1-25 min, 5-25 min, 5-20 min, 10-30 min, 5-10 min, 10-20 min, or at least 5 min at a desired temperature (e.g., at least 60° C., at least 70° C., at least 80° C., at least 90° C., or at least 95° C.).

In some embodiments, a peptide sample is prepared, e.g., lysed, in the presence of a buffer system. This buffer system may be used to make a slurry of the peptide sample, to suspend the peptide sample, and/or to stabilize the peptide sample during any known lysis methodology, including those methods described herein. In some embodiments, a peptide sample is prepared, e.g., lysed, in the presence of RIPA buffer, GCI buffer that comprises Guanidine-HCl buffer, Gly-NP40 buffer, a TRIS buffer, a HEPES buffer, or any other known buffering solution.

Many of the lysis methods described herein allow for the peptide sample to be lysed by mechanically homogenizing the peptide sample such that the cell walls of the peptide sample break down. For example, methods that cause lysis by mechanical homogenization include, but are not limited to bead-beating, heating (e.g., to high temperatures sufficient to disrupt cell walls, e.g., greater than 50° C., 60° C., 70° C., 80° C., 90° C., or 95° C.), syringe/needle/microchannel passage (to cause shearing), sonication, or maceration with a grinder. In some embodiments, any lysis methodology may be combined with any other lysis methodology. For example, any lysis methodology may be combined with heating and/or sonication and/or syringe/needle/microchannel passage to quicken the rate of lysis.

In some embodiments, peptide sample preparation comprises cell disruption (i.e., subsequent removal of unwanted cell and tissue elements following lysis). In some embodiments, cell disruption involves protein precipitation. In some embodiments, following precipitation, the lysed and disrupted peptide sample is subjected to centrifugation. In some embodiments, following centrifugation, the supernatant is discarded. Precipitation can be accomplished through multiple processes, including but not limited to those methods described in Winter, D. and H. Steen (2011). “Optimization of cell lysis and protein digestion protocols for the analysis of HeLa S3 cells by LC-MS/MS.” PROTEOMICS 11(24): 4726-4730. In some embodiments, proteins or peptides are immunoprecipitated. In some embodiments, centrifugation of precipitated proteins is followed by discarding of the supernatant and subsequent washing of the pellet fraction (e.g., washing using chloroform/methanol or trichloroacetic acid).

In some embodiments, a peptide sample (e.g., a peptide sample comprising a target protein) may be purified, e.g., following lysis, in a process in accordance with the instant disclosure. In some embodiments, a peptide sample may be purified using chromatography (e.g., affinity chromatography that selectively binds the peptide sample) or electrophoresis. In some embodiments, a peptide sample may be purified in the presence of precipitating agents. In some embodiments, after a purification step or method, a peptide sample may be washed and/or released from a purification matrix (e.g., affinity chromatography matrix) using an elution buffer. In some embodiments, a purification step or method may comprise the use of a reversibly switchable polymer, such as an electroactive polymer. In some embodiments, a peptide sample may be initially purified by electrophoretic passage of a peptide sample through a porous matrix (e.g., cellulose acetate, agarose, acrylamide).

In some embodiments, the target molecule(s) is fragmented/digested prior to enrichment. In some embodiments, the target molecule is fragmented/digested after enrichment. In some embodiments, the target molecule(s) is fragmented/digested without any enrichment of the target molecule(s).

In some embodiments, preparing a peptide sample comprises incubation (e.g., as part of an incubating step). The incubation step may be performed on a peptide sample comprising a mixture comprising each of a protein, a reducing agent, an amino acid side chain capping agent, and a protein digestion agent. In some embodiments, during incubation, the reducing agent reduces an amino acid side chain of the protein to form a reduced amino acid side chain. In some embodiments, (e.g., during a same incubation step), the amino acid side chain capping agent forms a covalent bond with reduced amino acid side chain to form a capped amino acid side chain. In some embodiments (e.g., during a same incubation step), the protein digestion agent induces proteolysis of the protein to form one or more peptides, thereby forming a digested peptide sample. The protein digestion agent may induce proteolysis of the protein comprising the capped amino acid side chain to form one or more capped peptides, thereby forming a digested peptide sample. It has been realized in the context of the present disclosure that certain existing peptide digestion techniques may involve performing some or all of the above-mentioned processes (e.g., reduction, capping, proteolysis) as separate steps (e.g., by introducing respective reagents in a stepwise manner). Surprisingly, it has been realized that satisfactory digestion can be achieved using a mixture that combines some or all of the reducing agent, the amino acid side chain capping agent, and the protein digestion agent with the peptide. Such a combination of steps and reagents may facilitate digestion of the peptide on a fluidic device (e.g., a cartridge comprising one or more microchannels) by simplifying the configuration and/or reducing a number of reservoirs and reagent inlets. Some or all of the above-mentioned processes (e.g., reduction, capping, proteolysis) may occur simultaneously or sequentially in the incubation region without direct human intervention (e.g., without intervening purification/workup steps, without manual transferring of reagents, without manual transferring of intermediate products). In some embodiments, some or all of the incubation step is performed automatedly.

In some embodiments, an incubating step (e.g., in an incubation region of a fluidic device) comprises maintaining the peptide sample at a temperature greater than or equal to 20° C., greater than or equal to 25° C., greater than or equal to 30° C., greater than or equal to 35° C., or greater than or equal to 37° C., or greater. In some embodiments, an incubating step comprises maintaining the peptide sample at a temperature less than or equal to 70° C., less than or equal to 50° C., less than or equal to 37° C., less than or equal to 35° C., or less than or equal to 30° C. Combinations of these ranges are possible. For example, an incubating step may comprise maintaining the peptide sample at a temperature greater than or equal to 20° C. and less than or equal to 70° C. In some embodiments, an incubating step comprises maintaining the peptide sample at a temperature within the above-mentioned ranges (e.g., 37° C.) for at least 1 minute, at least 2 minutes, at least 5 minutes, at least 10 minutes, at least 15 minutes, at least 20 minutes, at least 25 minutes, at least 30 minutes, at least 45 minutes, at least 1 hour, at least 2 hours, at least 3 hours, at least 4 hours, at least 5 hours, at least 6 hours, or greater. In some embodiments, an incubating step comprises maintaining the peptide sample at a temperature within the above-mentioned ranges (e.g., 37° C.) for less than or equal to 20 hours, less than or equal to 15 hours, less than or equal to 10 hours, or less. Combinations (e.g., maintaining an above-mentioned temperature for at least one minute and less than or equal to 20 hours, at least 6 hours and less than or equal to 10 hours) are possible.

Incubation may result in the digestion of a peptide sample. In general, digestion of a peptide sample can be conducted using any known method, but typically will involve a nonenzymatic or an enzymatic method. Approaches for nonenzymatic digestion include, but are not limited to, acid hydrolysis and/or cleavage using a digestion agent such as cyanogen bromide, hydroxylamine, iodosobenzoic acid, dimethyl sulfoxide-hydrochloric acid, BNPS-skatole [2-(2-nitrophenylsulfenyl)-3-methylindole], or 2-nitro-5-thiocyanobenzoic acid. Electro-physical digestion methods may be employed as well, including electrochemical oxidation and/or digestion in conjunction with microwaves.

Enzymatic methods of digestion typically utilize digestion agents such as proteases to fragment protein into component peptides. These enzymes include trypsin (which is typically favored for the size of the peptides generated and the generation of a basic residue at the carboxyl terminus of the peptide), chymotrypsin, LysC, LysN, AspN, GluC and/or ArgC. Enzymatic fragmentation/digestion methods may be selected and adjusted for ease of use, speed, automation and/or effectiveness. In some embodiments, enzymatic methods include enzyme immobilization on solid substrates. Enzymatic methods may be performed in flow (e.g., in a microfluidic channel). In some embodiments, enzymatic methods are performed in an incubation region. Digestion methods may be performed automatedly. Alternatively, or in addition, digestion methods may be performed manually. An enzymatic digestion may utilize any number or combination of enzymes and may further comprise any of the known nonenzymatic methods.

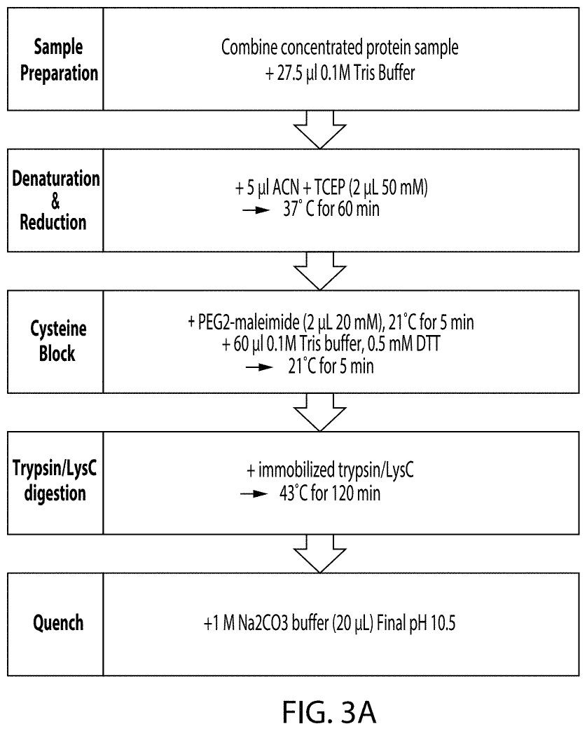

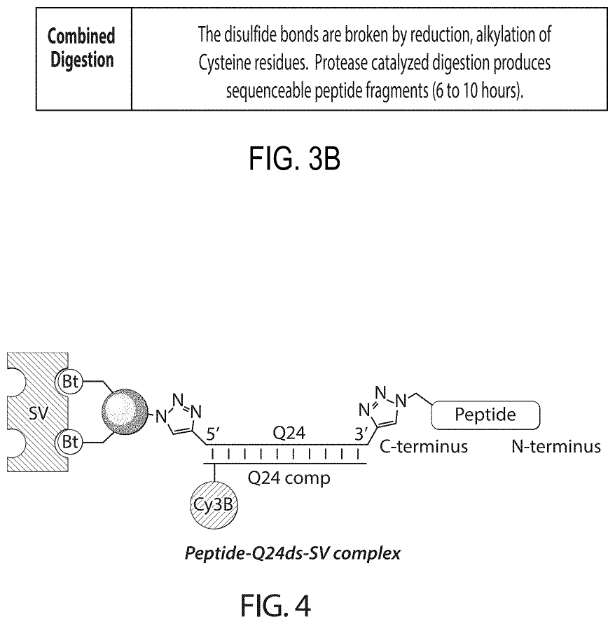

In some embodiments, a fragmentation/digestion process is as described in FIG. 3A. In some embodiments, a sample comprising target protein(s) is first denatured and reduced (e.g., using acetonitrile and TCEP). In some embodiments, target protein(s) to be fragmented are subjected to a cysteine block. In some embodiments, target protein(s) are fragmented using a mixture of trypsin and LysC (e.g., for 120 minutes). Enzymatic reactions may be quenched (e.g., using a quenching region of a fluidic device). In contrast, in some embodiments, a fragmentation/digestion process may be performed in a single step, wherein a peptide mixture comprising TCEP, iodoacetamide, and trypsin is incubated in an incubation region as described above. An exemplary embodiment of such a process is described in FIG. 3B.

Some embodiments comprise functionalizing one or more of the peptides of the digested peptide sample in a fluidic device to form a functionalized peptide sample. In some embodiments, functionalizing comprises derivatizing an amino acid side chain of the one or more peptides. In some embodiments, functionalizing comprises terminally functionalizing the one or more peptides (e.g., by one or more of the methods described below). In some embodiments, functionalizing one or more peptides of the digested peptide sample forms an unquenched mixture comprising one or more derivatized peptides. In some embodiments, a derivatization agent is used to derivatize an amino acid side chain (e.g. by one or more of the methods described below). The derivatization agent may comprise an azide transfer agent (e.g. imidazole-1-sulfonyl azide, trifluoromethanesulfonyl azide). For example, in some embodiments, the azide transfer agent comprises imidazole-1-sulfonyl azide. In some embodiments, the azide transfer agent comprises benzenesulfonyl-azide. An unquenched mixture comprising one or more derivatized peptides may also comprise excess derivatization agent. In some embodiments, functionalizing further comprises quenching an unquenched mixture to form a quenched mixture by removing at least some of the excess derivatization agent. Methods of quenching an unquenched mixture are described in more detail below.

Functionalization may further comprise conjugating one or more derivatized peptides to an immobilization complex to form one or more immobilization complex-conjugated peptides. Conjugation to an immobilization complex is described in detail below. However, in some specific embodiments, an immobilization complex may comprise DBCO, single-stranded DNA, and streptavidin (SV). For example, an immobilization complex may be DBCO-Q24-SV. At least some of the conjugating may be performed in an incubation region of a fluidic device. In some embodiments, the conjugating one or more derivatized peptides to immobilization complex may be performed in an immobilization complex forming region of a fourth fluidic device portion. Some embodiments may comprise automatedly transporting at least some of the quenched mixture from the quenching region to an immobilization complex-forming region. Some embodiments may comprise automatedly transporting at least some of the quenched mixture from the quenching region to an incubation region.

A target molecule may be functionalized at a terminal end or position. For example, a target protein may be functionalized at its N-terminal end or its C-terminal end.

### C-Terminal Carboxylate Functionalization

In one aspect, the present disclosure provides a method of selective C-terminal functionalization of a peptide, comprising:

a. reacting a plurality of peptides of Formula (I):

P—R(CO₂H)ₙ (I)

or salts thereof;
with a compound of Formula (II):

HX-L₁-R₁ (II)

to obtain a plurality of compounds of Formula (III):

P—R [symbol] CO—X-L₁-R₁]ₙ (III)

or salts thereof; and

b. reacting the plurality of compounds of Formula (III), or salts thereof, with a compound of Formula (IV):

R₂-L₂-Z (IV)

to obtain a plurality of compounds of Formula (V):

P—R [symbol] CO—X-L₁-Y-L₂-Z]ₙ (V)

or salts thereof; wherein m, n, P, R(CO₂H)ₙ, HX, X, L₁, L₂, R₁, R₂, Y and Z are defined as follows.

m is an integer of 1-25, inclusive. In certain embodiments, m is 1-10, inclusive. In certain embodiments, m is 5-10, inclusive. In certain embodiments, m is 1-5, inclusive. In certain embodiments, m is 1, 2, 3, 4, 5, 6, 7 8 9, 10, 11, 12, 13, 14, 15, 16, 17, 18, 19, 20, 21, 22, 23, 24, or 25.

n is 1 or 2. In certain embodiments, n is 1. In certain embodiments, n is 2.

Each P independently is a peptide. In certain embodiments, P has 2-100 amino acid residues. In certain embodiments, P has 2-30 amino acid residues.

Each R(CO₂H)ₙ independently is an amino acid residue having n carboxylate moieties. n is 1 or 2. In certain embodiments, n is 1. When n is 1, R(CO₂H)ₙ is lysine or arginine. In a particular embodiment, R(CO₂H)ₙ is lysine. In another particular embodiment, R(CO₂H)ₙ is arginine. In certain embodiments, n is 2. When n is 2, R(CO₂H)ₙ is glutamic acid or aspartic acid. In a particular embodiment, R(CO₂H)ₙ is glutamic acid. In another particular embodiment, R(CO₂H)ₙ is aspartic acid.

HX is nucleophilic moiety that is capable of being acylated, wherein H is a proton. X is one or more heteroatoms. In certain embodiments, X is O, S, or NH, or NO.

L₁ is a linker. In certain embodiments, L₁ is a substituted or unsubstituted aliphatic chain, wherein one or more carbon atoms are optionally, independently replaced by a heteroatom, an aryl, heteroaryl, cycloalkyl, or heterocyclyl moiety. In certain embodiments, L₁ is polyethylene glycol (PEG). In other embodiments, L₁ is a peptide, or an oligonucleotide. In certain embodiments, L₁ is less than 5 nm. In certain embodiments L₁ is less than 1 nm.

L₂ is a linker, or is absent. In certain embodiments, L₂ is absent. In certain embodiments, L₂ is a substituted or unsubstituted aliphatic chain, wherein one or more carbon atoms are optionally, independently replaced by a heteroatom, an aryl, heteroaryl, cycloalkyl, or heterocyclyl moiety. In certain embodiments, L₂ is polyethylene glycol (PEG). In other embodiments, L₂ is a peptide, or an oligonucleotide. In certain embodiments L₂ is between 5-20 nm, inclusive.

R₁ is a moiety comprising a click chemistry handle. In certain embodiments, R₁ is a moiety comprising an azide, tetrazine, nitrile oxide, alkyne or strained alkene. In certain embodiments, the alkyne is a primary alkyne. In certain embodiments, the alkyne is a cyclic (e.g., mono- or polycyclic) alkyne (e.g., diarylcyclooctyne or bicycle[6.1.0]nonyne). In certain embodiments, R₁ is a moiety comprising an azide. In certain embodiments, the strained alkene is trans-cyclooctene. In certain embodiments, the tetrazine comprises the structure:

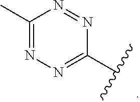 [SMILES: *c1nnc(C)nn1]

R₂ is a moiety comprising a click chemistry handle that is complementary to R₁. The click chemistry handle of R₂ is capable of undergoing a click reaction (i.e., an electrocyclic reaction to form a 5-membered heterocyclic ring) with R₁. For example, when R₁ comprises an azide, nitrile oxide, or a tetrazine, then R₂ may comprise an alkyne or a strained alkene. Conversely, when R₁ comprises an alkyne or a strained alkene, then R₂ may comprise an azide, nitrile oxide, or tetrazine. In certain embodiments, R₂ is a moiety comprising an azide, tetrazine, nitrile oxide, alkyne or strained alkene. In certain embodiments, the alkyne is a primary alkyne. In certain embodiments, the alkyne is a cyclic (e.g., mono- or polycyclic) alkyne (e.g., diarylcyclooctyne or bicycle[6.1.0]nonyne). In certain particular embodiments, R₂ comprises BCN. In other particular embodiments, R₂ comprises DBCO. In certain embodiments, the strained alkene is trans-cyclooctene. In certain embodiments, the tetrazine comprises the structure:

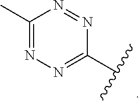 [SMILES: *c1nnc(C)nn1]

Y is a moiety resulting from the click reaction of R₁ and R₂. Y is a 5-membered heterocyclic ring resulting from an electrocyclic reaction (e.g., 3+2 cycloaddition, or 4+2 cycloaddition) between the reactive click chemistry handles of R₁ and R₂. In certain embodiments, Y is a diradical comprising a 1,2,3-triazolyl, 4,5-dihydro-1,2,3-triazolyl, isoxazolyl, 4,5-dihydroisoxazolyl, or 1,4-dihydropyridazyl moiety.

Z is a water-soluble moiety. In certain embodiments, Z imparts water-solubility to the compound to which it is attached. In certain embodiments, Z comprises polyethylene glycol (PEG). In certain embodiments, Z comprises single-stranded DNA. In certain embodiments (e.g., compounds of Formula (V)), Z further comprises biotin (e.g., bisbiotin). When Z comprises biotin (e.g., bisbiotin), Z may further comprise streptavidin. In certain embodiments, Z comprises double-stranded DNA. In some embodiments, the moieties of Z are capable of intermolecularly binding another molecule or surface, e.g., to anchor a compound comprising Z to the molecule or surface.

In certain embodiments, the compound of Formula (II) is of Formula (IIa):

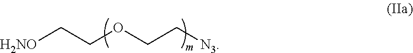

In certain embodiments, Formula (III) is of Formula (IIIa):

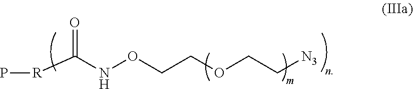

In certain embodiments, n is 1. In certain embodiments, n is 2. In certain embodiments, m is 1. In certain embodiments, m is 5.

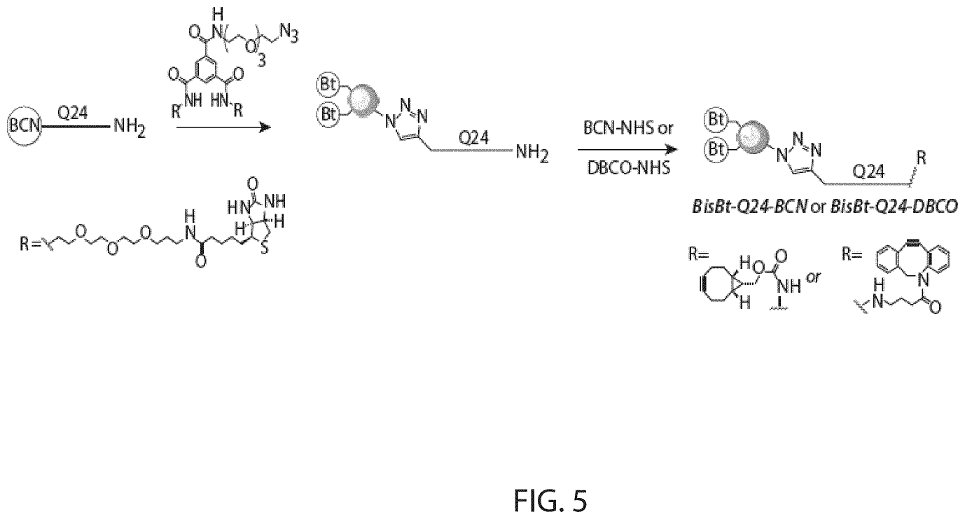

In certain embodiments, Formula (IV) comprises TCO, and single-stranded DNA. In certain embodiments, Formula (IV) further comprises biotin (e.g., bisbiotin). In certain embodiments, Formula (IV) is Q24-BisBt-BCN. In certain embodiments, Formula (IV) is Q24-BisBt-DBCO. In certain embodiments, Formula (IV) is Q24-BisBt-TCO. Generally, Formula (IV) may comprise a branching moiety (e.g., a 1, 3, 5-tricarboxylate moiety), wherein two branches are direct or indirect attachments to biotin moieties, and the third branch is an attachment to the water soluble moiety (e.g., a polynucleotide such as Q24). FIG. 4 presents an illustration of Q24-BisBt-BCN bonded to streptavidin. FIG. 5 presents a reaction scheme for the preparation of Q24-BisBt-BCN and/or Q24-BisBt-DBCO, according to certain embodiments. As shown in FIG. 4 and FIG. 5, in certain embodiments Formula (IV) comprises a triazole moiety derived from the click-coupling of fragments comprising (i) a bisbiotin-azide functionalized linker and (ii) an alkyne (e.g., BCN)-functionalized polynucleotide (e.g. Q24). The click-coupled product may be derivatized to introduce a further click handle R2, such as BCN or DBCO.

In certain embodiments, Formula (V) is of Formula (Va):

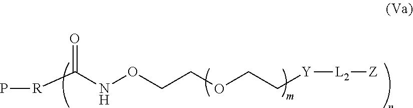

wherein m, n is 1 or 2; and L₂, Y, and Z are as defined above. In certain particular embodiments, n is 1. In certain particular embodiments, n is 2. In certain particular embodiments, m is 1. In certain particular embodiments, m is 5. In certain particular embodiments, L₂ is absent. In certain embodiments, Y comprises a moiety selected from 1,2,3-triazolyl, 4,5-dihydro-1,2,3-triazolyl, isoxazolyl, 4,5-dihydroisoxazolyl, and 1,4-dihydropyridazyl. In certain embodiments, Z comprises single-stranded DNA. In certain particular embodiments, Z comprises Q24. In certain embodiments, Z comprises double-stranded DNA. In certain embodiments, Z comprises double-stranded DNA. In certain embodiments, Z comprises biotin (e.g., bisbiotin). In certain embodiments, Z further comprises streptavidin.

In certain embodiments, the reaction of step (a) is performed in the presence of a carbodiimide reagent. In certain embodiments, the carbodiimide reagent is water soluble. In a particular embodiment, the carbodiimide reagent is 1-Ethyl-3-(3-dimethylaminopropyl)carbodiimide (EDC). In certain embodiments, the reaction of step (a) is performed at a pH in the range of 3-5. In certain embodiments (e.g., when to total peptide concentration below 1 mM), the concentration of EDC is about 10 mM and the concentration of the compound of Formula (II) is about 20 mM. In certain embodiments (e.g., in connection with Trypsin/LysC digestion, as described below) the concentration of the compound of Formula (II) is about may be about 50 mM and the concentration of EDC may be about 25 mM to suppress C-terminal intramolecular cyclization.

In certain embodiments of step (a), the plurality of compounds of Formula (III) is enriched prior to step (b), for example, by passing the compounds through a G10 sephadex column and/or passing the compounds through a C18 resin column. The use of C18 resin-based enrichment is particularly useful when the compound of Formula (II) is greater than about 200 g/mol. When G-10 sephadex is used in the enrichment, the elution buffer may be 0.5×PBS (pH 7.0). When C18 resin is used in the enrichment, the elution buffer may be 0.1% formic acid with 80% acetonitrile in water. The C18 eluent may be dried and the residue resuspended in 0.5×PBS prior to step (b).

In certain embodiments, the reaction of step (a) is performed in the presence of an immobilized carbodiimide reagent. For example, the carbodiimide reagent may be covalently attached to a moiety that is stationary and/or insoluble in the reaction solvent, thereby facilitating separation of excess reagent and/or reaction by-products and/or unreacted peptides. In certain embodiments, the immobilized carbodiimide reagent comprises a carbodiimide moiety that is covalently attached to a resin, such as polystyrene (PS). In certain embodiments, the PS-immobilized carbodiimide reagent is of the formula:

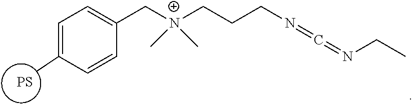

In certain embodiments, when the reaction of step (a) is performed in the presence of an immobilized carbodiimide reagent, for example, a PS-immobilized reagent as described herein, the reaction is performed at a pH in the range of 4 to 5 and/or at ambient temperature and or for about 20 minutes.

In certain embodiments, performing the reaction of step (a) in the presence of an immobilized carbodiimide reagent, for example, a PS-immobilized reagent as described herein, facilitates removal of all unreacted (i.e., non-acylated) peptides because the unreacted peptides remain covalently bound to the immobilized carbodiimide reagent.

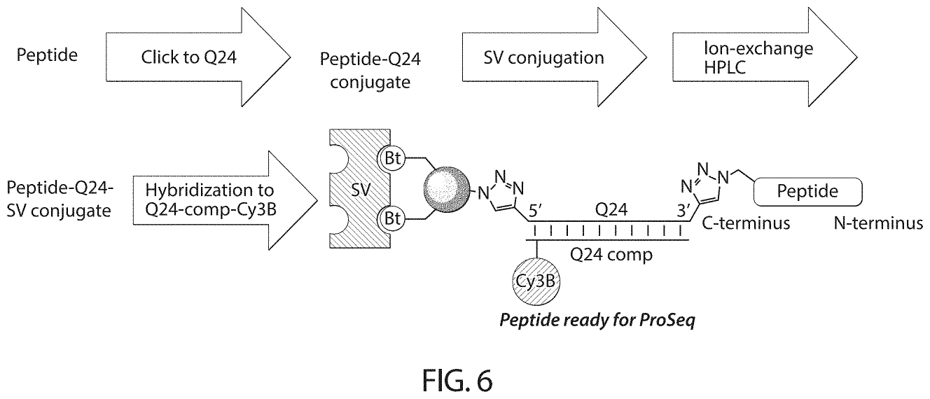

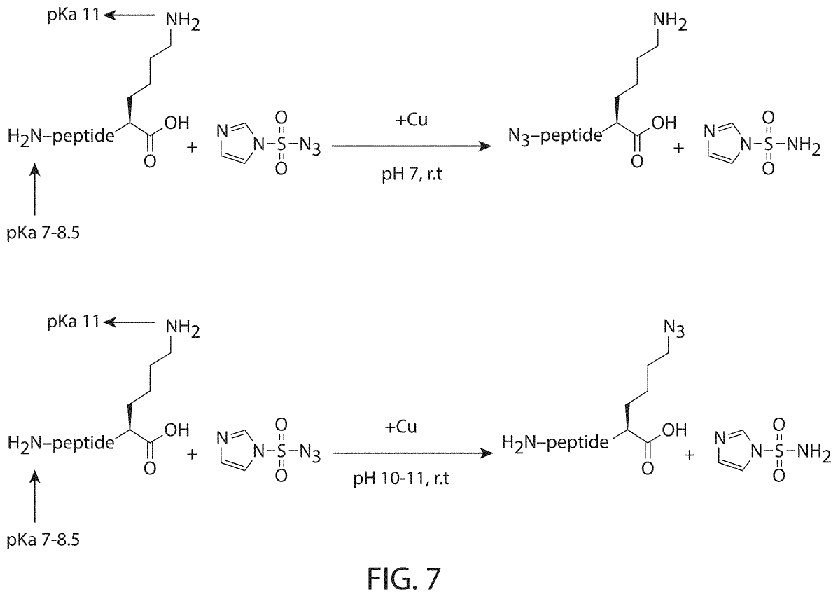

An exemplary process using an immobilized carbodiimide reagent is shown in FIG. 6. An exemplary flowchart for an automation compatible process is shown in FIG. 7. In certain embodiments of step (b), the click reaction between the plurality of compounds of Formula (III) and the compound of Formula (IV) is uncatalyzed. In certain embodiments, the click reaction is catalyzed, for example, using a copper salt (e.g., a Cu⁺ salt, or a Cu²⁺ salt that is reduced in situ to a Cu⁺ salt). Suitable Cu²⁺ salts include CuSO₄. In certain embodiments, the reaction of step (b) comprises heating the reaction mixture.

In certain embodiments, the compound of Formula (IV) is added to the plurality of compounds of Formula (III). In certain embodiments, the total concentration of the compound of Formula (IV) and the plurality of compounds of Formula (III) is maintained in the range between 10 μM to 1 mM.

In certain embodiments of step (b), when Z comprises single-stranded DNA, the method further comprises hybridizing a complementary DNA strand to the single-stranded DNA to obtain a compound wherein Z comprises double-stranded DNA. In certain embodiments, the single-stranded DNA is Q24 and the complementary DNA strand is a Cy3B-labeled Q24 complementary strand.

In certain embodiments of step (b), when Z comprises biotin (e.g., bisbiotin), the method further comprises contacting the biotin (e.g., bisbiotin) with streptavidin to obtain a compound wherein Z comprises biotin (e.g., bisbiotin) and streptavidin.

In certain embodiments, the plurality of peptides of Formula (I), or salts thereof, is obtained by subjecting a protein to enzymatic digestion to obtain a digestive mixture comprising the plurality of peptides of Formula (I), or salts thereof. In certain embodiments, the enzymatic digestion comprises cleaving the C-terminal bonds of aspartic acid and/or glutamic acid residues of the protein. In certain specific embodiments, the enzymatic digestion is Glu-C digestion.

In certain embodiments, the total concentration of the plurality of peptides of Formula (I), or salts thereof, after digestion of 20 μg protein is below 100 μM.

In certain embodiments, the enzymatic digestion is performed in phosphate buffer (pH 7.8) or ammonium bicarbonate buffer (pH 4.0).

In certain embodiments, the enzymatic digestion comprises cleaving the C-terminal bonds of lysine and/or arginine residues of the protein. In certain specific embodiments, the enzymatic digestion is Trypsin+Lys-C digestion.

In certain embodiments, the carboxylic acid moieties of the protein, if present, are protected prior to the enzymatic digestion. For example, the carboxylic acid moieties of the protein, if present, may be esterified prior to enzymatic digestion. In certain specific embodiments, the esterified carboxylic acids are methyl esters.

In certain embodiments, the sulfide moieties of the protein are protected prior to enzymatic digestion. In certain specific embodiments, the sulfide moieties are protected by exposing the protein to tris(carboxyethyl)phosphine (TCEP) and iodoacetamide (ICM), or maleimide.

In certain particular embodiments, TCEP is present in the form of a hydrochloride salt, i.e., TCEP.HCl.

In certain embodiments, the method further comprises the step of enriching the digestive mixture prior to step (a).

### C-Terminal Amine Functionalization

In another aspect, the present disclosure provides a method of selective C-terminal amine functionalization of a peptide, comprising:

a. reacting a plurality of peptides of Formula (VI):

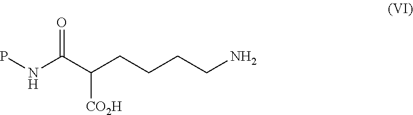

or salts thereof, with a compound of Formula (VII):

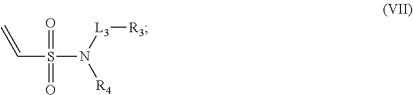

to obtain a plurality of compounds of Formula (VIII):

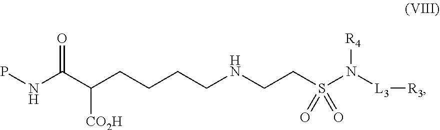

or salts thereof; and

b. reacting the plurality of compounds of Formula (VIII), or salts thereof, with a compound of Formula (IX):

R₅-L₄-Z₁; (IX)

to afford a plurality of compounds of Formula (X):

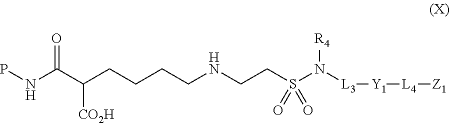

or salts thereof; wherein P, L₃, L₄, R₃, R₄, Y₁, and Z₁ are as defined below.

Each P independently is a peptide. In certain embodiments, P has 2-100 amino acid residues. In certain embodiments, P has 2-30 amino acid residues.

L₃ is a linker. In certain embodiments, L₃ is a substituted or unsubstituted aliphatic chain, wherein one or more carbon atoms are optionally, independently replaced by a heteroatom, an aryl, heteroaryl, cycloalkyl, or heterocyclyl moiety. In certain embodiments, L₃ is polyethylene glycol (PEG). In other embodiments, L₃ is a peptide, or an oligonucleotide.

L₄ is a linker, or is absent. In certain embodiments, L₄ is absent. In certain embodiments, L₄ is a substituted or unsubstituted aliphatic chain, wherein one or more carbon atoms are optionally, independently replaced by a heteroatom, an aryl, heteroaryl, cycloalkyl, or heterocyclyl moiety. In certain embodiments, L₄ is polyethylene glycol (PEG). In other embodiments, L₄ is a peptide, or an oligonucleotide.

R₃ is a moiety comprising a click chemistry handle. In certain embodiments, R₃ is a moiety comprising an azide, tetrazine, nitrile oxide, alkyne or strained alkene. In certain embodiments, the alkyne is a primary alkyne. In certain embodiments, the alkyne is a cyclic (e.g., mono- or polycyclic) alkyne (e.g., diarylcyclooctyne, or bicycle[6.1.0]nonyne). In certain embodiments, the strained alkene is trans-cyclooctene. In certain embodiments, R1 is a moiety comprising an azide. In certain embodiments, the tetrazine comprises the structure:

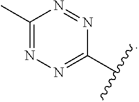 [SMILES: *c1nnc(C)nn1]

R₄ is substituted or unsubstituted aryl or substituted or unsubstituted heteroaryl. In certain embodiments, R₄ is substituted or unsubstituted phenyl. In certain particular embodiments, R₄ is phenyl. In certain particular embodiments, R₄ is 4-nitrophenyl.

R₅ is a moiety comprising a click chemistry handle that is complementary to R₃. The click chemistry handle of R₅ is capable of undergoing a click reaction (i.e., an electrocyclic reaction to form a 5-membered heterocyclic ring) with R₃. For example, when R₃ comprises an azide, nitrile oxide, or a tetrazine, then R₅ may comprise an alkyne or a strained alkene. Conversely, when R₃ comprises an alkyne or a strained alkene, then R₅ may comprise an azide, nitrile oxide, or tetrazine. In certain embodiments, R₅ is a moiety comprising an azide, tetrazine, nitrile oxide, alkyne or strained alkene. In certain embodiments, the alkyne is a primary alkyne. In certain embodiments, the alkyne is a cyclic (e.g., mono- or polycyclic) alkyne (e.g., diarylcyclooctyne, or bicycle[6.1.0]nonyne). In certain particular embodiments, R₅ comprises BCN. In other particular embodiments, R₅ comprises DBCO. In certain embodiments, the strained alkene is trans-cyclooctene. In certain embodiments, the tetrazine comprises the structure:

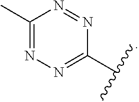 [SMILES: *c1nnc(C)nn1]

Y₁ is a moiety resulting from the click reaction of R₃ and R₅. Y₁ is a 5-membered heterocyclic ring resulting from an electrocyclic reaction (e.g., 3+2 cycloaddition, or 4+2 cycloaddition) between the reactive click chemistry handles of R₃ and R₅. In certain embodiments, Y₁ is a diradical comprising a 1,2,3-triazolyl, 4,5-dihydro-1,2,3-triazolyl, isoxazolyl, 4,5-dihydroisoxazolyl, or 1,4-dihydropyridazyl moiety.

Z₁ is a water-soluble moiety. In certain embodiments, Z₁ imparts water-solubility to the compound to which it is attached. In certain embodiments, Z₁ comprises polyethylene glycol (PEG). In certain embodiments, Z₁ comprises single-stranded DNA. In certain particular embodiments, Z₁ comprises Q24. In certain embodiments, Z₁ comprises single-stranded DNA. In certain embodiments (e.g., compounds of Formula (V)), Z₁ further comprises biotin (e.g., bisbiotin). When Z₁ comprises biotin (e.g., bisbiotin), Z₁ may further comprise streptavidin. In certain embodiments, Z₁ comprises double-stranded DNA. In some embodiments, the moieties of Z₁ are capable of intermolecularly binding another molecule or surface, e.g., to anchor a compound comprising Z₁ to the molecule or surface.

In certain embodiments, the compound of Formula (VII) is selected from:

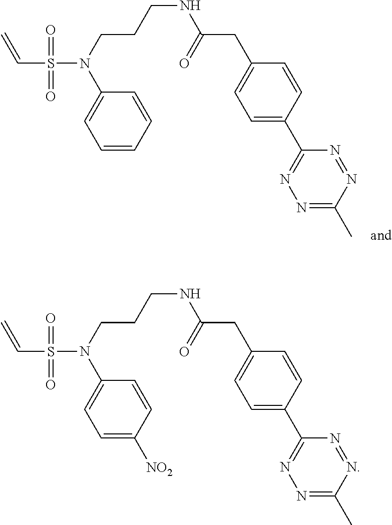

In certain embodiments, Formula (VIII) is of Formula (VIIIa) or Formula (VIIIb):

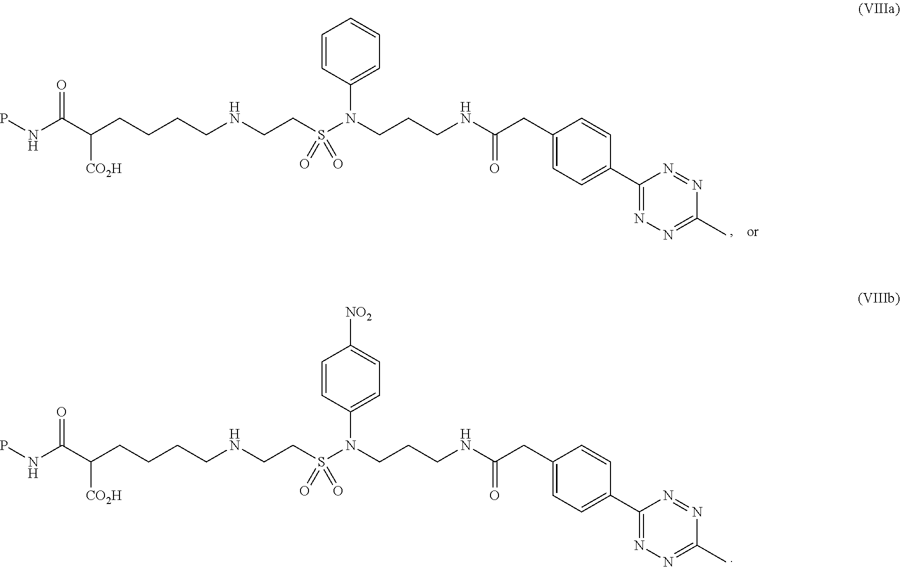

In certain embodiments, Formula (IX) comprises TCO, single-stranded DNA, and biotin (e.g., bisbiotin). In certain embodiments, Formula (IX) is Q24-BisBt-BCN. In certain embodiments, Formula (IX) is Q24-BisBt-DBCO. In certain embodiments, Formula (IX) is Q24-BisBt-TCO. Generally, Formula (IX) may comprise a branching moiety (e.g., a 1, 3, 5-tricarboxylate moiety), wherein two branches are direct or indirect attachments to biotin moieties, and the third branch is an attachment to the water soluble moiety (e.g., a polynucleotide such as Q24). As shown in FIG. 4 and FIG. 5, in certain embodiments Formula (IX) comprises a triazole moiety derived from the click-coupling of fragments comprising (i) a bisbiotin-azide functionalized linker and (ii) an alkyne (e.g., BCN)-functionalized polynucleotide (e.g. Q24). The click-coupled product may be derivatized to introduce a further click handle R₅, such as BCN or DBCO.

In certain embodiments, the reaction of step (a) is performed in the presence of a buffer having a concentration in the range of about 20 mM-500 mM and a pH in the range of about 9-11, and acetonitrile in the range of about 20-70% of total volume. In certain embodiments, the reaction of step (a) is performed in pH 9.5 buffer/acetonitrile (1:3 v/v) at approximately 37° C. In certain embodiments, the reaction of step (a) is performed using a concentration of the compound of Formula (VII) of about 500 μM-50 mM.

In certain embodiments, the plurality of compounds of Formula (VIII) is enriched prior to step (b). In certain embodiments, the enrichment comprises ethyl acetate/hexane extraction. Suitable ranges for ethyl acetate/hexane include, but are not limited to, 20 to 100 volume % ethyl acetate in hexanes. In certain embodiments, the volume of organic solvent used in the extraction is about 10× the volume of aqueous layer. Other water immiscible organic solvents can be used in the extraction, e.g., diethyl ether, dichloromethane, chloroform, benzene, toluene, and n-1-butanol.

In certain embodiments, the reaction of step (b) comprises reacting the compounds of Formula (VIII) with about one equivalent of the compound of Formula (IX). In certain embodiments, the reaction of step (b) comprises heating the reaction mixture.

In certain embodiments of step (b), when Z₁ comprises single-stranded DNA, the method further comprises hybridizing a complementary DNA strand to the single-stranded DNA to obtain a compound wherein Z₁ comprises double-stranded DNA. In certain embodiments, the single-stranded DNA is Q24 and the complementary DNA strand is a Cy3B-labeled Q24 complementary strand.

In certain embodiments of step (b), when Z₁ comprises biotin (e.g., bisbiotin), the method further comprises contacting the biotin (e.g., bisbiotin) with streptavidin to obtain a compound wherein Z₁ comprises biotin (e.g., bisbiotin) and streptavidin.

In certain embodiments, the plurality of peptides of Formula (VI), or salts thereof, is obtained by subjecting a protein to enzymatic digestion to obtain a digestive mixture comprising the plurality of peptides of Formula (VI), or salts thereof. The enzymatic digestion comprises cleaving the C-terminal bonds of lysine and/or arginine residues of the protein. In certain embodiments, the enzymatic digestion is performed using Trypsin, Lys-C, or a combination thereof. In certain embodiments, the enzymatic digestion comprises reacting the protein with Trypsin and Lys-C in Tris-HCl buffer (pH 8.5). In certain embodiments, the total concentration of the plurality of peptides of Formula (VI), or salts thereof, after digestion of 20 μg protein is below 100 μM. In certain embodiments, the enzymatic digestion comprises reacting the protein with Trypsin, wherein the molar ratio of Trypsin:protein is in the range of 1:50 to 1:200, inclusive.

In certain embodiments, the sulfide moieties of the protein are protected prior to enzymatic digestion. In certain specific embodiments, the sulfide moieties are protected by exposing the protein to tris(carboxyethyl)phosphine (TCEP) and iodoacetamide (ICM), or maleimide.

In certain embodiments, the method further comprises the step of enriching the digestive mixture prior to step (a). In certain embodiments, the digestive mixture is used in the method of selective C-terminal amine functionalization of a peptide without enrichment or purification.

### Amino Acid Side Chain Derivatization Via Diazo Transfer

Prior to sequencing, digested peptides must be functionalized with a moiety that is capable of immobilizing the peptides on the sequencing substrate. In some embodiments, is achieved by amino acid side chain derivatization. Accordingly, the present disclosure provides a method of selective N-functionalization of an amino acid side chain of a peptide, comprising reacting a plurality of peptides of Formula (XI):

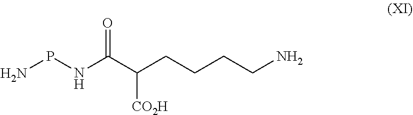

or salts thereof, wherein each P independently is a peptide having an N-terminal amine, with a derivatization agent such as a compound of Formula (XII):

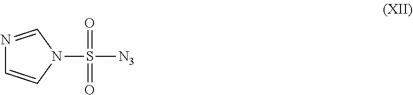 [SMILES: [N-]=[N+]=NS(=O)(=O)n1ccnc1]

under conditions (a), comprising Cu²⁺, or a precursor thereof, and a buffer having a pH of about 7-8.5; to obtain a plurality of N-terminal azido compounds of Formula (XIIIa):

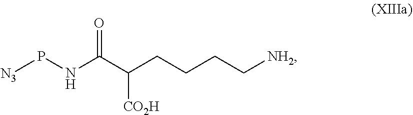

or salts thereof; or
under conditions (b), comprising Cu²⁺, or a precursor thereof, and a buffer having a pH of about 10-11; to obtain a plurality of ε-azido compounds of the Formula (XIII):

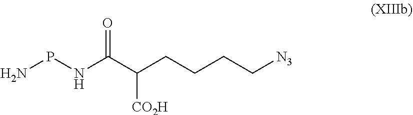

or salts thereof.

In some embodiments, the derivatization agent of Formula (XII) is present in the form of a salt. In certain particular embodiments, the compound of Formula (XII) is imidazole-1-sulfonyl azide tetrafluoroborate. In some embodiments, the compound of Formula (XII), or salt thereof, is present in the form of a reagent solution. In certain embodiments, the reagent solution comprises a pH adjusting reagent (e.g., potassium hydroxide).

In the context of the present disclosure, a pH adjusting reagent may comprise any chemical suitable for adjusting the pH of a solution to a desired value for a chemical reaction. In some embodiments, a pH adjusting reagent comprises a base (e.g. a strong base, a weak base). In some embodiments, a pH adjusting reagent comprises an acid (e.g. strong acid, a weak acid). In some embodiments, a pH adjusting reagent comprises a buffer.

Each P independently is a peptide having an N-terminal amine. In certain embodiments, P has 2-100 amino acid residues. In certain embodiments, P has 2-30 amino acid residues. In some embodiments, the concentration of a peptide in the reaction is any conceivable concentration necessary.

In certain embodiments, conditions (a) comprise a catalytic reagent such as a suitable Cu²⁺ salt, for example CuSO₄. In certain embodiments, conditions (a) comprise reaction at about 25° C. for about 30-60 minutes. In a particular embodiment, conditions (a) comprise reaction at ambient temperature (e.g., about 25° C.) for about 60 minutes.

In certain embodiments, the compound of Formula (XII) is replaced by an aryl/heteroaryl sulfonyl azide compound that is not larger than 500 Da. For example, the compound of Formula (XII) may be replaced with a compound of Formula (XIIa):

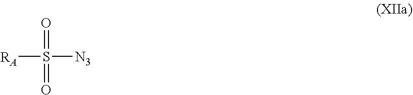

wherein R_{A} is substituted or unsubstituted aryl, or substituted or unsubstituted heteroaryl.

In certain embodiments, conditions (b) include comprise phosphate or bicarbonate buffer at pH 10.5. In certain embodiments, conditions (b) include a suitable pH adjusting reagent (e.g., potassium carbonate). In certain embodiments, conditions (b) comprise a suitable catalytic reagent such as a Cu²⁺ salt, for example CuCl₂, CuBr₂, Cu(OH)₂, or CuSO₄. In a particular embodiment, the Cu²⁺ salt is CuSO₄. In certain embodiments, the molar amount of the Cu²⁺ salt is about 2.5 times the molar amount of the compound of Formula (XI). In certain particular embodiments, conditions (b) comprise that the concentration of the Cu²⁺ salt is about 250 μM. In some embodiments, conditions (b) comprise that the concentration of the Cu²⁺ salt is between 1-5 mM or 100-1000 μM.

In certain embodiments, conditions (b) further comprise reaction at about 20-30° C., e.g., 20-25° C., 22-27° C., 25-30° C., 20° C., 21° C., 22° C., 23° C., 24° C., 25° C., 26° C., 27° C., 28° C., 29° C., or 30° C.

In certain embodiments, conditions (b) further comprise reaction for about 30-60 minutes, e.g., 30-35 minutes, 35-40 minutes, 40-45 minutes, 45-50 minutes, 50-55 minutes, or 55-60 minutes. In a particular embodiment, conditions (b) comprise reaction at ambient temperature (e.g., about 25° C.) for about 60 minutes.

In some embodiments, the compound of Formula (XIIa) is present in the form of a solution. In certain embodiments, the solution comprises a base (e.g., potassium hydroxide).

In certain embodiments, the N-terminal:ε selectivity of the diazo transfer reaction under conditions (b) is at least about 90%.

### Quenching of Diazo Transfer Reactions

In certain embodiments, methods utilizing diazo transfer chemistry as described herein further comprise the step of quenching (i.e., neutralizing) unreacted sulfonyl azide agent by addition of a material which neutralizes the sulfonyl azide agent. In certain embodiments, the material is a resin or bead, e.g., a polystyrene bead. In certain embodiments, the material comprises functional groups, e.g. a polystyrene polyamine bead. Advantageously, a resin or bead may be removed by filtration. In some embodiments, the plurality of peptides of Formula (XI), or salts thereof, is obtained by subjecting a protein to enzymatic digestion, to obtain a digestive mixture comprising the plurality of peptides of Formula (XI), or salts thereof. The enzymatic digestion comprises cleaving the C-terminal bonds of aspartic acid and/or glutamic acid residues of the protein.

In some embodiments, the enzymatic digestion is Trypsin+Lys-C digestion. In some embodiments, the Trypsin+Lys-C digestion comprises reacting the protein with Trypsin and Lys-C at room temperature in pH 9.5 buffer.

In some embodiments, the method further comprises enriching the plurality of compounds of Formula (XIIIb), or salts thereof.

### Immobilization Complex Formation

In some embodiments, the method further comprises reacting the plurality of compounds of Formula (XIIIb) or salts thereof with an immobilization complex, such as a compound of Formula (XIV):

R₆-L₅-Z₂ (XIV)

wherein R₆ is a moiety comprising an alkyne or a strained alkene; L₅ is a linker or is absent; and Z₂ is a water-soluble moiety;

to obtain a plurality of compounds of Formula (XV), or salts thereof:

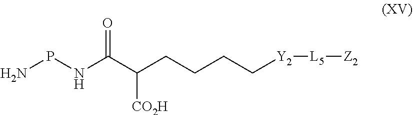

wherein Y₂ is a moiety resulting from a click reaction with the azide moiety of Formula (XIIIb) and R₆.

R₆ is a moiety comprising a click chemistry handle that is complementary to the azide moiety of Formula (XIIIb). The click chemistry handle of R₆ is capable of undergoing a click reaction (i.e., an electrocyclic reaction to form a 5-membered heterocyclic ring) with the azide moiety of Formula (XIIIb). In certain embodiments, R₆ comprises an alkyne or a strained alkene. In certain embodiments, the alkyne is a primary alkyne. In certain embodiments, the alkyne is a cyclic (e.g., mono- or polycyclic) alkyne (e.g., diarylcyclooctyne, or bicycle[6.1.0]nonyne). In certain particular embodiments, R₆ comprises BCN. In other particular embodiments, R₆ comprises DBCO. In certain embodiments, the strained alkene is trans-cyclooctene.

In certain embodiments, L₅ is absent. In certain embodiments, L₅ is a substituted or unsubstituted aliphatic chain, wherein one or more carbon atoms are optionally replaced by a heteroatom, an aryl, heteroaryl, cycloalkyl, or heterocyclyl moiety. In certain embodiments, L₅ is polyethylene glycol (PEG). In other embodiments, L₅ is a peptide, or an oligonucleotide.

In certain embodiments, Z₂ comprises PEG. In certain embodiments, Z₂ comprises single-stranded DNA. In certain embodiments, Z₂ comprises double-stranded DNA. In certain embodiments, Z₂ further comprises biotin (e.g., bisbiotin). In certain embodiments, when Z₂ comprises single-stranded DNA, the method further comprises hybridizing a complementary DNA strand to the single-stranded DNA to obtain a compound wherein Z₂ comprises double-stranded DNA. In certain embodiments, the single-stranded DNA is Q24 and the complementary DNA strand is Cy3B.

In certain embodiments, the compound of Formula (XIV) is an immobilization complex. In certain embodiments, the compound of Formula (XIV) comprises TCO, single-stranded DNA, and biotin (e.g., bisbiotin). In certain embodiments, Formula (XIV) is Q24-BisBt-BCN. In certain embodiments, Formula (XIV) is Q24-BisBt-DBCO. In certain embodiments, Formula (XIV) is Q24-BisBt-TCO. Generally, Formula (XIV) may comprise a branching moiety (e.g., a 1, 3, 5-tricarboxylate moiety), wherein two branches are direct or indirect attachments to biotin moieties, and the third branch is an attachment to the water soluble moiety (e.g., a polynucleotide such as Q24). As shown in FIG. 4 and FIG. 5, in certain embodiments Formula (XIV) comprises a triazole moiety derived from the click-coupling of fragments comprising (i) a bisbiotin-azide functionalized linker and (ii) an alkyne (e.g., BCN)-functionalized polynucleotide (e.g. Q24). The click-coupled product may be derivatized to introduce a further click handle R₆, such as BCN or DBCO.

In another embodiment, the immobilization complex of Formula (XIV) comprises DBCO, single-stranded DNA, and streptavidin (SV). In certain particular embodiments, the compound of Formula (XIV) is DBCO-Q24-SV.

In certain embodiments, when Z₂ comprises biotin (e.g., bisbiotin), the method further comprises contacting the biotin (e.g., bisbiotin) with streptavidin to obtain a compound wherein Z₂ comprises biotin (e.g., bisbiotin) and streptavidin. In other embodiments, when Z₂ comprises streptavidin, the method further comprises contacting the streptavidin with biotin (e.g., bisbiotin) to obtain a compound wherein Z₂ comprises streptavidin and biotin (e.g., bisbiotin).

### Click Chemistry

In certain embodiments, the reaction used to conjugate the host to the tag is a “click chemistry” reaction (e.g., the Huisgen alkyne-azide cycloaddition). It is to be understood that any “click chemistry” reaction known in the art can be used to this end. Click chemistry is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2] cycloaddition).

The term “click chemistry” refers to a chemical synthesis technique introduced by K. Barry Sharpless of The Scripps Research Institute, describing chemistry tailored to generate covalent bonds quickly and reliably by joining small units comprising reactive groups together. See, e.g., Kolb, Finn and Sharpless Angewandte Chemie International Edition (2001) 40: 2004-2021; Evans, Australian Journal of Chemistry (2007) 60: 384-395). Exemplary reactions include, but are not limited to, azide-alkyne Huisgen cycloaddition; and Diels-Alder reactions (e.g., tetrazine [4+2] cycloaddition). In some embodiments, click chemistry reactions are modular, wide in scope, give high chemical yields, generate inoffensive byproducts, are stereospecific, exhibit a large thermodynamic driving force >84 kJ/mol to favor a reaction with a single reaction product, and/or can be carried out under physiological conditions. In some embodiments, a click chemistry reaction exhibits high atom economy, can be carried out under simple reaction conditions, use readily available starting materials and reagents, uses no toxic solvents or use a solvent that is benign or easily removed (preferably water), and/or provides simple product isolation by non-chromatographic methods (crystallization or distillation).

The term “click chemistry handle,” as used herein, refers to a reactant, or a reactive group, that can partake in a click chemistry reaction. For example, a strained alkyne, e.g., a cyclooctyne, is a click chemistry handle, since it can partake in a strain-promoted cycloaddition (see, e.g., Table 1). In general, click chemistry reactions require at least two molecules comprising click chemistry handles that can react with each other. Such click chemistry handle pairs that are reactive with each other are sometimes referred to herein as partner click chemistry handles. For example, an azide is a partner click chemistry handle to a cyclooctyne or any other alkyne. Exemplary click chemistry handles suitable for use according to some aspects of this invention are described herein, for example, in Tables 1 and 2. Other suitable click chemistry handles are known to those of skill in the art.

TABLE 1
Exemplary click chemistry handles and reactions.

| | |
|---|---|
| 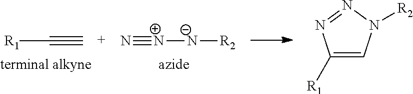 | 1,3-dipolar cycloaddition |
| 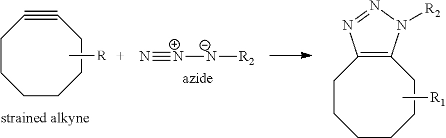 | Strain-promoted cycloaddition |
| 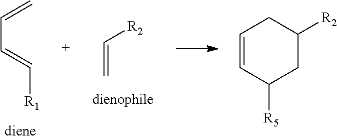 | Diels-Alder reaction |
| 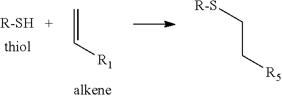 | Thiol-ene reaction |

In some embodiments, click chemistry handles are used that can react to form covalent bonds in the presence of a metal catalyst, e.g., copper (II). In some embodiments, click chemistry handles are used that can react to form covalent bonds in the absence of a metal catalyst. Such click chemistry handles are well known to those of skill in the art and include the click chemistry handles described in Becer, Hoogenboom, and Schubert, Click Chemistry beyond Metal-Catalyzed Cycloaddition, Angewandte Chemie International Edition (2009) 48: 4900-4908.

TABLE 2
Exemplary click chemistry handles and reactions. Reproduced in part from Becer,
Hoogenboom, and Schubert, Click Chemistry Beyond Metal-Catalyzed Cycloaddition,
Angewandte Chemie International Edition (2009) 48: 4900-4908.

| | Reagent A | Reagent B | Mechanism | Notes on reaction^{[a]} |
|---|---|---|---|---|
| 0 | azide | alkyne | Cu-catalyzed [3 + 2] | 2 h at 60° C. in H₂O |
| | | | azide-alkyne cycloaddition | |
| | | | (CuAAC) | |
| 1 | azide | cyclooctyne | strain-promoted [3 + 2] | 1 h at RT |
| | | | azide-alkyne cycloaddition | |
| | | | (SPAAC) | |
| 2 | azide | activated alkyne | [3 + 2] Huisgen cycloaddition | 4 h at 50° C. |
| 3 | azide | electron- | [3 + 2] cycloaddittion | 12 h at RT in H₂O |
| | | deficient alkyne | | |
| 4 | azide | aryne | [3 + 2] cycloaddition | 4 h at RT in THF with |
| | | | | crown ether or 24 h |
| | | | | at RT in CH₃CN |
| 5 | tetrazine | alkene | Diels-Alder retro-[4 + 2] | 40 min at 25° C. |
| | | | cycloaddition | (100% yield) |
| | | | | N₂ is the only by-product |
| 6 | tetrazole | alkene | 1,3-dipolar cycloaddition | few min UV irradiation |
| | | | (photoclick) | then overnight at 4° C. |
| 7 | dithioester | diene | hetero-Diels-Alder cycloaddition | 10 min at RT |
| 8 | anthracene | maleimide | [4 + 2] Diels-Alder reaction | 2 days at reflux in toluene |
| 9 | thiol | alkene | radical addition (thio click) | 30 min UV (quantitative |
| | | | | conv.) or 24 h UV |
| | | | | irradiation (>96%) |
| 10 | thiol | enone | Michael addition | 24 h at RT in CH₃CN |
| 11 | thiol | maleimide | Michael addition | 1 h at 40° C. in THF or |
| | | | | 16 h at RT in dioxane |
| 12 | thiol | para-fluoro | nucleophilic substitution | overnight at RT in DMF or |
| | | | | 60 min at 40° C. in DMF |
| 13 | amine | para-fluoro | nucleophilic substitution | 20 min MW at 95° C. in |
| | | | | NMP as solvent |

^{[a]}RT = room temperature,
DMF = N,N-dimethylformamide,
NMP = N-methylpyrolidone,
THF = tetrahydrofuran,
CH₃CN = acetonitrile.

Additional click chemistry handles suitable for use in methods of conjugation described herein are well known to those of skill in the art, and such click chemistry handles include, but are not limited to, the click chemistry reaction partners, groups, and handles described in PCT/US2012/044584 and references therein, which references are incorporated herein by reference for click chemistry handles and methodology.

### Compounds

In certain aspects, the present disclosure provides compounds of Formulae (II), (IIa), (III), (IIIa), (IV), (V), (Va), (VII), (VIII), (VIIIa), (VIIIb), (XIV), (X), (XI), (XII), (XIIIa), (XIIIb), (XV), and salts thereof, as described herein in various embodiments.

In certain embodiments, the compounds are water soluble.

### Peptide Surface Immobilization

In certain embodiments, the compounds are useful for applications relating to the analysis of proteins and peptides, such as peptide sequencing. For example, in certain embodiments, compounds of Formulae (V), (X), (XV), and salts thereof, may be covalently or non-covalently attached to a surface.

In certain analytical methods (e.g., single molecule analytical methods), a molecule to be analyzed is immobilized onto surfaces such that the molecule may be monitored without interference from other reaction components in solution. In some embodiments, surface immobilization of the molecule allows the molecule to be confined to a desired region of a surface for real-time monitoring of a reaction involving the molecule.

Accordingly, in some aspects, the application provides methods of immobilizing a peptide to a surface by attaching any one of the compounds described herein to a surface of a solid support. The solid support may be part of an article coupled to our coupleable to a detection module (e.g., sequencing module) downstream of the fluidic devices for sample preparation described herein. In some embodiments, the methods comprise contacting a compound of Formula (V), (X), (XV), or a salt thereof, to a surface of a solid support. In some embodiments, the surface is functionalized with a complementary functional moiety configured for attachment (e.g., covalent or non-covalent attachment) to a functionalized terminal end of a peptide. In some embodiments, the solid support comprises a plurality of sample wells formed at the surface of the solid support. In some embodiments, the methods comprise immobilizing a single peptide to a surface of each of a plurality of sample wells. In some embodiments, confining a single peptide per sample well is advantageous for single molecule detection methods, e.g., single molecule peptide sequencing.

As used herein, in some embodiments, a surface refers to a surface of a substrate or solid support. In some embodiments, a solid support refers to a material, layer, or other structure having a surface, such as a receiving surface, that is capable of supporting a deposited material, such as a functionalized peptide described herein. In some embodiments, a receiving surface of a substrate may optionally have one or more features, including nanoscale or microscale recessed features such as an array of sample wells. In some embodiments, an array is a planar arrangement of elements such as sensors or sample wells. An array may be one or two dimensional. A one dimensional array is an array having one column or row of elements in the first dimension and a plurality of columns or rows in the second dimension. The number of columns or rows in the first and second dimensions may or may not be the same. In some embodiments, the array may include, for example, 10², 10³, 10⁴, 10⁵, 10⁶, or 10⁷ sample wells.

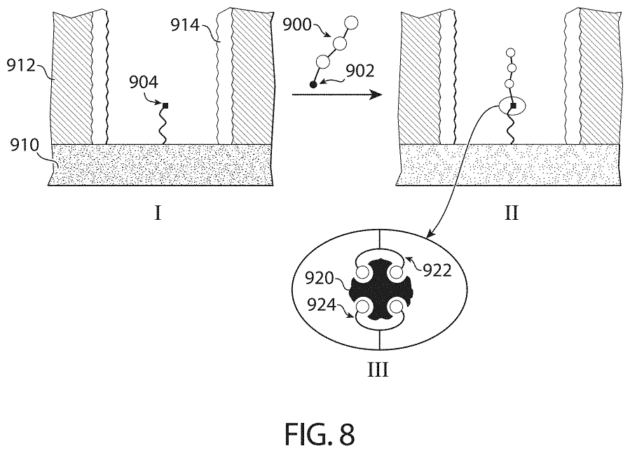

An example scheme of peptide surface immobilization is depicted in FIG. 8. As shown, panels (I)-(II) depict a process of immobilizing a peptide 900 that comprises a functionalized terminal end 902. In panel (I), a solid support comprising a sample well is shown. In some embodiments, the sample well is formed by a bottom surface comprising a non-metallic layer 910 and side wall surfaces comprising a metallic layer 912. In some embodiments, non-metallic layer 910 comprises a transparent layer (e.g., glass, silica). In some embodiments, metallic layer 912 comprises a metal oxide surface (e.g., titanium dioxide). In some embodiments, metallic layer 912 comprises a passivation coating 914 (e.g., a phosphorus-containing layer, such as an organophosphonate layer). As shown, the bottom surface comprising non-metallic layer 910 comprises a complementary functional moiety 904. Methods of selective surface modification and functionalization are described in further detail in U.S. Patent Publication No. 2018/0326412, U.S. Provisional Application No. 62/914,356, and U.S. Patent Publication No. 2021-0129179, the contents of each of which are hereby incorporated by reference.

In some embodiments, peptide 900 comprising functionalized terminal end 902 is contacted with complementary functional moiety 904 of the solid support to form a covalent or non-covalent linkage group. In some embodiments, functionalized terminal end 902 and complementary functional moiety 904 comprise partner click chemistry handles, e.g., which form a covalent linkage group between peptide 900 and the solid support. Suitable click chemistry handles are described elsewhere herein. In some embodiments, functionalized terminal end 902 and complementary functional moiety 904 comprise non-covalent binding partners, e.g., which form a non-covalent linkage group between peptide 900 and the solid support. Examples of non-covalent binding partners include complementary oligonucleotide strands (e.g., complementary nucleic acid strands, including DNA, RNA, and variants thereof), protein-protein binding partners (e.g., barnase and barstar), and protein-ligand binding partners (e.g., biotin and streptavidin).

In panel (II), peptide 900 is shown immobilized to the bottom surface through a linkage group formed by contacting functionalized terminal end 902 and complementary functional moiety 904. In this example, peptide 900 is attached through a non-covalent linkage group, which is depicted in the zoomed region of panel (III). As shown, in some embodiments, the non-covalent linkage group comprises an avidin protein 920. Avidin proteins are biotin-binding proteins, generally having a biotin binding site at each of four subunits of the avidin protein. Avidin proteins include, for example, avidin, streptavidin, traptavidin, tamavidin, bradavidin, xenavidin, and homologs and variants thereof. In some embodiments, avidin protein 920 is streptavidin. The multivalency of avidin protein 920 can allow for various linkage configurations, as each of the four binding sites are independently capable of binding a biotin molecule (shown as white circles).

As shown in panel (III), in some embodiments, the non-covalent linkage is formed by avidin protein 920 bound to a first bis-biotin moiety 922 and a second bis-biotin moiety 924. In some embodiments, functionalized terminal end 902 comprises first bis-biotin moiety 922, and complementary functional moiety 904 comprises second bis-biotin moiety 924. In some embodiments, functionalized terminal end 902 comprises avidin protein 920 prior to being contacted with complementary functional moiety 904. In some embodiments, complementary functional moiety 904 comprises avidin protein 920 prior to being contacted with functionalized terminal end 902.

In some embodiments, functionalized terminal end 902 comprises first bis-biotin moiety 922 and a water-soluble moiety, where the water-soluble moiety forms a linkage between first bis-biotin moiety 922 and an amino acid (e.g., a terminal amino acid) of peptide 900. Water-soluble moieties are described in detail elsewhere herein.

Some embodiments comprise purifying a functionalized peptide sample to form a purified functionalized peptide sample. Purifying the functionalized peptide sample may be performed in a purification region of a fifth fluidic device portion. Some embodiments comprise automatedly transporting at least some of the functionalized peptide sample from an immobilization complex forming region to the purification region. In some embodiments, purifying a functionalized peptide sample may be achieved by removing at least some of any remaining non-functionalized peptides of the functionalized peptide sample. In some embodiments, purifying comprises passing the functionalized peptide sample through a size exclusion medium. In some embodiments, the size exclusion medium may be a column. The column may be a desalting column. In some embodiments, the column is a Zeba column (e.g. a Zeba 7 kDa or a Zeba 40 kDa column). In some embodiments, the size exclusion medium is part of a fluidic device. In some embodiments, the size exclusion medium is part of a system, but is not part of a fluidic device of that system.

In some embodiments, purifying a protein comprises purification via immunoprecipitation. In some embodiments, immunoprecipitation comprises precipitating a target protein out of sample (e.g., a sample before or after functionalization) using an antibody that specifically binds to the target protein.

Certain aspects of the present disclosure are directed towards fluidic devices. The fluidic device may be a modular device that can be operably coupled with a system (e.g., a sample preparation module). In some embodiments, a fluidic device is or comprises a cartridge. Fluidic devices (and/or sample preparation modules) may contain mechanical and electronic and/or optical components which can be used to operate a fluidic device component (e.g., cartridges) as described herein. In some embodiments, the fluidic device operates to achieve and maintain specific temperatures on fluidic device portions (e.g., incubation regions). In some embodiments, the fluidic device components operate to apply specific voltages for specific time durations to electrodes of a fluidic device.

In some embodiments, a fluidic device comprises at least one channel. In some embodiments, the fluidic device comprises a microchannel. In some embodiments, at least a portion of some of the channels of the fluidic device (e.g. cartridge) have a surface comprising an elastomer configured to substantially seal off a surface opening of the channel. In some embodiments, the fluidic device components can operate to move liquids to, from, or between reservoirs and/or channels (e.g., an incubation channel) of a fluidic device. In some embodiments, the fluidic device components can operate to move liquids through channel(s) of a fluidic device, e.g., to, from, or between reservoirs and/or other channels (e.g. an incubation channel) of a fluidic device. In some embodiments, the fluidic device components move liquids via a peristaltic pumping mechanism (e.g., apparatus) that is configured to interact with an elastomeric component (e.g., surface layer comprising an elastomer) associated with a channel of a fluidic device (e.g. a cartridge) to pump fluid through the channel.

In some embodiments, the system comprises a sample preparation module, the sample preparation module comprising a peristaltic pump comprising an apparatus comprising a roller and a fluidic device (e.g. a cartridge). In some embodiments, the sample preparation module comprising a peristaltic pump comprising an apparatus comprising a roller and a crank-and-rocker mechanism connected to the roller. In some embodiments, the system comprises a sample preparation module, the sample preparation module comprising a peristaltic pump comprising a fluidic device (e.g. a cartridge) comprising a base layer having a surface comprising channels, wherein at least a portion of at least some of the channels have a substantially triangularly-shaped cross-section having a single vertex at a base of the channel and having two other vertices at the surface of the base layer. The system may comprise a detection module downstream of the sample preparation module. In some embodiments, the sample preparation region comprises more than one fluidic device. In some embodiments, the system comprises a detection module downstream from the sample preparation region of the system.

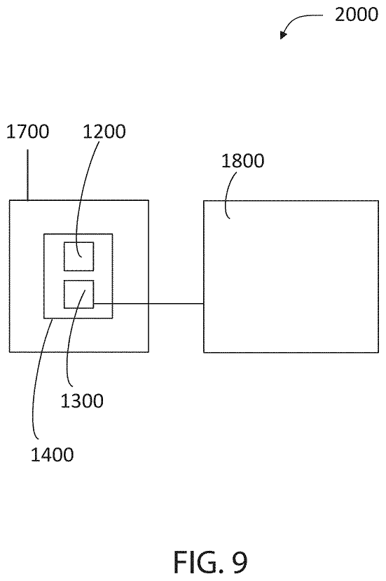

For example, FIG. 9 is a schematic illustration of an exemplary system 2000 that incorporates a device (e.g., apparatus, fluidic device, peristaltic pump) described herein, according to some embodiments. Exemplary system 2000 can be used for detecting one or more components of a sample, according to some embodiments. In some embodiments, system 2000 comprises sample preparation module 1700. In some embodiments, system 2000 comprises both sample preparation module 1700 and detection module 1800 downstream of sample preparation module 1700. Exemplary features and associated methods of sample preparation modules and detection modules are described in more detail below. Sample preparation module 1700 and detection module 1800 are configured such that at least a portion of a sample, after being prepared, can be transported (e.g., flowed) from sample preparation module 1700 to detection module 1800 (either directly or indirectly) where the sample is detected (e.g., analyzed, sequenced, identified, etc.), according to certain embodiments.

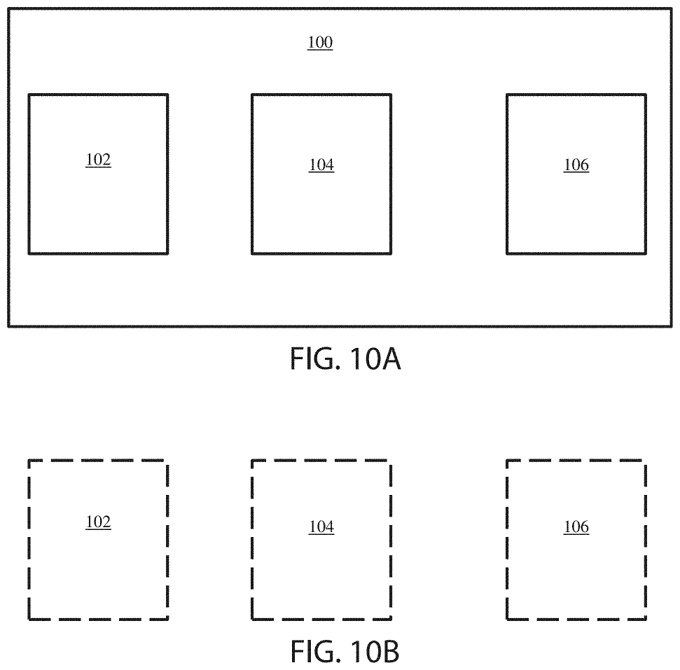

In some embodiments, two or more fluidic device portions (e.g. a first fluidic device portion, second fluidic device portion, a third fluidic device portion, a fourth fluidic device portion, a fifth fluidic device portion) described in this disclosure are part of the same fluidic device. For example: in some embodiments, the first fluidic device portion and the second fluidic device portion are part of the same fluidic device. In some embodiments, the first fluidic device portion and the third fluidic device portion are part of the same fluidic device. In some embodiments, the second fluidic device portion and the third fluidic device portion are part of the same fluidic device. In some embodiments, the first fluidic device portion, the second fluidic device portion, the third fluidic device portion, and the fourth fluidic device portion are part of the same fluidic device. For example, FIG. 10A presents a schematic illustration of first fluidic device portion 102, second fluidic device portion 104, and third fluidic device portion 106, which are part of fluidic device 100.

In some embodiments, two or more fluidic device portions (e.g. a first fluidic device portion, second fluidic device portion, a third fluidic device portion, a fourth fluidic device portion, a fifth fluidic device portion) described in this disclosure are part of separate, different fluidic devices (e.g. discrete cartridges). For example, in some embodiments the first fluidic device portion and the second fluidic device portion are part of different fluidic devices. For example, FIG. 10B presents a schematic illustration of first fluidic device portion 102, second fluidic device portion 104, and third fluidic device portion 106, which are part of separate fluidic devices. In some embodiments, fluidic device portions that are not part of the same fluidic device are part of the same system. In some embodiments, a fluidic device portion comprises one or more channels. In some embodiments, a fluidic device portion comprises one or more microchannels.

System components can include computer resources, for example, to drive a user interface where sample information can be entered, specific processes can be selected, and run results can be reported. Various aspects and embodiments of fluidic devices and systems are described in detail below.

In some embodiments, the fluidic device is or comprises a cartridge. In some embodiments, a cartridge includes one or more stored reagents (e.g., of a liquid or lyophilized form suitable for reconstitution to a liquid form). The stored reagents of a cartridge include reagents suitable for carrying out a desired process and/or reagents suitable for processing a desired sample type (e.g. a reducing agent, an amino acid side chain capping agent, a protein digestion agent). In some embodiments, a cartridge is a single-use cartridge (e.g., a disposable cartridge) or a multiple-use cartridge (e.g., a reusable cartridge). In some embodiments, a cartridge is configured to receive a user-supplied sample (e.g. of a protein). The user-supplied sample may be added to the cartridge before or after the cartridge is received by the fluidic device, e.g., manually by the user or in an automated process.

In some embodiments, a fluidic device (e.g. a cartridge) comprises a base layer having a surface comprising channels. In some embodiments, at least a portion of at least some of the channels have a substantially triangularly-shaped cross-section having a single vertex at a base of the channel and having two other vertices at the surface of the base layer. In some embodiments, at least a portion of at least some of the channels have a surface layer. The surface layer may comprise an elastomer. The surface layer may be configured to substantially seal off a surface opening of the channel. Embodiments of cartridges are further described elsewhere herein.

In some embodiments, a fluidic device (e.g. a cartridge) comprises one or more channels (e.g., microfluidic channels) configured to contain and/or transport a fluid (e.g., a fluid comprising one or more reagents) used in a sample preparation process. Reagents include buffers, enzymatic reagents, polymer matrices, capture reagents, size-specific selection reagents, sequence-specific selection reagents, and/or purification reagents. Additional reagents for use in a sample preparation process are described elsewhere herein. For example, any of the reagents (or combinations thereof) described above for sample preparation steps (e.g., for peptide or protein analysis, sequencing, or identification) may be used and/or present in the cartridge (e.g., a channel, reservoir, and/or reaction vessel of the cartridge).

In some embodiments, a fluidic device (e.g. a cartridge) includes one or more stored reagents (e.g., of a liquid or lyophilized form suitable for reconstitution to a liquid form). The stored reagents of a fluidic device (e.g. a cartridge) include reagents suitable for carrying out a desired process and/or reagents suitable for processing a desired sample type. In some embodiments, a fluidic device is a single-use fluidic device (e.g., a disposable cartridge) or a multiple-use fluidic device (e.g., a reusable cartridge). In some embodiments, a fluidic device (e.g. a cartridge) is configured to receive a user-supplied sample. The user-supplied sample may be added to the fluidic device before or after the fluidic device is received by the device, e.g., manually by the user or in an automated process.

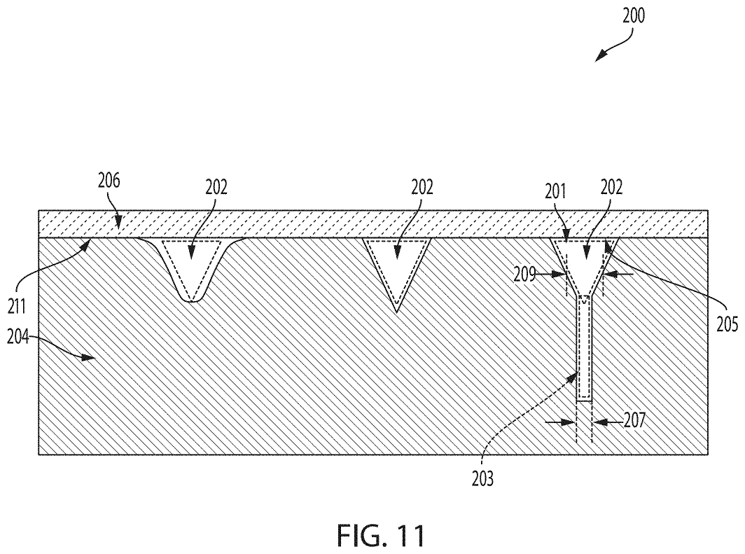

In some embodiments, a fluidic device (e.g. a cartridge) comprises a base layer. In some embodiments, a base layer has a surface comprising one or more channels. For example, FIG. 11 is a schematic diagram of a cross-section view of fluidic device 200 along the width of channels 202, in accordance with some embodiments. The depicted fluidic device 200 includes a base layer 204 having a surface 211 comprising channels 202. In certain embodiments, at least some of the channels are microchannels. For example, in some embodiments, at least some of channels 202 are microchannels. In certain embodiments, all of the channels microchannels. For example, referring again to FIG. 11, in certain embodiments, all of channels 202 are microchannels.

In some embodiments, a fluidic device is capable of handling small-volume fluids (e.g., 1-10 μL, 2-10 μL, 4-10 μL, 5-10 μL, 1-8 μL, or 1-6 μL fluid). In some embodiments, the sequencing cartridge is physically embedded or associated with a sample preparation device or module (e.g., to allow for a prepared sample to be delivered to a reaction mixture for sequencing) of a fluidic device. In some embodiments, a sequencing cartridge that is physically embedded or associated with a sample preparation device or module comprises microfluidic channels that have fluid interfaces in the form of face sealing gaskets or conical press fits (e.g., Luer fittings). In some embodiments, fluid interfaces can then be broken after delivery of the prepared sample in order to physically separate the sequencing cartridge from the sample preparation device or module.

In some embodiments, a fluidic device (e.g. a cartridge) comprises one or more reservoirs or reaction vessels configured to receive a fluid and/or contain one or more reagents used in a sample preparation process. In some embodiments, at least some channel(s) connect to a reservoir. The reservoir may be used for chemical reactions involving the sample. As one non-limiting example, the reservoir may be used for enzymatic reactions involving the sample (e.g., as an upstream process prior to further analysis, sequencing, or diagnostics processes).

The reservoir may be connected to at least some channel(s) at the bottom surface of the channel(s) by intersecting on the perimeter of the reservoir. In some such cases, then, the reservoir and the channels to which it is connected each interface with the surface layer of the fluidic device (e.g., the membrane such as a silicone membrane). However, in some embodiments, the reservoir is connected to at least some channel(s) via a top surface of the reservoir or fluidic device. In some embodiments, the reservoir is empty (e.g., initially empty prior to one or more of the processes herein). For example, the reservoir may initially be empty at the beginning of a sequencing (or analysis or diagnostic) application, but during the application, the sample and/or a reagent (e.g., an enzymatic reaction reagent) is added. In some embodiments, the reservoir contains a reagent (e.g., a small volume, such as a few microliters, of an enzymatic reaction reagent). In some such embodiments, sample is transported into the reservoir containing the reagent and the sample and the reagent mix upon transportation of the sample into the reservoir.

In some embodiments, at least some channel(s) connect to a reservoir in a temperature zone. A reservoir may be in a temperature zone if it is in contact or at least partially (or completely) surrounded by a thermal bath that can regulate the temperature of fluids in the reservoir. The incubation region described above and below may be a temperature zone. For example, the reservoir may be surround by a metal cavity (e.g., a metal cavity integrated into the instrument) capable of regulating the temperature of fluids in the reservoir. Temperature regulation of the reservoir (e.g., via a temperature zone) may allow for relatively accurate temperature control. Relatively accurate temperature may be useful in certain embodiments in which desired reactions (e.g., enzymatic reactions) proceed more efficiently at specific temperature ranges.

In some embodiments, fluidic devices comprise an incubation region. The incubation region may, in some embodiments, comprise an incubation channel. For example, referring to FIG. 1A, fluidic device 100 comprises incubation region 110, which comprises incubation channel 112. The incubation region may be configured to receive one or more reagents. For example, in some embodiments, the incubation channel is configured to receive one or more reagents. Some embodiments comprise transporting the peptide sample from a channel of the fluidic device to the incubation region prior to an incubating step. In some embodiments, the incubating step is performed while at least some of the sample is in at least a portion of an incubation channel of the incubation region.

The incubation channel may be a microchannel. In some embodiments, the incubation channel comprises a first channel portion. In some embodiments, the incubation channel comprises a second channel portion. The second channel portion may be parallel to the first channel portion. In some embodiments, a first channel portion and a second channel portion may be considered to be parallel if an angle θ between an average direction of the first channel portion and an average direction of the second channel portion is less than or equal to 20°, less than or equal to 15°, less than or equal to 10°, less than or equal to 5°, or less. In some embodiments, a first channel portion and a second channel portion are considered to be parallel are they are completely parallel (i.e. when the angle θ between an average direction of the first channel portion and an average direction of the second channel portion is zero). For example, in FIG. 1B incubation channel 112 comprises first channel portion 116, as well as second channel portion 118 which is parallel to first channel portion 116. In some embodiments, a turn portion connects the first channel portion of the second channel portion. For example, referring again to FIG. 1B, turn portion 117 connects first channel portion 116 to second channel portion 118. In some cases, at least a portion of the incubation channel has a serpentine configuration. For instance, in FIG. 1B, incubation channel 112 has a serpentine configuration. It has been realized in the context of the present disclosure that having a first channel portion and a parallel second channel portion connected via a turn portion (e.g., as in the case of a serpentine configuration) can promote efficient incubation (e.g., by efficient heating). For example, such a configuration may provide a relatively large incubation channel volume in a relatively small footprint, which can promote more efficient heating of fluid within the incubation channel. While other techniques for increasing volume are possible, the configurations described here allow for use of relatively small channel cross-sectional dimensions (e.g., microchannels) by affording relatively long path lengths within the incubation channel.

In the context of the present disclosure, a channel is considered to be serpentine if it comprises two or more parallel channel portions separated by turn portions. According to some embodiments, a serpentine channel may comprise n parallel channel portions and n−1 turn portions, configured such that a turn portion connected each consecutive pair of parallel channel portions, where n is an integer larger than one. In some embodiments, n is greater than or equal to 2, greater than or equal to 3, greater than or equal to 4, greater than or equal to 5, greater than or equal to 8, greater than or equal to 10, or greater. For example, a U-shaped channel or an S-shaped channel may be serpentine.

The incubation channel may be fluidically connected to a source of a mixture. For example, in FIG. 1D, fluidic device 100 is connected to source of mixture 114. The mixture may comprise a protein, a reducing agent, an amino acid side chain capping agent, and/or the protein digestion agent. In some embodiments the mixture may comprise all of these (e.g. the mixture may comprise a protein, a reducing agent, an amino acid side chain capping agent, and a protein digestion agent).

As used herein, the term “channel” will be known to those of ordinary skill in the art and may refer to a structure configured to contain and/or transport a fluid. A channel generally comprises: walls; a base (e.g., a base connected to the walls and/or formed from the walls); and a surface opening that may be open, covered, and/or sealed off at one or more portions of the channel. In some embodiments, a surface portion that is sealed off is completely sealed off. In some embodiments, a surface portion that is sealed off is substantially sealed off. A surface opening may be substantially sealed off if more than 50%, more than 60%, more than 75%, more than 90%, or more than 95% of the surface opening is sealed off. In some embodiments, a surface opening may be sealed off by an elastomer.

As used herein, the term “microchannel” refers to a channel that comprises at least one dimension less than or equal to 1000 microns in size. For example, a microchannel may comprise at least one dimension (e.g., a width, a height) less than or equal to 1000 microns (e.g., less than or equal to 100 microns, less than or equal to 10 microns, less than or equal to 5 microns) in size. In some embodiments, a microchannel comprises at least one dimension greater than or equal to 1 micron (e.g., greater than or equal to 2 microns, greater than or equal to 10 microns). Combinations of the above-referenced ranges are also possible (e.g., greater than or equal to 1 micron and less than or equal to 1000 microns, greater than or equal to 10 micron and less than or equal to 100 microns). Other ranges are also possible. In some embodiments, a microchannel has a hydraulic diameter of less than or equal to 1000 microns. As used herein, the term “hydraulic diameter” (DH) will be known to those of ordinary skill in the art and may be determined as: DH=4A/P, wherein A is a cross-sectional area of the flow of fluid through the channel and P is a wetted perimeter of the cross-section (a perimeter of the cross-section of the channel contacted by the fluid).

In some embodiments, at least a portion of at least some channel(s) have a substantially triangularly-shaped cross-section. In some embodiments, at least a portion of at least some channel(s) have a substantially triangularly-shaped cross-section having a single vertex at a base of the channel and having two other vertices at the surface of the base layer. Referring to FIG. 11, in some embodiments, at least a portion of at least some of channels 202 have a substantially triangularly-shaped cross-section having a single vertex at a base of the channel and having two other vertices at the surface of the base layer.

As used herein, the term “triangular” is used to refer to a shape in which a triangle can be inscribed or circumscribed to approximate or equal the actual shape, and is not constrained purely to a triangle. For example, a triangular cross-section may comprise a non-zero curvature at one or more portions.

A triangular cross-section may comprise a wedge shape. As used herein, the term “wedge shape” will be known by those of ordinary skill in the art and refers to a shape having a thick end and tapering to a thin end. In some embodiments, a wedge shape has an axis of symmetry from the thick end to the thin end. For example, a wedge shape may have a thick end (e.g., surface opening of a channel) and taper to a thin end (e.g., base of a channel), and may have an axis of symmetry from the thick end to the thin end.

Additionally, in certain embodiments, substantially triangular cross-sections (i.e., “v-groove(s)”) may have a variety of aspect ratios. As used herein, the term “aspect ratio” for a v-groove refers to a height-to-width ratio. For example, in some embodiments, v-groove(s) may have an aspect ratio of less than or equal to 2, less than or equal to 1, or less than or equal to 0.5, and/or greater than or equal to 0.1, greater than or equal to 0.2, or greater than or equal to 0.3. Combinations of the above-referenced ranges are also possible (e.g., between or equal to 0.1 and 2, between or equal to 0.2 and 1). Other ranges are also possible.

In some embodiments, at least a portion of at least some channel(s) have a cross-section comprising a substantially triangular portion and a second portion opening into the substantially triangular portion and extending below the substantially triangular portion relative to the surface of the channel. In some embodiments, the second portion has a diameter (e.g., an average diameter) significantly smaller than an average diameter of the substantially triangular portion. Referring again to FIG. 11, in some embodiments, at least a portion of at least some of channels 202 have a cross-section comprising a substantially triangular portion 201 and a second portion 203 opening into substantially triangular portion 201 and extending below substantially triangular portion 201 relative to surface 205 of the channel, wherein second portion 203 has a diameter 207 significantly smaller than an average diameter 209 of substantially triangular portion 201. In some embodiments a ratio of the diameter of the second portion to the average diameter of the substantially triangular portion is less than or equal to 0.8, less than or equal to 0.6, less than or equal to 0.5, less than or equal to 0.4, less than or equal to 0.3, less than or equal to 0.2, and/or as low as 0.1 or lower. In some such cases, the second portion of a channel having a significantly smaller diameter than that of the average diameter of the substantially triangular portion of the channel can result in the substantially triangular portion being accessible to the roller of the apparatus and deformed portions of the surface layer, but the second portion being inaccessible to the roller and deformed portions of the surface layer. For example, referring again to FIG. 11, substantially triangular portion 201 of channel 202 is accessible to a roller (not pictured) and deformed portions of surface layer 206, while second portion 203 is inaccessible to the roller and deformed portions of surface layer 206, in accordance with certain embodiments. In some such cases, a seal with the surface layer 206 cannot be achieved in portions of the channel 202 having a second portion 203, because fluid can still move freely in second portion 203, even when surface layer 206 is deformed by a roller such that it fills substantially triangular portion 201 but not second portion 203. In some embodiments, a portion along a length of a channel may have both a substantially triangular portion and a second portion (“deep section”), while a different portion along the length of the channel has only the substantially triangular portion. In some such embodiments, when the apparatus (e.g., roller) engages with the portion having both a substantially triangular portion and a second portion (deep section), pump action is not started, because a seal with the surface layer is not achieved. However, as the apparatus engages along the length direction of the channel, when the apparatus deforms the surface layer at the portion of the channel having only a substantially triangular section, pump action begins because the lack of second portion (deep section) at that portion allows for a seal (and consequently a pressure differential) to be created. Therefore, in some cases, the presence and absence of deep sections along the length of the channels of the fluidic device (e.g. cartridge) can allow for control of which portions of the channel are capable of undergoing pump action upon engagement with the apparatus.

The inclusion of such “deep sections” as second portions of at least some of the channels of the fluidic device (e.g. cartridge) may contribute to any of a variety of potential benefits. For example, such deep sections (e.g., second portion 203) may, in some cases, contribute to a reduction in pump volume in peristaltic pumping processes. In some such cases, pump volume can be reduced by a factor of two or more for higher volume resolution. In some cases, such deep sections may also provide for a well-defined starting point for the pump volume that is not determined by where the roller lands on the channel. For example, the interface between a portion of a channel having both a substantially triangular portion and a second portion (deep section) and a portion of a channel having only a substantially triangular portion can, in some cases, be used as a well-defined starting point for the pump volume, because only fluid occupying the volume of the latter channel portion can be pumped. In some cases, where the rollers lands on the channel may have some error associated depending on any of a variety of factors, such as cartridge registration. The inclusion of deep sections may, in some cases, reduce or eliminate variations in pump volume associated with such error.

As used herein, an average diameter of a substantially triangular portion of a channel may be measured as an average over the z-axis from the vertex of the substantially triangular portion to the surface of the channel.

In certain embodiments, at least some channels (also referred to herein as pumping lanes) (e.g., all channels) each comprises a valve comprising the surface layer comprising an elastomer. In certain embodiments, each valve comprises a blockage in an associated channel formed by the geometry of the end of the channel. For example, the geometry of the end of the channel may be a wall spanning from the bottom of the channel to the top surface of the channel, where the channel interfaces with the surface layer. In some such embodiments, a channel remains closed by its associated valve until enough pressure is applied such that the valve opens. In certain embodiments, the valve opens by the surface layer ballooning outward. In certain embodiments, each valve is effectively actuated by the roller. For example, in some embodiments, pressure exerted on the surface layer by the roller when the roller is relatively close to the valve causes the surface layer to balloon outward (e.g., like a diaphragm) such that a seal between the small blockage and the surface layer is reversibly broken, thereby allowing fluid to pass through the valve. In some cases, the use of such a “passive” valve can contribute to any of a variety of advantages. For example, in some instances, the use of such an integrated valve described herein can ensure that lanes that are not being pumped (e.g., via engagement with the roller of the apparatus) remain closed. In some such cases, only fluid from channels that are engaged by the apparatus (e.g., pump) is driven from the fluidic device (e.g. cartridge), which can allow for a convenient, simple, and inexpensive way to selectively drive fluids from a multi-channel pump with reduced or no contamination.

In certain embodiments, channels have certain relatively small width and depth, with an aspect ratio of depth/width of generally less than or equal to 1. In some embodiments, channel width is greater than or equal to 1 mm, greater than or equal to 1.2 mm, greater than or equal to 1.5 mm, less than or equal to 2 mm, less than or equal to 1.8 mm, and/or less than or equal to 1.6 mm. Combinations of the above-referenced ranges are also possible (e.g., between or equal to 1 mm and 2 mm). Other ranges are also possible. In some embodiments, channel depth is greater than or equal to 0.6 mm, greater than or equal to 0.75 mm, greater than or equal to 0.9 mm, less than or equal to 1.5 mm, less than or equal to 1.2 mm, and/or less than or equal to 1.0 mm. Combinations of the above-referenced ranges are also possible (e.g., between or equal to 0.6 mm and 1.5 mm). Other ranges are also possible. In some embodiments, channel aspect ratio is less than or equal to 1, less than or equal to 0.8, less than or equal to 0.6, less than or equal to 0.5, greater than or equal to 0.2, and/or greater than or equal to 0.4. Combinations of the above-referenced ranges are also possible (e.g., between or equal to 0.2 and 1). Other ranges are also possible. In certain embodiments, given tolerances and capabilities of a molding process, channels on the order of 1.5 mm wide and on the order of 0.75 mm deep may be appropriate. In certain embodiments, a channel cross-section has an aspect ratio of 1/2 with a 90 degree v-groove which provides both ease of roller access into the channel (e.g., for which a shallower v-groove may be better) and higher volume precision (e.g., for which a deeper v-groove may be better at least because the volume becomes less dependent on achieving precise planarity of the surface layer comprising the elastomer). In certain embodiments, the channel depth is on the order of the thickness of the surface layer comprising the elastomer, such that the surface layer can temporarily fill in and seal against imperfections in the channel that are likely to be some significant fraction of the channel dimensions.

In some embodiments, at least a portion of at least some channel(s) have a surface layer.

In some embodiments, a surface layer comprises an elastomer. Referring again to FIG. 11, for example, in some embodiments, at least a portion of at least some of channels 202 have a surface layer 206, comprising an elastomer, configured to substantially seal off a surface opening of channel 202. In some embodiments, at least a portion of at least some of channels 202: have a substantially triangularly-shaped cross-section having a single vertex at a base of the channel and having two other vertices at the surface of the base layer; and have a surface layer 206, comprising an elastomer, configured to substantially seal off a surface opening of channel 202.

In some embodiments, an elastomer comprises silicone. In some embodiments, the elastomer comprises silicone and/or a thermoplastic elastomer, and/or consists essentially of an elastomer.

In some embodiments, a surface layer is configured to substantially seal off a surface opening of a channel. In some embodiments, a surface layer is configured to completely seal off a surface opening of a channel such that fluid (e.g., liquid) cannot leave the channel except via an entrance or exit of the channel. In some embodiments, a surface layer is bound to a portion of a surface of a base layer (e.g., by an adhesive, by heat lamination, or any other suitable binding means). In some embodiments, a surface layer is bound to a portion of a surface of a base layer by an adhesive. In some embodiments, a surface layer is bound to a portion of a surface of a base layer by heat lamination.

As used herein, the term “seal off” refers to contact at or near the edges of an opening such that the opening is sealed.

As used herein, the term “surface opening” refers to the portion of the channel that would open the channel to a surrounding atmosphere if not covered by a surface layer. For example, a microchannel may have a surface opening.

As used herein, a surface layer may be bound to a portion of the surface of the base layer by any suitable binding means. For example, in some embodiments, a surface layer is bound to a portion of the surface of the base layer covalently, ionically, by Van der Waals interactions, by dipole-dipole interactions, by hydrogen bonding, by pi-pi stacking interactions, or by another suitable bonding means.

In some embodiments, a surface layer is held in tension directly in contact with a portion of a surface of a base layer.

As used herein, a surface (e.g., a ceiling) of a channel may correspond to an inner surface of a surface layer.

In some embodiments, at least a portion of the surface layer is flat in the absence of at least one magnitude of applied pressure. In some embodiments, an entirety of the surface layer is flat in the absence of at least one magnitude of applied pressure. For example, in some embodiments, at least a portion (or an entirety) of the surface layer is flat in the absence of engagement by the roller of the apparatus (which can cause deformation of the surface layer via the application of a pressure).

In some embodiments, at least a portion of at least some channel(s) have walls and a base comprising a material (e.g., a substantially rigid material) that is compatible with biological material. In some embodiments, at least a portion of at least some channel(s) have walls and a base comprising a substantially rigid material. For example, referring again to FIG. 11, in some embodiments, at least a portion of at least some of channels 202 have walls and a base comprising a substantially rigid material. In certain embodiments, a base comprises a material that is the same as the material of base layer 204. In certain embodiments, a base comprises a material that is different than the material of base layer 204. For example, a base may comprise a material that is different than the material of base layer 204 in instances where the walls and base of the channel are coated with the rigid material. In some embodiments, the substantially rigid material is compatible with biological material. In some embodiments, the base layer is an injection-molded part.

In some embodiments, the fluidic device comprises a derivatization region. The derivatization region may have any suitable geometry. In some embodiments, the derivatization region comprises a container. For example, the derivatization region may comprise a cylinder, a prism, a parallelepiped, a channel, or any other container of suitable volume. In some embodiments, a derivatization region may be configured to be heated or cooled. In some embodiments, it is advantageous for the derivatization region to comprise a channel (e.g. a serpentine channel), since this configuration can promote efficient thermal contact (e.g., by efficient heating). For example, such a configuration may provide a relatively large channel volume in a relatively small footprint, which can promote more efficient heating of fluid within the channel. While other techniques for increasing volume are possible, the configurations described here allow for use of relatively small channel cross-sectional dimensions (e.g., microchannels) by affording relatively long path lengths within the derivatization region.

In some embodiments, derivatization region has a volume of greater than or equal to 10 μL, greater than or equal to 100 μL, greater than or equal to 1 mL, or greater. In some embodiments, derivatization region has a volume of less than or equal to 5 mL, of less than or equal to 1 mL, of less than or equal to 100 μL, of less than or equal to 50 μL, or less. Combinations of these ranges are possible. For instance, a derivatization region may have a volume of greater than or equal to 100 μL of less than or equal to 5 mL. Fluidic devices with volumes outside these ranges are also contemplated.

In some embodiments, the derivatization region is fluidically connected to an incubation region such that fluid can be automatedly transported from the incubation region to the derivatization region without passing through the derivatization agent reservoir. In some cases, the derivatization region is configured so that fluid from the derivatization agent reservoir can the first exposed to fluid from the incubation region in the derivatization region (e.g. via mixing).

In some embodiments, the fluidic device comprises a quenching region. The quenching region may have any suitable geometry. In some embodiments, the quenching region comprises a container. For example, the quenching region may comprise a cylinder, a prism, a parallelepiped, a channel, or any other container of suitable volume. In some embodiments, a quenching region may be configured to be heated or cooled. In some embodiments, it is advantageous for the quenching region to comprise a mixing channel (e.g. a serpentine channel), since this configuration can be conducive to pump-driven mixing. For example, such a configuration may facilitate agitation of the mixture, or may simplify recirculation of the unquenched mixture from an outlet of the quenching region to an inlet of the quenching region. While other techniques for increasing volume are possible, the configurations described here allow for use of relatively small channel cross-sectional dimensions (e.g., microchannels) by affording relatively long path lengths within the quenching region.

In some embodiments, quenching region has a volume of greater than or equal to 10 μL, greater than or equal to 100 μL, greater than or equal to 1 mL, or greater. In some embodiments, quenching region has a volume of less than or equal to 5 mL, of less than or equal to 1 mL, of less than or equal to 100 μL, of less than or equal to 50 μL, or less. Combinations of these ranges are possible. For instance, a quenching region may have a volume of greater than or equal to 100 μL of less than or equal to 5 mL. Fluidic devices with volumes outside these ranges are also contemplated.

In some embodiments, the quenching region is fluidically connected to a derivatization region. In some embodiments, the quenching region is fluidically connected to a derivatization agent reservoir and/or a derivatization reagent reservoir.

Some embodiments comprise quenching an unquenched mixture in a quenching region that may comprise a solid substrate (e.g., in a quenching region of the fluidic device). In some embodiments, the solid substrate comprises a bead. In some embodiments, the solid substrate (e.g., bead) is packed into the quenching region. In some embodiments, the solid substrate is associated with (e.g., attached to, embedded in, adjacent to, etc.) a filter within the quenching region. The solid substrate may comprise functional groups. For example, in some embodiments, the solid substrate comprises a plurality of beads, some or all of which have surfaces comprising such functional groups. In some embodiments, the functional groups of the solid substrate comprise amine groups. In some embodiments, the solid substrate comprises a polyamine bead. In some embodiments, the solid substrate comprises a plurality of polyamine beads. In some embodiments, the solid substrate is or comprises a polymeric material (e.g., a bead comprising a polymeric material). In some embodiments, the solid substrate comprises polystyrene. For example, the solid substrate may comprise a plurality of polystyrene beads (e.g., comprising functional groups such as amine groups). In some embodiments, quenching comprises reacting at least some of an excess derivatization agent of the unquenched mixture. The excess derivatization agent may be reacted with functional groups of the solid substrate (e.g., amine groups of beads within the quenching region).

In some embodiments, a fluidic device is configured to recirculate the unquenched mixture through at least a portion of the quenching region. Recirculating an unquenched mixture through at least a portion of the quenching region may offer several advantages. For instance, recirculating an unquenched mixture through at least a portion of the quenching region can increase the rate of quenching, in some embodiments. In some embodiments, the unquenched region comprises an inlet and an outlet. For example, the exemplary embodiment in FIG. 2B comprises inlet 136 and outlet 138. In some cases, the fluidic device is configured such that a fluid can be transported from the outlet quenching region to the inlet of the quenching region. For example, in FIG. 2B, fluidic device 100 is configured such that a fluid can be transported from outlet 138 of quenching region 130 to inlet 136 of quenching region 130. In some embodiments, the fluid transported from the outlet of the quenching region to the inlet of the quenching region comprises an unquenched mixture.

In some embodiments, quenching comprises keeping the unquenched mixture stationary in the presence of the solid substrate (e.g. a plurality of beads). A person of ordinary skill in the art would understand that in this context the fact that a mixture is stationary means that net flow rate of the mixture is zero. Stationary mixtures may still experience convective or turbulent flows that are not associated with that flow of the mixture. In some embodiments, quenching comprises actively mixing the unquenched mixture with the solid substrate, e.g. by producing a nonzero flow of the mixture. In some embodiments, quenching comprises multiple steps, wearing during some steps the unquenched mixture is stationary in the presence of the solid substrate, and during other steps the unquenched mixture is actively mixed with the solid substrate. For example, in some embodiments quenching comprises more than 1 step, more than 2 steps, more than 3 steps, more than 4 steps, more than 5 steps, more than 7 steps, more than 10 steps, more than 15 steps, more than 20 steps, or more steps. In some embodiments, quenching comprises alternating steps of stationary mixing and active mixing.

In some embodiments, a fluidic device further comprises a seal plate. In some embodiments, a seal plate comprises a hard plastic, and/or is an injection-molded part. In certain embodiments, a seal plate comprises one or more through-holes. In some embodiments, the one or more through-holes have a shape substantially similar to one or more associated channels in the base layer. It should be understood that in this context, the “through-holes” refer to gaps/holes/voids in the seal plate through which one or more mechanical components of, for example, an apparatus, can travel to engage and/or disengage with a surface layer of the fluidic device. For example, a peristaltic pump comprising a roller and a fluidic device (e.g. a cartridge) as described herein may be configured such that the roller travels through at least a portion of the through holes of the seal plate to reach a surface layer of the fluidic device when engaging and/or disengaging with that surface. The through-holes may have any of a variety of shapes and aspect ratios (rectangular, square, circular, oblong, etc.).

In certain embodiments, at least some of the one or more through-holes of the seal plate are configured in alignment with one or more associated channels in the base layer. In some embodiments, the fluidic device (e.g., cartridge) comprises a surface layer comprising an elastomer disposed between the seal plate and the base layer. In certain embodiments, the surface layer is disposed directly between the seal plate in the base layer. In certain embodiments, a fluidic device (e.g., cartridge) comprises one or more exposed regions of a surface layer disposed between the seal plate and a base layer, wherein each of the one or more exposed regions are defined by an associated through-hole of the seal plate and an aligned channel of the base layer. In certain embodiments, one or more exposed portions of the one or more exposed regions of the surface layer can be deformed by a roller to contact one or more associated portions of the walls and/or base of the associated channel of the base layer.

In some embodiments, a system herein comprising a sample preparation module further comprises a sequencing module. In some embodiments, a system that comprises a sample preparation module and a sequencing module involves a sequencing chip or cartridge that is embedded into a sample preparation cartridge, such that the two cartridges comprise a single, inseparable consumable. In some embodiments, the sequencing chip or cartridge requires consumable support electronics (e.g., a PCB substrate with wirebonds, electrical contacts). The consumable support electronics may be in direct physical contact with the sequencing chip or cartridge. In some embodiments, the sequencing chip or cartridge requires an interface for a peristaltic pump, temperature control and/or electrophoresis contacts. These interfaces may allow for precise geometric registration for the many electrical contacts and laser alignment. In some embodiments, different sections of a chip or cartridge may comprise different temperatures, physical forces, electrical interfaces of varying voltage and current, vibration, and/or competing alignment requirements. In some embodiments, disparate instrument sub-systems associated with either the sample preparation or sequencing module must be in close proximity in order to share resources. In some embodiments, a system that comprises a sample preparation module and a sequencing module is hands-free (i.e., can be used without the use of hands).

In some embodiments, a sample preparation device or module is used to prepare a sample for diagnostic purposes. In some embodiments, a sample preparation device that is used to prepare a sample for diagnostic purposes is positioned to deliver or transfer to a diagnostic module or diagnostic device a target molecule or a plurality of molecules (e.g., target proteins). In some embodiments, a sample preparation device or module is connected directly to (e.g., physically attached to) or indirectly to a diagnostic device.

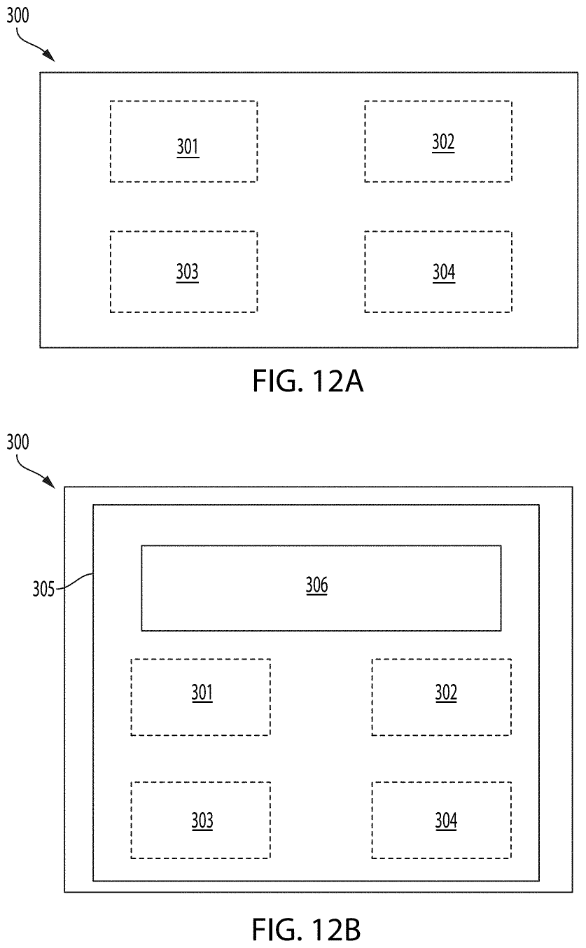

In some embodiments, a system comprises a fluidic device housing that is configured to receive one or more fluidic devices (e.g., configured to receive one cartridge at a time). FIG. 12A shows a schematic diagram of sample preparation device 300, in accordance with some embodiments. A device (e.g., a sample preparation device comprising a cartridge housing) may be configured to receive one or more cartridges (or two or more, or three or more, and so on) either sequentially or simultaneously. Sample preparation device 300, for example, can be configured to receive one or more of lysis cartridge 301, enrichment cartridge 302, fragmentation cartridge 303, and/or functionalization cartridge 304 simultaneously or sequentially.

Samples and reagents may be made to flow (e.g., through channels) in the fluidic device (e.g. cartridge) via any of a variety of techniques. One such technique is causing flow via peristaltic pumping. In some embodiments, the sample preparation module comprises a pump. In some embodiments, the pump is peristaltic pump. Some such pumps comprise one or more of the inventive components for fluid handling described herein. For example, the pump may comprise an apparatus and/or a fluidic device (e.g., cartridge). In some embodiments, the apparatus of the pump comprises a roller, a crank, and a rocker. In some such embodiments, the crank and the rocker are configured as a crank-and-rocker mechanism that is connected to the roller. The coupling of a crank-and-rocker mechanism with the roller of an apparatus can, in some cases, allow for certain of the advantages describe herein to be achieved (e.g., facile disengagement of the apparatus from the fluidic device, well-metered stroke volumes). In certain embodiments, the fluidic device of the pump comprises channels (e.g., microfluidic channels). In some embodiments, at least a portion of the channels of the fluidic device have certain cross-sectional shapes and/or surface layers that may contribute to any of a number of advantages described herein.

One non-limiting aspect of some fluidic devices (e.g. cartridges) that may, in some cases, provide certain benefits is the inclusion of channels having certain cross-sectional shapes in the fluidic devices. For example, in some embodiments, the fluidic device comprises v-shaped channels. One potentially convenient but non-limiting way to form such v-shaped channels is by molding or machining v-shaped grooves into the fluidic device. The recognized advantages of including a v-shaped channel (also referred to herein as a v-groove or a channel having a substantially triangularly-shaped cross-section) in certain embodiments in which a roller of the apparatus engages with the fluidic device to cause fluid flow through the channels. For example, in some instances, a v-shaped channel is dimensionally insensitive to the roller. In other words, in some instances, there is no single dimension to which the roller (e.g., a wedge shaped roller) of the apparatus must adhere in order to suitably engage with the v-shaped channel. In contrast, certain conventional cross sectional shapes of the channels, such as semicircular, may require that the roller have a certain dimension (e.g., radius) in order to suitably engage with the channel (e.g., to create a fluidic seal to cause a pressure differential in a peristaltic pumping process). In some embodiments, the inclusion of channels that are dimensionally insensitive to rollers can result in simpler and less expensive fabrication of hardware components and increased configurability/flexibility.

The sample preparation device may further comprise a pump configured to transport components (e.g., reagents, samples) in the received fluidic devices (e.g., within a channels/reservoirs of a fluidic device or into and/or out of a fluidic device). For example, referring to FIG. 12B, sample preparation device 300 may comprise pump 305 configured to transport components in one or more of lysis cartridge 301, enrichment cartridge 302, fragmentation cartridge 303, and/or functionalization cartridge 304. In some embodiments, a pump comprises an apparatus and a received cartridge, and an interaction between the apparatus of the pump and cartridge causes fluid flow. For example, pump 305 may be a peristaltic pump, and apparatus 306 may operatively couple to a cartridge (e.g., cartridge 301) to cause fluid motion in the cartridge (e.g., when apparatus 306 comprises a roller and cartridge 301 comprises a flexible surface (e.g., elastomer surface) deformable by the roller).

In certain aspects, fluidic devices (e.g. cartridges) comprise a surface layer (e.g., a flat surface layer). One exemplary aspect relates to potentially advantageous embodiments involving layering a membrane (also referred to herein as a surface layer) comprising (e.g., consisting essentially of) an elastomer (e.g., silicone) above the v-groove, to produce, in effect, half of a flexible tube. Then, in some embodiments, by deforming the surface layer comprising an elastomer into the channel to form a pinch and by then translating the pinch, negative pressure can be generated on the trailing edge of the pinch which creates suction and positive pressure can be generated on the leading edge of the pinch, pumping fluid in the direction of the leading edge of the pinch. In certain embodiments, this pumping by interfacing a fluidic device such as a cartridge (comprising channels having a surface layer) with an apparatus comprising a roller, which apparatus is configured to carry out a motion of the roller that includes engaging the roller with a portion of the surface layer to pinch the portion of the surface layer with the walls and/or base of the associated channel, translating the roller along the walls and/or base of the associated channel in a rolling motion to translate the pinch of the surface layer against the walls and/or base, and/or disengaging the roller with a second portion of the surface layer. In certain embodiments, a crank-and-rocker mechanism is incorporated into the apparatus to carry out this motion of the roller.

A conventional peristaltic pump generally involves tubing having been inserted into an apparatus comprising rollers on a rotating carriage, such that the tubing is always engaged with the remainder of the apparatus as the pump functions. By contrast, in certain embodiments, channels in fluidic devices (e.g. cartridges) herein are linear or comprise at least one linear portion, such that the roller engages with a horizontal surface. In certain embodiments, the roller is connected to a small roller arm that is spring-loaded so that the roller can track the horizontal surface while continuously pinching a portion of the surface layer. Spring loading the apparatus (e.g., a roller arm of the apparatus) can in some cases help regulate the force applied by the apparatus (e.g., roller) to the surface layer and a channel of a fluidic device (e.g. cartridge).

In certain embodiments, each rotation of the crank in a crank-and-rocker mechanism connected to the roller provides a discrete pumping volume. In certain embodiments, it is straightforward to park the apparatus in a disengaged position, where the roller is disengaged from any fluidic device (e.g. cartridge). In certain embodiments, forward and backward pumping motions are fairly symmetrical as provided by apparatuses described herein, such that a similar amount of force (torque) (e.g., within 10%) is required for forward and backward pumping motions.

In certain embodiments, it may be advantageous to, for a particular size of apparatus, have a relatively high crank radius (e.g., greater than or equal to 2 mm, optionally including associated linkages). Consequently, it may, in certain embodiments, also be advantageous to have a relatively high stroke length (e.g., greater than or equal to 10 mm) to engage with an associated fluidic device (e.g. cartridge). Having relatively high crank radius and stroke length, in certain embodiments, ensures no mechanical interference between the apparatus and the fluidic device when moving components of the apparatus relative to the fluidic device.

In certain embodiments, having v-shaped grooves advantageously allows for utilization with rollers of a variety of sizes having a wedge-shaped edge. By contrast, for example, having a rectangular channel rather than a v-groove results in the width of the roller associated with the rectangular channel needing to be more controlled and precise in relation to the width of the rectangular channel, and results in the forces being applied to the rectangular channel needing to be more precise. Similarly, the channel(s) having a semicircular cross-section may also require more controlled and precise dimension for the width of the associated roller.

In certain embodiments, an apparatus described herein may comprise a multi-axis system (e.g., robot) configured so as to move at least a portion of the apparatus in a plurality of dimensions (e.g., two dimensions, three dimensions). For example, the multi-axis system may be configured so as to move at least a portion of the apparatus to any pumping lane location among associated fluidic device(s). For example, in certain embodiments, a carriage herein may be functionally connected to a multi-axis system. In certain embodiments, a roller may be indirectly functionally connected to a multi-axis system. In certain embodiments, an apparatus portion, comprising a crank-and-rocker mechanism connected to a roller, may be functionally connected to a multi-axis system. In certain embodiments, each pumping lane may be addressed by location and accessed by an apparatus described herein using a multi-axis system.

In certain embodiments, a system described herein for sample preparation may be fluidically connected with a diagnostic instrument for analyzing at least some of (e.g., all of) the samples prepared by the system. In some embodiments, a peptide sample (e.g. a purified peptide sample) may be automatedly transported from the sample preparation module to the diagnostic instrument. In certain embodiments, the diagnostic instrument generates an output based on the presence or absence of a band or color based on the underlying sequence of a sample. It should be understood that when components (e.g., modules, devices) are described as being connected (e.g., functionally connected), the connections may be permanently connected, or the connections may be reversibly connected. In some instances, components being described as being connected are decoupleably connected, in that they may be connected (e.g., with a fluidic connection via, for example, a channel, tube, conduit) during a first period of time, but then during a second period of time, they may not be connected (e.g., by decoupling the fluidic connection). In some such embodiments, reversible/decoupleable connections may provide for modular systems in which certain components can be replaced or reconfigured, depending on the type of sample preparation/analysis/sequencing/identification being performed.

Aspects of the instant disclosure also involve methods of protein sequencing and identification, methods of protein sequencing and identification, methods of amino acid identification, and compositions, systems, and devices for performing such methods. In some aspects, methods of determining the sequence of a target protein are described. In some embodiments, the target protein is enriched (e.g., enriched using electrophoretic methods, e.g., affinity SCODA) prior to determining the sequence of the target protein. In some aspects, methods of determining the sequences of a plurality of proteins (e.g., at least 2, 3, 4, 5, 10, 15, 20, 30, 50, or more) present in a sample (e.g., a purified sample, a cell lysate, a single-cell, a population of cells, or a tissue) are described. In some embodiments, a sample is prepared as described herein (e.g., digested, lysed, purified, fragmented, and/or enriched for a target protein) prior to determining the sequence of a target protein or a plurality of proteins present in a sample. In some embodiments, a target protein is an enriched target protein (e.g., enriched using electrophoretic methods, e.g., affinity SCODA).

In some embodiments, the instant disclosure provides methods of sequencing and/or identifying an individual protein in a sample comprising a plurality of proteins by identifying one or more types of amino acids of a protein from the mixture. In some embodiments, one or more amino acids (e.g., terminal amino acids) of the protein are labeled (e.g., directly or indirectly, for example using a binding agent) and the relative positions of the labeled amino acids in the protein are determined. In some embodiments, the relative positions of amino acids in a protein are determined using a series of amino acid labeling and cleavage steps. In some embodiments, the relative position of labeled amino acids in a protein can be determined without removing amino acids from the protein but by translocating a labeled protein through a pore (e.g., a protein channel) and detecting a signal (e.g., a Førster resonance energy transfer (FRET) signal) from the labeled amino acid(s) during translocation through the pore in order to determine the relative position of the labeled amino acids in the protein molecule.

In some embodiments, the identity of a terminal amino acid (e.g., an N-terminal or a C-terminal amino acid) is determined prior to the terminal amino acid being removed and the identity of the next amino acid at the terminal end being assessed; this process may be repeated until a plurality of successive amino acids in the protein are assessed. In some embodiments, assessing the identity of an amino acid comprises determining the type of amino acid that is present. In some embodiments, determining the type of amino acid comprises determining the actual amino acid identity (e.g., determining which of the naturally-occurring 20 amino acids an amino acid is, e.g., using a binding agent that is specific for an individual terminal amino acid). However, in some embodiments, assessing the identity of a terminal amino acid type can comprise determining a subset of potential amino acids that can be present at the terminus of the protein. In some embodiments, this can be accomplished by determining that an amino acid is not one or more specific amino acids (i.e., and therefore could be any of the other amino acids). In some embodiments, this can be accomplished by determining which of a specified subset of amino acids (e.g., based on size, charge, hydrophobicity, binding properties) could be at the terminus of the protein (e.g., using a binding agent that binds to a specified subset of two or more terminal amino acids).

In some embodiments, a protein can be digested into a plurality of smaller proteins and sequence information can be obtained from one or more of these smaller proteins (e.g., using a method that involves sequentially assessing a terminal amino acid of a protein and removing that amino acid to expose the next amino acid at the terminus) as described above.

In some embodiments, a protein is sequenced from its amino (N) terminus. In some embodiments, a protein is sequenced from its carboxy (C) terminus. In some embodiments, a first terminus (e.g., N or C terminus) of a protein is immobilized and the other terminus (e.g., the C or N terminus) is sequenced as described herein.

As used herein, sequencing a protein refers to determining sequence information for a protein. In some embodiments, this can involve determining the identity of each sequential amino acid for a portion (or all) of the protein. In some embodiments, this can involve determining the identity of a fragment (e.g., a fragment of a target protein or a fragment of a sample comprising a plurality of proteins). In some embodiments, this can involve assessing the identity of a subset of amino acids within the protein and determining the relative position of one or more amino acid types without determining the identity of each amino acid in the protein). In some embodiments amino acid content information can be obtained from a protein without directly determining the relative position of different types of amino acids in the protein. The amino acid content alone may be used to infer the identity of the protein that is present (e.g., by comparing the amino acid content to a database of protein information and determining which protein(s) have the same amino acid content).

In some embodiments, sequence information for a plurality of protein fragments obtained from a target protein or sample comprising a plurality of proteins (e.g., via enzymatic and/or chemical cleavage) can be analyzed to reconstruct or infer the sequence of the target protein or plurality of proteins present in the sample. Accordingly, in some embodiments, the one or more types of amino acids are identified by detecting luminescence of one or more labeled affinity reagents that selectively bind the one or more types of amino acids. In some embodiments, the one or more types of amino acids are identified by detecting luminescence of a labeled protein.

In some embodiments, the instant disclosure provides compositions, devices, and methods for sequencing a protein by identifying a series of amino acids that are present at a terminus of a protein over time (e.g., by iterative detection and cleavage of amino acids at the terminus). In yet other embodiments, the instant disclosure provides compositions, devices, and methods for sequencing a protein by identifying labeled amino content of the protein and comparing to a reference sequence database.

In some embodiments, the instant disclosure provides compositions, devices, and methods for sequencing a protein by sequencing a plurality of fragments of the protein. In some embodiments, sequencing a protein comprises combining sequence information for a plurality of protein fragments to identify and/or determine a sequence for the protein. In some embodiments, combining sequence information may be performed by computer hardware and software. The methods described herein may allow for a set of related proteins, such as an entire proteome of an organism, to be sequenced. In some embodiments, a plurality of single molecule sequencing reactions are performed in parallel (e.g., on a single chip or cartridge) according to aspects of the instant disclosure. For example, in some embodiments, a plurality of single molecule sequencing reactions are each performed in separate sample wells on a single chip or cartridge.

In some embodiments, methods provided herein may be used for the sequencing and identification of an individual protein in a sample comprising a plurality of proteins. In some embodiments, the instant disclosure provides methods of uniquely identifying an individual protein in a sample comprising a plurality of proteins. In some embodiments, an individual protein is detected in a mixed sample by determining a partial amino acid sequence of the protein. In some embodiments, the partial amino acid sequence of the protein is within a contiguous stretch of approximately 5-50, 10-50, 25-50, 25-100, or 50-100 amino acids.

Without wishing to be bound by any particular theory, it is expected that most human proteins can be identified using incomplete sequence information with reference to proteomic databases. For example, simple modeling of the human proteome has shown that approximately 98% of proteins can be uniquely identified by detecting just four types of amino acids within a stretch of 6 to 40 amino acids (see, e.g., Swaminathan, et al. PLoS Comput Biol. 2015, 11(2):e1004080; and Yao, et al. Phys. Biol. 2015, 12(5):055003). Therefore, a sample comprising a plurality of proteins can be fragmented (e.g., chemically degraded, enzymatically degraded) into short protein fragments of approximately 6 to 40 amino acids, and sequencing of this protein-based library would reveal the identity and abundance of each of the proteins present in the original sample. Compositions and methods for selective amino acid labeling and identifying proteins by determining partial sequence information are described in in detail in U.S. patent application Ser. No. 15/510,962, filed Sep. 15, 2015, entitled “SINGLE MOLECULE PEPTIDE SEQUENCING,” which is incorporated herein by reference in its entirety.

Sequencing in accordance with the instant disclosure, in some aspects, may involve immobilizing a protein (e.g., a target protein) on a surface of a substrate (e.g., of a solid support, for example a chip or cartridge, for example in a sequencing device or module as described herein). In some embodiments, a protein may be immobilized on a surface of a sample well (e.g., on a bottom surface of a sample well) on a substrate. In some embodiments, the N-terminal amino acid of the protein is immobilized (e.g., attached to the surface). In some embodiments, the C-terminal amino acid of the protein is immobilized (e.g., attached to the surface). In some embodiments, one or more non-terminal amino acids are immobilized (e.g., attached to the surface). The immobilized amino acid(s) can be attached using any suitable covalent or non-covalent linkage, for example as described in this disclosure. In some embodiments, a plurality of proteins are attached to a plurality of sample wells (e.g., with one protein attached to a surface, for example a bottom surface, of each sample well), for example in an array of sample wells on a substrate.

In some embodiments, the identity of a terminal amino acid (e.g., an N-terminal or a C-terminal amino acid) is determined, then the terminal amino acid is removed, and the identity of the next amino acid at the terminal end is determined. This process may be repeated until a plurality of successive amino acids in the protein are determined. In some embodiments, determining the identity of an amino acid comprises determining the type of amino acid that is present. In some embodiments, determining the type of amino acid comprises determining the actual amino acid identity, for example by determining which of the naturally-occurring 20 amino acids is the terminal amino acid is (e.g., using a binding agent that is specific for an individual terminal amino acid). In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine. In some embodiments, determining the identity of a terminal amino acid type can comprise determining a subset of potential amino acids that can be present at the terminus of the protein. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be at the terminus of the protein (e.g., using a binding agent that binds to a specified subset of two or more terminal amino acids).

In some embodiments, assessing the identity of a terminal amino acid type comprises determining that an amino acid comprises a post-translational modification. Non-limiting examples of post-translational modifications include acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination.

In some embodiments, a protein or protein can be digested into a plurality of smaller proteins and sequence information can be obtained from one or more of these smaller proteins (e.g., using a method that involves sequentially assessing a terminal amino acid of a protein and removing that amino acid to expose the next amino acid at the terminus).

In some embodiments, sequencing of a protein molecule comprises identifying at least two (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more) amino acids in the protein molecule. In some embodiments, the at least two amino acids are contiguous amino acids. In some embodiments, the at least two amino acids are non-contiguous amino acids.

In some embodiments, sequencing of a protein molecule comprises identification of less than 100% (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less) of all amino acids in the protein molecule. For example, in some embodiments, sequencing of a protein molecule comprises identification of less than 100% of one type of amino acid in the protein molecule (e.g., identification of a portion of all amino acids of one type in the protein molecule). In some embodiments, sequencing of a protein molecule comprises identification of less than 100% of each type of amino acid in the protein molecule.

In some embodiments, sequencing of a protein molecule comprises identification of at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 or more types of amino acids in the protein.

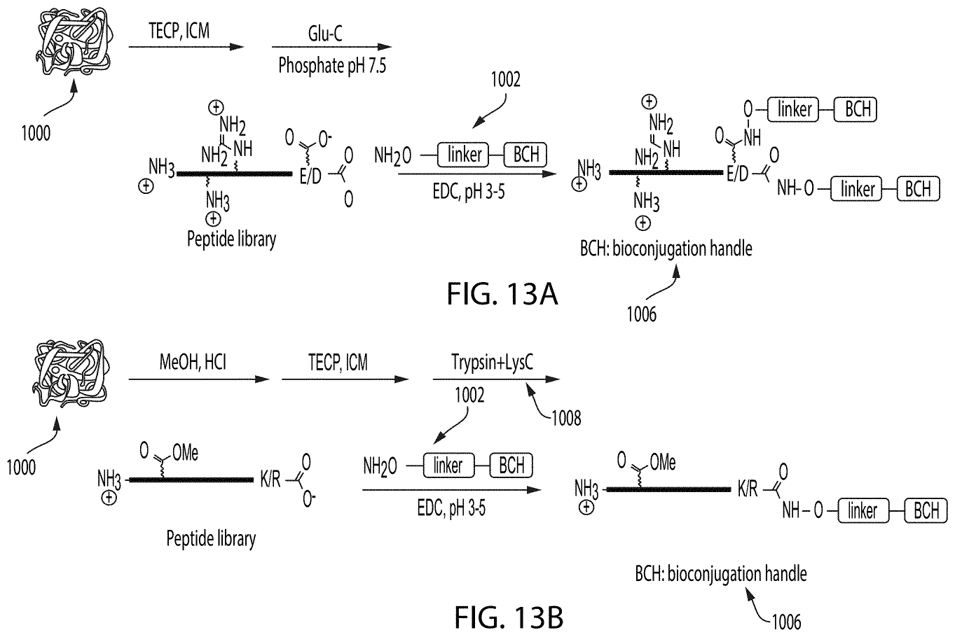

A non-limiting example of protein sequencing by iterative terminal amino acid detection and cleavage is depicted in FIG. 13A. In some embodiments, protein sequencing comprises providing a protein 1000 that is immobilized to a surface 1004 of a solid support (e.g., attached to a bottom or sidewall surface of a sample well) through a linkage group 1002. In some embodiments, linkage group 1002 is formed by a covalent or non-covalent linkage between a functionalized terminal end of protein 1000 and a complementary functional moiety of surface 1004. For example, in some embodiments, linkage group 1002 is formed by a non-covalent linkage between a biotin moiety of protein 1000 (e.g., functionalized in accordance with the disclosure) and an avidin protein of surface 1004. In some embodiments, linkage group 1002 comprises a nucleic acid.

In some embodiments, protein 1000 is immobilized to surface 1004 through a functionalization moiety at one terminal end such that the other terminal end is free for detecting and cleaving of a terminal amino acid in a sequencing reaction. Accordingly, in some embodiments, the reagents used in certain protein sequencing reactions preferentially interact with terminal amino acids at the non-immobilized (e.g., free) terminus of protein 1000. In this way, protein 1000 remains immobilized over repeated cycles of detecting and cleaving. To this end, in some embodiments, linker 1002 may be designed according to a desired set of conditions used for detecting and cleaving, e.g., to limit detachment of protein 1000 from surface 1004. Suitable linker compositions and techniques for functionalizing proteins (e.g., which may be used for immobilizing a protein to a surface) are described in detail elsewhere herein.

In some embodiments, as shown in FIG. 13A, protein sequencing can proceed by (1) contacting protein 1000 with one or more amino acid recognition molecules that associate with one or more types of terminal amino acids. As shown, in some embodiments, a labeled amino acid recognition molecule 1006 interacts with protein 1000 by associating with the terminal amino acid.

In some embodiments, the method further comprises identifying the amino acid (terminal amino acid) of protein 1000 by detecting labeled amino acid recognition molecule 1006. In some embodiments, detecting comprises detecting a luminescence from labeled amino acid recognition molecule 1006. In some embodiments, the luminescence is uniquely associated with labeled amino acid recognition molecule 1006, and the luminescence is thereby associated with the type of amino acid to which labeled amino acid recognition molecule 1006 selectively binds. As such, in some embodiments, the type of amino acid is identified by determining one or more luminescence properties of labeled amino acid recognition molecule 1006.

In some embodiments, protein sequencing proceeds by (2) removing the terminal amino acid by contacting protein 1000 with an exopeptidase 1008 that binds and cleaves the terminal amino acid of protein 1000. Upon removal of the terminal amino acid by exopeptidase 1008, protein sequencing proceeds by (3) subjecting protein 1000 (having n−1 amino acids) to additional cycles of terminal amino acid recognition and cleavage. In some embodiments, steps (1) through (3) occur in the same reaction mixture, e.g., as in a dynamic peptide sequencing reaction. In some embodiments, steps (1) through (3) may be carried out using other methods known in the art, such as peptide sequencing by Edman degradation.

Edman degradation involves repeated cycles of modifying and cleaving the terminal amino acid of a protein, wherein each successively cleaved amino acid is identified to determine an amino acid sequence of the protein. Referring to FIG. 13A, peptide sequencing by conventional Edman degradation can be carried out by (1) contacting protein 1000 with one or more amino acid recognition molecules that selectively bind one or more types of terminal amino acids. In some embodiments, step (1) further comprises removing any of the one or more labeled amino acid recognition molecules that do not selectively bind protein 1000. In some embodiments, step (2) comprises modifying the terminal amino acid (e.g., the free terminal amino acid) of protein 1000 by contacting the terminal amino acid with an isothiocyanate (e.g., PITC) to form an isothiocyanate-modified terminal amino acid. In some embodiments, an isothiocyanate-modified terminal amino acid is more susceptible to removal by a cleaving reagent (e.g., a chemical or enzymatic cleaving reagent) than an unmodified terminal amino acid.

In some embodiments, Edman degradation proceeds by (2) removing the terminal amino acid by contacting protein 1000 with an exopeptidase 1008 that specifically binds and cleaves the isothiocyanate-modified terminal amino acid. In some embodiments, exopeptidase 1008 comprises a modified cysteine protease. In some embodiments, exopeptidase 1008 comprises a modified cysteine protease, such as a cysteine protease from Trypanosoma cruzi (see, e.g., Borgo, et al. (2015) Protein Science 24:571-579). In yet other embodiments, step (2) comprises removing the terminal amino acid by subjecting protein 1000 to chemical (e.g., acidic, basic) conditions sufficient to cleave the isothiocyanate-modified terminal amino acid. In some embodiments, Edman degradation proceeds by (3) washing protein 1000 following terminal amino acid cleavage. In some embodiments, washing comprises removing exopeptidase 1008. In some embodiments, washing comprises restoring protein 1000 to neutral pH conditions (e.g., following chemical cleavage by acidic or basic conditions). In some embodiments, sequencing by Edman degradation comprises repeating steps (1) through (3) for a plurality of cycles.

In some embodiments, peptide sequencing can be carried out in a dynamic peptide sequencing reaction. In some embodiments, referring again to FIG. 13A, the reagents required to perform step (1) and step (2) are combined within a single reaction mixture. For example, in some embodiments, steps (1) and (2) can occur without exchanging one reaction mixture for another and without a washing step as in conventional Edman degradation. Thus, in this embodiments, a single reaction mixture comprises labeled amino acid recognition molecule 1006 and exopeptidase 1008. In some embodiments, exopeptidase 1008 is present in the mixture at a concentration that is less than that of labeled amino acid recognition molecule 1006. In some embodiments, exopeptidase 1008 binds protein 1000 with a binding affinity that is less than that of labeled amino acid recognition molecule 1006.

In some embodiments, dynamic protein sequencing is carried out in real-time by evaluating binding interactions of terminal amino acids with labeled amino acid recognition molecules and a cleaving reagent (e.g., an exopeptidase). FIG. 13B shows an example of a method of sequencing in which discrete binding events give rise to signal pulses of a signal output. The inset panel (left) of FIG. 13B illustrates a general scheme of real-time sequencing by this approach. As shown, a labeled amino acid recognition molecule associates with (e.g., binds to) and dissociates from a terminal amino acid (shown here as phenylalanine), which gives rise to a series of pulses in signal output which may be used to identify the terminal amino acid. In some embodiments, the series of pulses provide a pulsing pattern (e.g., a characteristic pattern) which may be diagnostic of the identity of the corresponding terminal amino acid.

As further shown in the inset panel (left) of FIG. 13B, in some embodiments, a sequencing reaction mixture further comprises an exopeptidase. In some embodiments, the exopeptidase is present in the mixture at a concentration that is less than that of the labeled amino acid recognition molecule. In some embodiments, the exopeptidase displays broad specificity such that it cleaves most or all types of terminal amino acids. Accordingly, a dynamic sequencing approach can involve monitoring recognition molecule binding at a terminus of a protein over the course of a degradation reaction catalyzed by exopeptidase cleavage activity.

FIG. 13B further shows the progress of signal output intensity over time (right panels). In some embodiments, terminal amino acid cleavage by exopeptidase(s) occurs with lower frequency than the binding pulses of a labeled amino acid recognition molecule. In this way, amino acids of a protein may be counted and/or identified in a real-time sequencing process. In some embodiments, one type of amino acid recognition molecule can associate with more than one type of amino acid, where different characteristic patterns correspond to the association of one type of labeled amino acid recognition molecule with different types of terminal amino acids. For example, in some embodiments, different characteristic patterns (as illustrated by each of phenylalanine (F, Phe), tryptophan (W, Trp), and tyrosine (Y, Tyr)) correspond to the association of one type of labeled amino acid recognition molecule (e.g., ClpS protein) with different types of terminal amino acids over the course of degradation. In some embodiments, a plurality of labeled amino acid recognition molecules may be used, each capable of associating with different subsets of amino acids.

In some embodiments, dynamic peptide sequencing is performed by observing different association events, e.g., association events between an amino acid recognition molecule and an amino acid at a terminal end of a peptide, wherein each association event produces a change in magnitude of a signal, e.g., a luminescence signal, that persists for a duration of time. In some embodiments, observing different association events, e.g., association events between an amino acid recognition molecule and an amino acid at a terminal end of a peptide, can be performed during a peptide degradation process. In some embodiments, a transition from one characteristic signal pattern to another is indicative of amino acid cleavage (e.g., amino acid cleavage resulting from peptide degradation). In some embodiments, amino acid cleavage refers to the removal of at least one amino acid from a terminus of a protein (e.g., the removal of at least one terminal amino acid from the protein). In some embodiments, amino acid cleavage is determined by inference based on a time duration between characteristic signal patterns. In some embodiments, amino acid cleavage is determined by detecting a change in signal produced by association of a labeled cleaving reagent with an amino acid at the terminus of the protein. As amino acids are sequentially cleaved from the terminus of the protein during degradation, a series of changes in magnitude, or a series of signal pulses, is detected.

In some embodiments, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, different characteristic patterns corresponding to different types of amino acids in a single protein may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). It should be appreciated that, in some embodiments, smaller differences in mean pulse duration between different characteristic patterns may require a greater number of pulse durations within each characteristic pattern to distinguish one from another with statistical confidence.

Sequencing of proteins in accordance with the instant disclosure, in some aspects, may be performed using a system that permits single molecule analysis. The system may include a sequencing module or device and an instrument configured to interface with the sequencing device. As mentioned above, in some embodiments, detection module 1800 comprises such a sequencing module or device. The sequencing module or device may include an array of pixels, where individual pixels include a sample well and at least one photodetector. The sample wells of the sequencing device may be formed on or through a surface of the sequencing device and be configured to receive a sample placed on the surface of the sequencing device. In some embodiments, the sample wells are a component of a cartridge (e.g., a disposable or single-use cartridge) that can be inserted into the device. Collectively, the sample wells may be considered as an array of sample wells. The plurality of sample wells may have a suitable size and shape such that at least a portion of the sample wells receive a single target molecule or sample comprising a plurality of molecules (e.g. a target protein). In some embodiments, the number of molecules within a sample well may be distributed among the sample wells of the sequencing device such that some sample wells contain one molecule (e.g., a target protein) while others contain zero, two, or a plurality of molecules.

In some embodiments, a sequencing module or device is positioned to receive a target molecule or sample comprising a plurality of molecules (e.g., a target protein) from a sample preparation device. In some embodiments, a sequencing device is connected directly (e.g., physically attached to) or indirectly to a sample preparation device. However, connection between the sample preparation device and the sequencing device or module (or any other type of detection module) is not necessary for all embodiments. In some embodiments, a target molecule (e.g., a target protein) or sample comprising the plurality of molecules is manually transported from the sample preparation device (e.g., sample preparation module) to the sequencing module or device either directly (e.g., without any intervening steps that change the composition of the target molecule or sample) or indirectly (e.g., involving one or more further processing steps that may change the composition of the target molecule or sample). Manual transportation may involve, for example, transport via manual pipetting or suitable manual techniques known in the art.

Excitation light is provided to the sequencing device from one or more light sources external to the sequencing device. Optical components of the sequencing device may receive the excitation light from the light source and direct the light towards the array of sample wells of the sequencing device and illuminate an illumination region within the sample well. In some embodiments, a sample well may have a configuration that allows for the target molecule or sample comprising a plurality of molecules to be retained in proximity to a surface of the sample well, which may ease delivery of excitation light to the sample well and detection of emission light from the target molecule or sample comprising a plurality of molecules. A target molecule or sample comprising a plurality of molecules positioned within the illumination region may emit emission light in response to being illuminated by the excitation light. For example, a protein (or a plurality thereof) may be labeled with a fluorescent marker, which emits light in response to achieving an excited state through the illumination of excitation light. Emission light emitted by a target molecule or sample comprising a plurality of molecules may then be detected by one or more photodetectors within a pixel corresponding to the sample well with the target molecule or sample comprising a plurality of molecules being analyzed. When performed across the array of sample wells, which may range in number between approximately 10,000 pixels to 1,000,000 pixels according to some embodiments, multiple sample wells can be analyzed in parallel.

The sequencing module or device may include an optical system for receiving excitation light and directing the excitation light among the sample well array. The optical system may include one or more grating couplers configured to couple excitation light to the sequencing device and direct the excitation light to other optical components. The optical system may include optical components that direct the excitation light from a grating coupler towards the sample well array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the sequencing device by improving the uniformity of excitation light received by sample wells of the sequencing device. Examples of suitable components, e.g., for coupling excitation light to a sample well and/or directing emission light to a photodetector, to include in a sequencing device are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated herein by reference in their entirety. Examples of suitable grating couplers and waveguides that may be implemented in the sequencing device are described in U.S. patent application Ser. No. 15/844,403, filed Dec. 15, 2017, titled “OPTICAL COUPLER AND WAVEGUIDE SYSTEM,” which is incorporated herein by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the sequencing device, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” which is incorporated herein by reference in its entirety.

Components located off of the sequencing module or device may be used to position and align an excitation source to the sequencing device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated herein by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference in its entirety. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated herein by reference in its entirety.

The photodetector(s) positioned with individual pixels of the sequencing module or device may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the marker associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the sequencing device, and the distribution of arrival times may provide an indication of a timing characteristic of the sample's emission light (e.g., a proxy for luminescence lifetime). In some embodiments, the one or more photodetectors provide an indication of the probability of emission light emitted by the marker (e.g., luminescence intensity). In some embodiments, a plurality of photodetectors may be sized and arranged to capture a spatial distribution of the emission light. Output signals from the one or more photodetectors may then be used to distinguish a marker from among a plurality of markers, where the plurality of markers may be used to identify a sample within the sample. In some embodiments, a sample may be excited by multiple excitation energies, and emission light and/or timing characteristics of the emission light emitted by the sample in response to the multiple excitation energies may distinguish a marker from a plurality of markers.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the sequencing device, which may be connected to an instrument interfaced with the sequencing device. The electrical signals may be subsequently processed and/or analyzed. Processing and/or analyzing of electrical signals may occur on a suitable computing device either located on or off the instrument.

The instrument may include a user interface for controlling operation of the instrument and/or the sequencing device. The user interface may be configured to allow a user to input information into the instrument, such as commands and/or settings used to control the functioning of the instrument. In some embodiments, the user interface may include buttons, switches, dials, and/or a microphone for voice commands. The user interface may allow a user to receive feedback on the performance of the instrument and/or sequencing device, such as proper alignment and/or information obtained by readout signals from the photodetectors on the sequencing device. In some embodiments, the user interface may provide feedback using a speaker to provide audible feedback. In some embodiments, the user interface may include indicator lights and/or a display screen for providing visual feedback to a user.

In some embodiments, the instrument or device described herein may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the sequencing device, and/or data generated from the readout signals of the photodetector.

In some embodiments, the instrument may include a processing device configured to analyze data received from one or more photodetectors of the sequencing device and/or transmit control signals to the excitation source(s). In some embodiments, the processing device may comprise a general purpose processor, and/or a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the sequencing device.

According to some embodiments, the instrument that is configured to analyze target molecules or samples comprising a plurality of molecules based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region may be less complex to operate and maintain, may be more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference in its entirety. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference in its entirety.

In some embodiments, different numbers of fluorophores of the same type may be linked to different components of a target molecule (e.g., a target protein) or a plurality of molecules present in a sample (e.g., a plurality of proteins), so that each individual molecule may be identified based on luminescence intensity. For example, two fluorophores may be linked to a first labeled molecule and four or more fluorophores may be linked to a second labeled molecule. Because of the different numbers of fluorophores, there may be different excitation and fluorophore emission probabilities associated with the different molecule. For example, there may be more emission events for the second labeled molecule during a signal accumulation interval, so that the apparent intensity of the bins is significantly higher than for the first labeled molecule.

The inventors have recognized and appreciated that distinguishing proteins based on fluorophore decay rates and/or fluorophore intensities may facilitate a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation. For example, a limited bandwidth of radiation may include a central or peak wavelength within a 20 nm bandwidth output by a pulsed optical source. In some cases, “characteristic wavelength” or “wavelength” may be used to refer to a peak wavelength within a total bandwidth of radiation output by a source.

In some embodiments, a system comprises a detection module. The detection module (e.g., detection module 1800 in FIG. 9) may be configured to perform any of the variety of abovementioned applications (e.g., bioanalytical applications such as analysis, protein sequencing, peptide sequencing, analyte identification, diagnosis). For example, in some embodiments, the detection module comprises an analysis module. The analysis module may be configured to analyze a sample prepared by the sample preparation module. The analysis module may be configured, for example, to determine a concentration of one or more components in a fluid sample. In some embodiments, the detection module comprises a sequencing module. As an example, referring again to FIG. 9, detection module 1800 comprises a sequencing module, according to some embodiments. The sequencing module may be configured to perform sequencing of one or more components of a sample prepared by the sample preparation module. In some embodiments, the identification module is configured to identify peptide molecules (e.g., protein molecules).

It should be understood that while FIG. 9 depicts shows separate sample preparation module 1700 and detection module 1800 (e.g., analysis module, sequencing module, identification module), the sample preparation module itself (e.g., comprising a peristaltic pump, apparatus, cartridge) may, in some cases, be capable of performing analysis, sequencing, or identification processes. In some embodiments, the sample module is capable of performing a combination of analysis, sequencing, and/or identification processes. For example, in some embodiments, the pump (e.g., pump 1400 that comprises apparatus 1200 and fluidic device 1300) may be configured and/or used to deliver certain volumes (e.g., relatively small volumes, such as less than or equal to 10 μL per pump cycle) of sample (e.g., in sequence and/or at a certain flow rate) directly or indirectly to an integrated detector (e.g., an optical or electrical detector). The integrated detector may be used to make measurements for performing any of a variety of applications (e.g., analysis, sequencing, identification, diagnostics). As such, in certain embodiments, a sample (e.g., comprising a peptide, a protein, bodily tissue, a bodily secretion) prepared by a system described herein can be sequenced/analyzed using any suitable machine (e.g., a different module, or the same module). In certain embodiments, it may be advantageous to have a module described herein for sample preparation and a separate machine for detecting (e.g., sequencing) at least some of (e.g., all of) the samples prepared by the system, e.g., so that the machine may be used with minimal downtime (e.g., continuously) for detection (e.g., sequencing) of samples. In some embodiments, a module for sample preparation (e.g., sample preparation module 1700) may be fluidically connected with a machine (e.g., detection module 1800) for detecting (e.g., sequencing) at least some of (e.g., all of) the samples prepared by the system. In certain embodiments, a system described herein for sample preparation may be fluidically connected with a diagnostic instrument for analyzing at least some of (e.g., all of) the samples prepared by the system. In certain embodiments, the diagnostic instrument generates an output based on the presence or absence of a band or color based on the underlying sequence of a sample. It should be understood that when components (e.g., modules, devices) are described as being connected (e.g., functionally connected), the connections may be permanently connected, or the connections may be reversibly connected. In some instances, components being described as being connected are decoupleably connected, in that they may be connected (e.g., with a fluidic connection via, for example, a channel, tube, conduit) during a first period of time, but then during a second period of time, they may not be connected (e.g., by decoupling the fluidic connection). In some such embodiments, reversible/decoupleable connections may provide for modular systems in which certain components can be replaced or reconfigured, depending on the type of sample preparation/analysis/sequencing/identification being performed.

In another aspect, methods of making a fluidic device (e.g., cartridge) are provided. In some embodiments, a method of making a fluidic device (e.g. cartridge) comprises assembling a surface article comprising a surface layer with a base layer to form the fluidic device (e.g. cartridge), wherein (1) the surface layer comprises an elastomer, (2) the base layer comprises one or more channels, and (3) at least some of the one or more channels have a substantially triangularly-shaped cross-section. Embodiments of methods of making a fluidic device are further described elsewhere herein.

In some embodiments, a method of making a fluidic device (e.g. cartridge) comprises assembling a surface article comprising a surface layer with a base layer to form the fluidic device. In certain embodiments, the surface layer comprises an elastomer. In certain embodiments, the base layer comprises one or more channels. In certain embodiments, at least some of the one or more channels have a substantially triangularly-shaped cross-section.

In certain embodiments, a method comprises manufacturing one or more mechanical components of a fluidic device (e.g. cartridge), e.g., wherein manufacturing comprises injection molding (e.g., precision injection molding). In some embodiments, a method comprises injection molding with hard-steel tooling. In certain embodiments, smooth, defect-free surfaces and tight tolerances (e.g., on the order of tens of microns) are attained for one or more mechanical components manufactured by injection molding with hard-steel tooling, which may be advantageous for manufacturing medical device consumables at high throughput.

In certain embodiments, a method comprises manufacturing one or more components of the fluidic device, such as incubation channels, quenching regions, reservoirs (e.g. derivatization agent reservoirs, derivatization reagent reservoirs) and regions (e.g. incubation region, quenching region, derivatization region, immobilization forming complex region). In some embodiments, manufacturing comprises injection molding (e.g., precision injection molding). In some embodiments, method comprises injection molding with hard steel tooling. In certain embodiments, smooth, defect-free surfaces and tight tolerances (e.g., on the order of tens of microns) are attained for one or more mechanical components manufactured by injection molding with hard-steel tooling, which may be advantageous for manufacturing medical device consumables at high throughput.

In some embodiments, a method comprises over-molding a surface layer comprising an elastomer (e.g., silicone, thermoplastic elastomer) onto a seal plate comprising one or more through-holes (e.g., a hard plastic injection-molded part) to form a surface article comprising the surface layer and the seal plate. In some embodiments, a method comprises assembling a surface article with a base layer to form a fluidic device (e.g. cartridge), wherein assembling comprises, e.g., laser welding, sonic welding, adhering (e.g., using an adhesive), and/or another suitable attachment process for consumables. In certain embodiments, a method comprises aligning the one or more through-holes in the seal plate with corresponding one or more channels in the base layer.

In some embodiments, a method comprises die-cutting (e.g., as an alternative to over-molding) a surface layer comprising an elastomer from pre-made sheet stock, which may advantageously offer high precision in durometer and/or thickness. In some embodiments, a method comprises assembling a surface layer comprising an elastomer (e.g., a die-cut elastomeric layer) between a base layer (e.g., comprising and/or consisting essentially of hard plastic) and a seal plate (e.g., comprising and/or consisting essentially of hard plastic) to form a fluidic device (e.g. cartridge), using, e.g., laser welding, sonic welding, adhering, and/or another suitable attachment process for consumables. In certain embodiments, the base layer comprises one or more channels and the seal plate comprises one or more through-holes. In certain embodiments, a method comprises aligning the one or more through-holes in the seal plate with corresponding one or more channels in the base layer.

In certain embodiments, the surface layer functions as a peristaltic layer, a valve diaphragm, and a face-sealing gasket for the system.

In some embodiments, a method of making a fluidic device (e.g. cartridge) comprises assembling a surface article comprising a surface layer with a base layer to form the fluidic device. In certain embodiments, the surface layer comprises an elastomer. In certain embodiments, the base layer comprises one or more channels. In certain embodiments, at least some of the one or more channels have a substantially triangularly-shaped cross-section.

In some embodiments, assembling the surface article comprising the surface layer with the base layer to form the fluidic device comprises laser welding, sonic welding, and/or adhering the surface layer to the base layer. For example, in some embodiments, a method comprises adhering the surface layer to the base layer using an adhesive.

In some embodiments, a method comprises die-cutting the surface layer comprising the elastomer from pre-made sheet stock. In some embodiments, the surface article consists essentially of the surface layer. In some embodiments, assembling the surface article comprising the surface layer with the base layer to form the fluidic device (e.g. cartridge) comprises assembling the surface layer comprising the elastomer between the base layer and a seal plate to form the fluidic device, wherein the seal plate comprises one or more through-holes. In some embodiments, assembling the surface layer comprising the elastomer between the base layer and the seal plate comprises laser welding, sonic welding, and/or adhering the surface layer to the base layer on one face of the surface layer and to the seal plate on the other face of the surface layer.

In some embodiments, a method comprises over-molding the surface layer comprising the elastomer onto a seal plate comprising one or more through-holes to form the surface article, wherein the surface article further comprises the seal plate.

In some embodiments, at least some of the one or more through-holes of a seal plate have a shape substantially similar to the shape of at least some of the one or more channels of the base layer. In some embodiments, a method comprises aligning one or more through-holes in the seal plate with corresponding one or more channels of the base layer. For example, in certain embodiments, aligning one or more through-holes with one or more channels results in one or more exposed regions of the surface layer, corresponding to one or more exposed regions of the surface layer above one or more associated channels in the base layer, such that a roller (e.g., a roller of an apparatus described herein) may deform an exposed portion of an exposed region of the surface layer to contact a portion of the walls and/or base of an associated channel in the base layer.

In some embodiments, a method comprises injection molding one or more mechanical components of a fluidic device (e.g. cartridge). For example, in certain embodiments, injection molding one or more mechanical components of the fluidic device comprises injection molding to form the seal plate. In certain embodiments, injection molding one or more mechanical components of the fluidic device (e.g. cartridge) comprises injection molding to form the base layer. Injection molding may comprise, for example, precision injection molding and/or injection molding with hard-steel tooling.

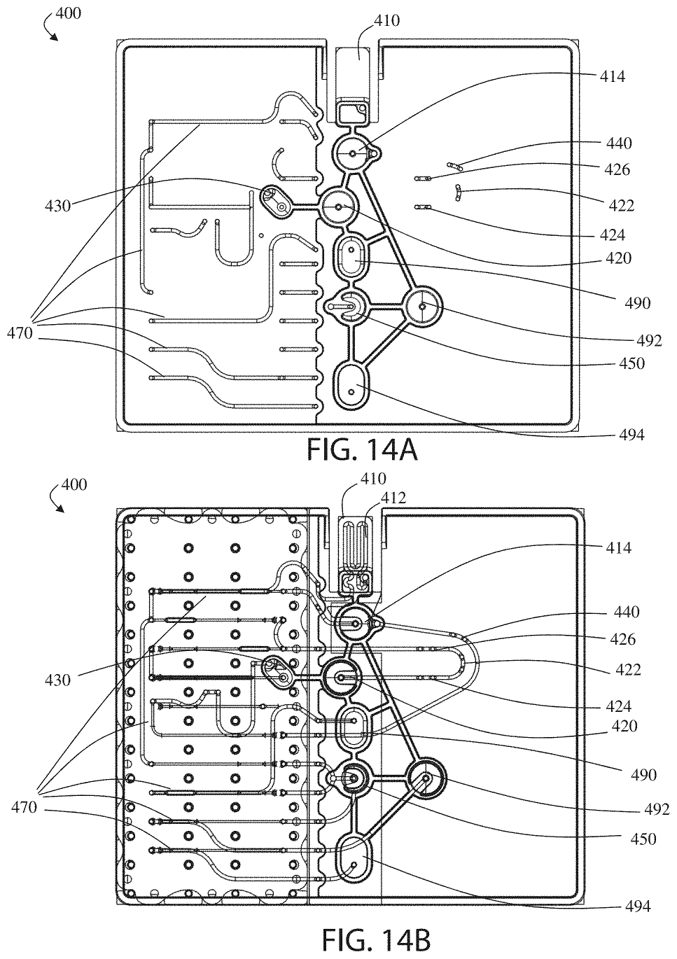

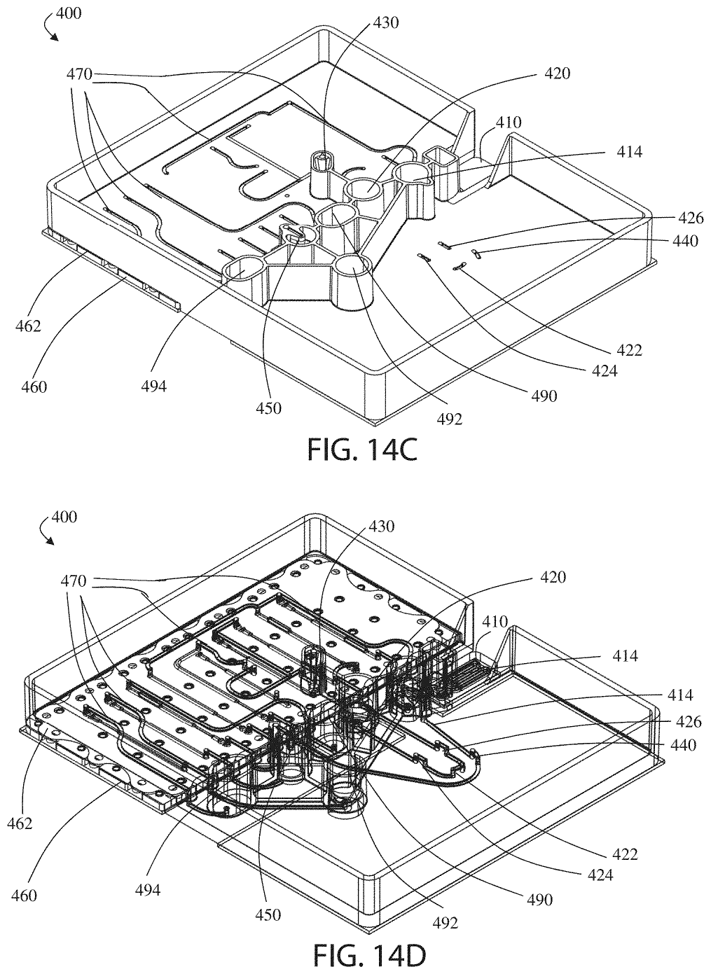

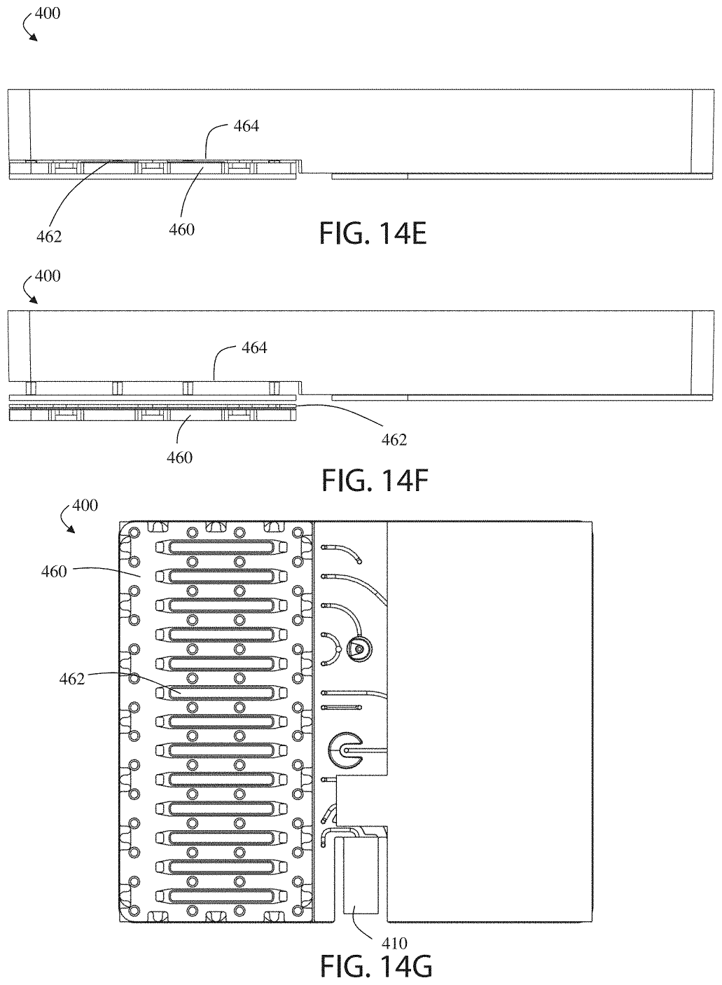

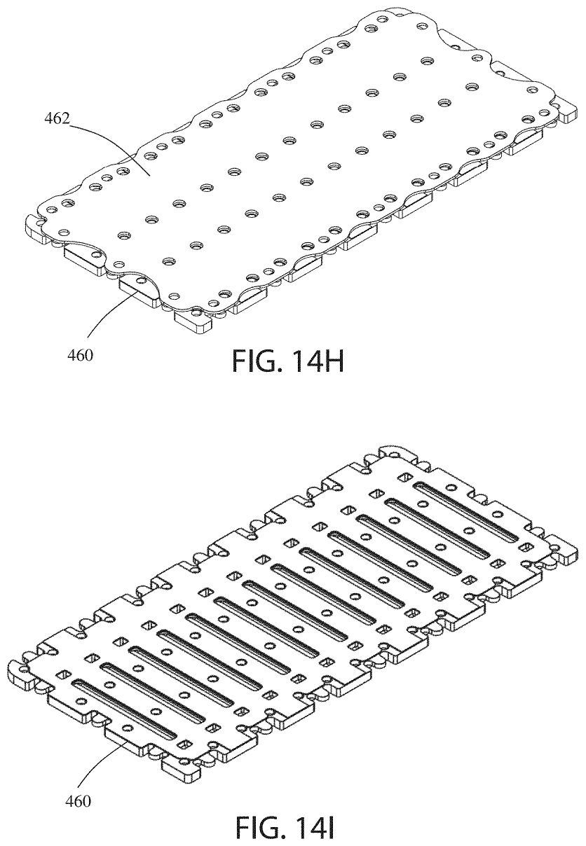

FIGS. 14A-14I show various views of a schematic illustration of a fluidic device for preparing a peptide sample, according to some embodiments. FIG. 14A shows a top-down schematic illustration of fluidic device 400, while FIG. 14B shows a top-down transparency view of fluidic device 400, according to some embodiments. FIG. 14C shows, similarly a perspective schematic illustration of fluidic device 400, while FIG. 14D shows a perspective transparency view of fluidic device 400, according to some embodiments. Fluidic device 400, which is shown in the form of a cartridge, comprises sample loading region 414 fluidically connected to incubation region 410 comprising incubation channel 412 (e.g., via one or more microchannels). Incubation channel 412 may have a serpentine configuration. Sample loading region 414 may be configured to receive a peptide sample (e.g., via a fluidic connection to an external source such as a pipette, syringe, or different fluidic device). Fluidic device 400 further comprises derivatization region 420 fluidically connected to incubation channel 412 via second derivatization reagent reservoir 426, derivatization agent reservoir 422, and first derivatization reagent reservoir 424 via microchannels. Fluidic device 400 further comprises quenching region 430 fluidically connected to derivatization region 420 (e.g., via one or more microchannels). Quenching region 430 may comprise a solid substrate comprising functional groups (e.g., in the form of a plurality of polyamine beads). Quenching region 430 may be configured such that a fluid (e.g., peptide sample) can be recirculated through the quenching region any number of desired times (e.g., 2 times, 3 times, 5 times, 10 times, 20 times, etc.). For example, quenching region 430 may comprise an inlet and an outlet fluidically connected to the inlet. Fluidic device 400 may further comprise immobilization complex-forming region 440 fluidically connected to quenching region 430 (and, in some instances, incubation region 410). Immobilization complex-forming region 440 may comprise or be configured to receive an immobilization complex (e.g., a streptavidin-bearing immobilization complex). In some embodiments, fluidic device 400 comprises purification region 450 (e.g., comprising a size exclusion medium such as a de-salting column) fluidically connected to incubation region 410. Finally, fluidic device may further comprise buffer reservoir 490, collection reservoir 492 (from which purified peptide may be removed from the fluidic device, e.g., for downstream analysis such as sequencing), and waste reservoir 494.

Transportation of fluid and/or reagents within fluidic device 400 may be driven by peristaltic pumping. Fluidic device 400 may be configured for such peristaltic pumping by comprising pumping lanes 470 fluidically collected to some or all of the aforementioned regions and reservoirs of fluidic device 400. Pumping lanes 470 may be channels having a base layer and an elastomer surface. For example, in FIGS. 14C-14D, fluidic device 400 comprises elastomer surface 462 (e.g., a silicone layer) coupled to pumping lanes 470. Interaction with a pumping apparatus (e.g., a roller of an apparatus) may initiate peristaltic action in the pumping lanes, which may actuate fluid transport. Elastomer surface 462 may be fixed to fluidic device 400 via seal plate 460. FIGS. 14E-14G show side view (FIG. 14E), side view exploded (FIG. 14F), and bottom view transparency (FIG. 14G) schematic illustrations of fluidic device 400, showing various views of elastomer surface 462 and seal plate 460. FIGS. 14E and 14F also show base layer 464. FIG. 14H shows a perspective schematic illustration of elastomer surface 462 and seal plate 460, according to some embodiments. FIG. 14I shows a perspective schematic illustration of seal plate 460, according to some embodiments.

Preparation of a peptide sample may, in some embodiments, first comprise lysing and/or enriching a sample (e.g., a biological sample) with respect to a peptide (e.g., a protein). In some embodiments, a peptide sample is formed by making a mixture of a peptide (e.g., a protein), a reducing agent (e.g., TCEP-HCl), an amino acid side chain capping agent (e.g., a cysteine alkylation such as iodoacetamide), and a protein digestion agent (e.g., a protease such as trypsin) in an aqueous buffer (e.g., in 100 mM HEPES or sodium phosphate at pH 8 with 10-20% acetonitrile). The peptide sample may be introduced into sample loading region 414 and transported to incubation channel 410 of incubation region 410. The peptide sample may then be incubated in incubation region 410 (e.g., by maintaining a temperature of 37°). During incubation, the reducing agent may reduce an amino acid side chain (e.g., by reducing a disulfide bond between two cysteine side chains) to denature to the protein. Also during incubation, the amino acid side chain capping agent may form a covalent bond with the reduced amino acid side chain (e.g., by alkylating a resulting cysteine side chain). Also during incubation, the protein digestion agent (e.g., a protease) may induce proteolysis of the protein to form one or more capped peptides, thereby forming a digested protein sample.

The digested protein sample may then be transported through second derivatization reagent reservoir 426 (where it mixes with a pH adjusting reagent such as a base (e.g., K₂CO₃) such that the pH of the sample is 10-11), derivatization agent reservoir 422 (where it mixes with the derivatization agent such as an azide transfer agent), and first derivatization reagent reservoir 424 (where it mixes with a catalyst such as a source of Cu²⁺). The resulting mixture may then be transported to derivatization region 420, where a derivatization reaction may be allowed to occur (e.g., to derivatize one or more side chains such as lysines) to form an unquenched mixture comprising one or more derivatized peptides and excess derivatization agent.

The resulting mixture may then be transferred to quenching region 430, which may comprise a solid substrate (e.g., polyamine beads) that reacts with excess derivatization agent. Recirculation of the sample may occur, and the mixture in the quenching region may be agitated to promote mixing (e.g., via action from the peristaltic pumping components of the fluidic device). The resulting quenched mixture comprising derivatized peptides may undergo a pH adjustment (e.g., to a lower pH such as pH 7-8), such as via exposure to an acid (e.g., acetic acid). The pH-adjusted derivatized peptides may then be transported to immobilization complex-forming region 440, where the derivatized peptides may mix with an immobilization complex such as a streptavidin-bearing immobilization complex (e.g., DBCO-Q24-SV). A mixture of the derivatized peptides and the immobilization complex may be transported to incubation region 410, where an immobilization complex-forming reaction to conjugate the peptides to the immobilization complex may be performed (e.g., by maintaining a temperature of 37° C.). The functionalized peptide sample may then be transported to purification region 450, where any remaining non-functionalized peptides may be removed (e.g., by passing through a size exclusion medium such as a de-salting column integrated into fluidic device 400). The purified peptide sample may then be collected from collection reservoir 492, and waste products (e.g., from the size exclusion medium) may be routed to waste reservoir 494.

Some or all of the steps described above in the context of FIGS. 14A-14I may be performed automatedly.

As used herein, the term “inner surface” regarding a surface layer is used to refer to a surface facing into a channel, whereas an “outer surface” of the surface layer faces an environment outside of the channel. For example, a microchannel may have an inner surface and an outer surface.

As used herein, the terms “first portion” and “second portion” may refer to portions that at least partially overlap or portions having no overlap. For example, a first portion and second portion may substantially overlap.

As used herein, the term “translating” will be known to those of ordinary skill in the art and refers to changing a location. For example, translating may refer to changing a location of a deformation (e.g., elastic deformation).

As used herein, the term “deformation” will be known to those of ordinary skill in the art and refers to a change in shape to an article in response to an applied force. For example, deformation may refer to a change in shape to a surface layer in response to an applied force. Deformation may be elastic. As used herein, the term “elastic deformation” will be known to those of ordinary skill in the art and refers to a temporary change in shape to an article in response to an applied force that is spontaneously reversed upon removal of the applied force. For example, elastic deformation may refer to a temporary change in shape to a surface layer in response to an applied force that is spontaneously reversed upon removal of the applied force.

In some embodiments, components of fluidic devices, articles, and systems described herein are fluidically connected. Two components are fluidically connected if, under some configurations of an embodiment, fluid may pass between them. For example, a first fluidic device component and a second fluidic device component may be in fluidic communication if they are connected by a channel, a microchannel, or a tube. As another example, two components separated by a valve would still be considered fluidically connected, as long as the valve could be configured to permit fluid flow between the two components. In contrast, two components that are only connected mechanically, without a fluidic pathway between them, would not be considered to be fluidically connected. Fluidically connected components may be directly fluidically connected (i.e., connected by a fluidic pathway that does not pass through any intervening components). However, fluidically connected components may in some cases be connected by a fluidic pathway through 1, 2, 3, 4, 5, 8, 10, 15, 20, or more intervening components.

In some embodiments, two compounds are “capable” of reacting with one another. For instance, in some embodiments, a derivatization agent is capable of derivatizing an amino acid side chain. In this context, the word “capable” means that within some temperature range a chemical reaction proceeds spontaneously. For example, two compounds that are capable of reacting with one another may spontaneously chemically react with an amino acid side chain at temperatures greater than or equal to 0° C., the temperature is greater than or equal to 5° C., the temperatures greater than or equal to 10° C., or greater. Two compounds that are capable of reacting with one another may spontaneously chemically react with the amino acid side chain at temperatures less than or equal to 100° C., less than or equal to 80° C., less than or equal to 50° C., less than or equal to 40° C., or less. Combinations of these ranges may be possible. For instance, two compounds that are capable of reacting with one another may spontaneously chemically react with the amino acid side chain at temperatures less than or equal to 100° C. and greater than or equal to 0°.

It should be understood that spontaneous reactions are considered to be spontaneous in the thermodynamic sense, as would be understood by a person of ordinary skill in the art. A spontaneous reaction need not be instantaneous. For example, a spontaneous reaction may take more than 1 minute, more than 5 minutes, more than 10 minutes, more than 1 hour, more than 5 hours, and or more than 24 hours to reach completion. The only requirement of a spontaneous reaction is that progression of the reaction is energetically favorable.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”) In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₀ alkyl (such as substituted C₁₋₆ alkyl, e.g., —CH₂F, —CHF₂, —CF₃ or benzyl (Bn)). An alkyl group may be branched or unbranched.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified

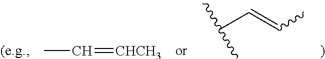

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

Aralkyl” is a subset of “alkyl” and refers to an alkyl group substituted by an aryl group, wherein the point of attachment is on the alkyl moiety

As used herein, the term “alkoxy” refers to an alkyl group having an oxygen atom that connects the alkyl group to the point of attachment: i.e., alkyl-O—. As for the alkyl portions, alkoxy groups can have any suitable number of carbon atoms, such as C₁₋₆ or C₁₋₄. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. Alkoxy groups are unsubstituted, but can be described, in some embodiments as substituted. “Substituted alkoxy” groups can be substituted with one or more moieties selected from halo, hydroxy, amino, alkylamino, nitro, cyano, and alkoxy.

The term “cycloalkyl” refers to cyclic alkyl radical having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

The term “heteroalkyl,” as used herein, refers to an alkyl group, as defined herein, in which one or more of the constituent carbon atoms have been replaced by a heteroatom or optionally substituted heteroatom,

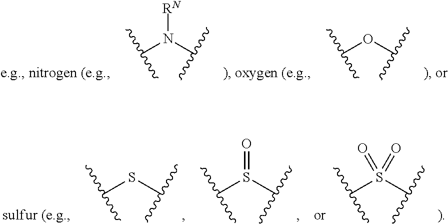

Heteroalkyl groups may be optionally substituted with one, two, three, or, in the case of alkyl groups of two carbons or more, four, five, or six substituents independently selected from any of the substituents described herein. Heteroalkyl group substituents include: (1) carbonyl; (2) halo; (3) C₆-C₁₀ aryl; and (4) C₃-C₁₀ carbocyclyl. A heteroalkylene is a divalent heteroalkyl group.

The term “alkoxy,” as used herein, refers to —OR^{a}, where R^{a} is, e.g., alkyl, alkenyl, alkynyl, aryl, alkylaryl, carbocyclyl, heterocyclyl, or heteroaryl. Examples of alkoxy groups include methoxy, ethoxy, isopropoxy, tert-butoxy, phenoxy, and benzyloxy.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents (e.g., —F, —OH or —O(C₁₋₆ alkyl). In certain embodiments, the aryl group is an unsubstituted C₆₋₁₄ aryl. In certain embodiments, the aryl group is a substituted C₆₋₁₄ aryl.

The term “aryloxy” refers to an —O-aryl substituent.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

The term “amino,” as used herein, represents —N(R^{N})₂, wherein each R^{N} is, independently, H, OH, NO₂, N(R^{N0})₂, SO₂OR^{N0}, SO₂R^{N0}, SOR^{N0}, an N-protecting group, alkyl, alkoxy, aryl, cycloalkyl, acyl (e.g., acetyl, trifluoroacetyl, or others described herein), wherein each of these recited R^{N} groups can be optionally substituted; or two R^{N} combine to form an alkylene or heteroalkylene, and wherein each R^{N0} is, independently, H, alkyl, or aryl. The amino groups of the disclosure can be an unsubstituted amino (i.e., —NH₂) or a substituted amino (i.e., —N(R^{N})₂).

The term “substituted” as used herein means at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, in some embodiments “substituted” means one or more hydrogen atoms are replaced with NR_{g}Rₕ, NR_{g}C(═O)Rₕ, NR_{g}C(═O)NR_{g}Rₕ, NR_{g}C(═O)ORₕ, NR_{g}SO₂Rₕ, OC(═O)NR_{g}Rₕ, OR_{g}, SR_{g}, SOR_{g}, SO₂R_{g}, OSO₂R_{g}, SO₂OR_{g}, ═NSO₂R_{g}, and SO₂NR_{g}Rₕ. “Substituted also means one or more hydrogen atoms are replaced with C(═O)R_{g}, C(═O)OR_{g}, C(═O)NR_{g}Rₕ, CH₂SO₂R_{g}, CH₂SO₂NR_{g}Rₕ.

In the foregoing, R_{g} and Rₕ are the same or different and independently hydrogen, alkyl, alkoxy, alkylaminyl, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means one or more hydrogen atoms are replaced by a bond to an aminyl, cyano, hydroxyl, imino, nitro, oxo, thioxo, halo, alkyl, alkoxy, alkylaminyl, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In addition, each of the foregoing substituents may also be optionally substituted with one or more of the above substituents.

The terms “salt thereof” or “salts thereof” as used herein refer to salts which are well known in the art. For example, Berge et al., describe pharmaceutically acceptable salts in detail in J. Pharmaceutical Sciences, 1977, 66, 1-19, incorporated herein by reference. Additional information on suitable salts can be found in Remington's Pharmaceutical Sciences, 17th ed., Mack Publishing Company, Easton, Pa., 1985, which is incorporated herein by reference. Salts of the compounds of this invention include those derived from suitable inorganic and organic acids and bases. Examples of acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, lower alkyl sulfonate and aryl sulfonate.

A “protein,” “peptide,” or “polypeptide” comprises a polymer of amino acid residues linked together by peptide bonds. The terms refer to proteins, polypeptides, and peptides of any size, structure, or function. Typically, a protein or peptide will be at least three amino acids in length. In some embodiments, a peptide is between about 3 and about 100 amino acids in length (e.g., between about 5 and about 25, between about 10 and about 80, between about 15 and about 70, or between about 20 and about 40, amino acids in length). In some embodiments, a peptide is between about 6 and about 40 amino acids in length (e.g., between about 6 and about 30, between about 10 and about 30, between about 15 and about 40, or between about 20 and about 30, amino acids in length). In some embodiments, a plurality of peptides can refer to a plurality of peptide molecules, where each peptide molecule of the plurality comprises an amino acid sequence that is different from any other peptide molecule of the plurality. In some embodiments, a plurality of peptides can include at least 1 peptide and up to 1,000 peptides (e.g., at least 1 peptide and up to 10, 50, 100, 250, or 500 peptides). In some embodiments, a plurality of peptides comprises 1-5, 5-10, 1-15, 15-20, 10-100, 50-250, 100-500, 500-1,000, or more, different peptides. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein or peptide may be a fragment of a naturally occurring protein or peptide. A protein may be naturally occurring, recombinant, synthetic, or any combination of these.

The following publications are incorporated herein by reference, in their entirety, for all purposes: U.S. Patent Application Publication No. 2021-0121879, published on Apr. 29, 2021, filed as U.S. patent application Ser. No. 17/082,223 on Oct. 28, 2020, and entitled, “Systems and Methods for Sample Preparation”; U.S. Patent Application Publication No. 2021-0164035, published on Jun. 3, 2021, filed as U.S. patent application Ser. No. 17/082,226 on Oct. 28, 2020, and entitled, “Methods and Devices for Sequencing”; U.S. Patent Application Publication No. 2021-0121875, published on Apr. 29, 2021, filed as U.S. patent application Ser. No. 17/083,126 on Oct. 28, 2020, and entitled, “Peristaltic Pumping of Fluids For Bioanalytical Applications and Associated Methods, Systems, and Devices”; and U.S. Patent Application Publication No. 2021-0121874, published on Apr. 29, 2021, filed as U.S. patent application Ser. No. 17/083,106 on Oct. 28, 2020, and entitled, “Peristaltic Pumping of Fluids and Associated Methods, Systems, and Devices.”

U.S. Provisional Patent Application No. 63/139,332, filed Jan. 20, 2021, entitled “Devices and Methods for Peptide Sample Preparation,” is incorporated herein by reference in its entirety for all purposes.

The following examples are intended to illustrate certain embodiments of the present invention, but do not exemplify the full scope of the invention.

### Example 1

This example describes the preparation of a peptides sample using the fluidic device shown in FIGS. 14A-14I, wherein the incubation, derivatization, quenching, immobilization complex forming, and purifying steps were performed on a single fluidic device in the form of a single cartridge. Fluid transportation within the cartridge was initiated via a peristaltic pumping mechanism with a sample preparation module that received the cartridge, as described above. Proteins were prepared by pulldown from spiked plasma, wherein the enriched protein was purified using either an antibody or a DNA aptamer on a solid support. Proteins were then equilibrated with the desired buffer, either by gel filtration or by pH adjustment. Then, an enriched protein sample (50-200 μM in 100 μL) comprising an equal mixture of 2, 3, or 4 proteins was prepared in 100 mM HEPES or sodium phosphate (pH 6-9) with 10-20% acetonitrile was mixed with a solution of tris(2-carboxyethyl)phosphine hydrochloride (TCEP-HCl, 200 mM in water, 1 μL), to act as a reducing agent, freshly dissolved iodoacetamide solution (9 mg in 97.3 μL water for 500 mM, 2 μL), to act as an amino acid side chain capping agent, and Trypsin (1 μg/μL, 0.5-1 μL), to act as a protein digestion agent. Next, the mixture was automatedly transported from a mixture source to an incubation channel (with a serpentine configuration) of an incubation portion of a cartridge. The peptide sample was incubated at 37° C. for 6 to 10 hours in the incubation channel, wherein the protein was denatured and digested. This resulted in the formation of a digested peptide sample.

Next, the digested peptide sample was automatedly transported through a series of reservoirs, where it mixed with a derivatization agent, a first (catalytic) reagent, and a second (pH adjusting) reagent. Initially, the digested peptide sample was automatedly transported to a second derivatization reagent reservoir, where it was added to potassium carbonate (1 M, 5 μL), to adjust the pH to a value of 10-11. Following this, the digested peptide sample was automatedly transferred to a derivatization agent reservoir containing imidazole-1-sulfonyl azide solution (“ISA” 200 mM in 200 mM KOH, 1.2 μL), an azide transfer agent. Next, the digested peptide sample was automatedly transported to a first derivatization reagent reservoir, where it was mixed with copper sulfate (a catalytic reagent) solution. Finally, the digested peptide sample was automatedly transferred to a derivatization region of the fluidic device where was incubated for one hour at room temperature. This resulted in the formation unquenched mixture comprising one or more derivatized peptides.

Following functionalization of the peptides in the derivatization region, 50 μL of the unquenched sample was automatedly transported to a quenching region of the fluidic device. Here, the unquenched mixture was mixed with a plurality of polystyrene beads (a solid substrate), and quenched using 10 actively mixed quench steps, with each quench step followed by a stationary mixing step, for a total of 23 minutes. Finally, the resulting quenched mixture was passed through an on-cartridge column to filter it from the plurality of polystyrene beads.

Next, the pH of the quenched peptide sample was adjusted to between 7 and 8 through the addition of 6 μL of 1 M acetic acid. Following this, the quenched mixture was automatedly mixed with DBCO-Q24-SV (50 μM, 6 μL), an immobilization complex, before being transported back to the incubation channel of the device, where it was incubated at 37° C. for 4 hours. Following this, the peptide sample was automatedly transported to a column of the fluidic device, consisting of Zeba de-salting column resin with a cut off of 40 kDa that was equilibrated first with 10 mM TRIS, 10 mM potassium acetate buffer (pH 7.5). Finally, the purified peptide sample that resulted from this workflow was frozen and stored at a temperature below −20° C.

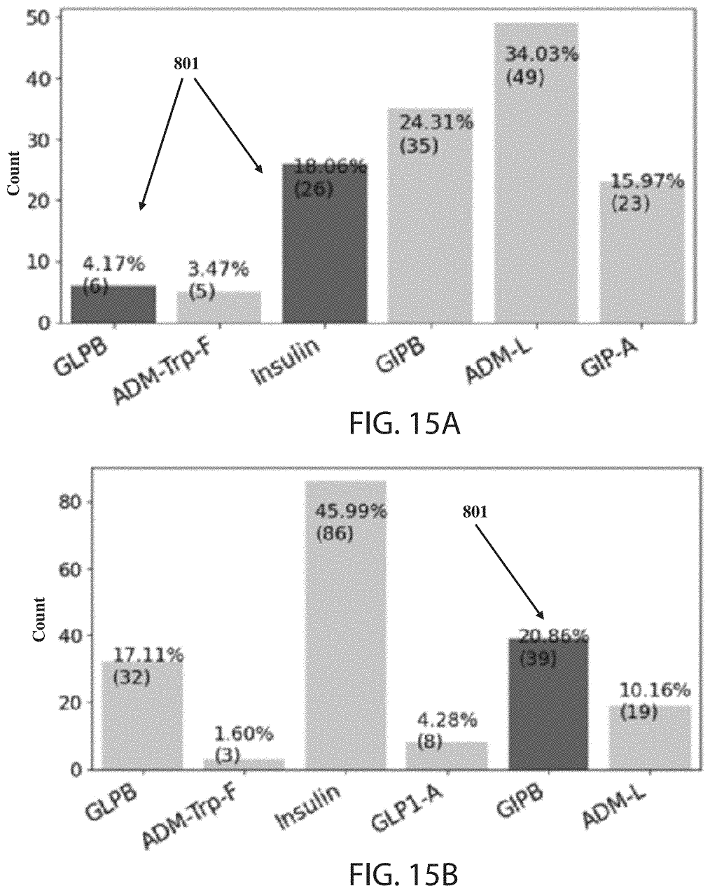

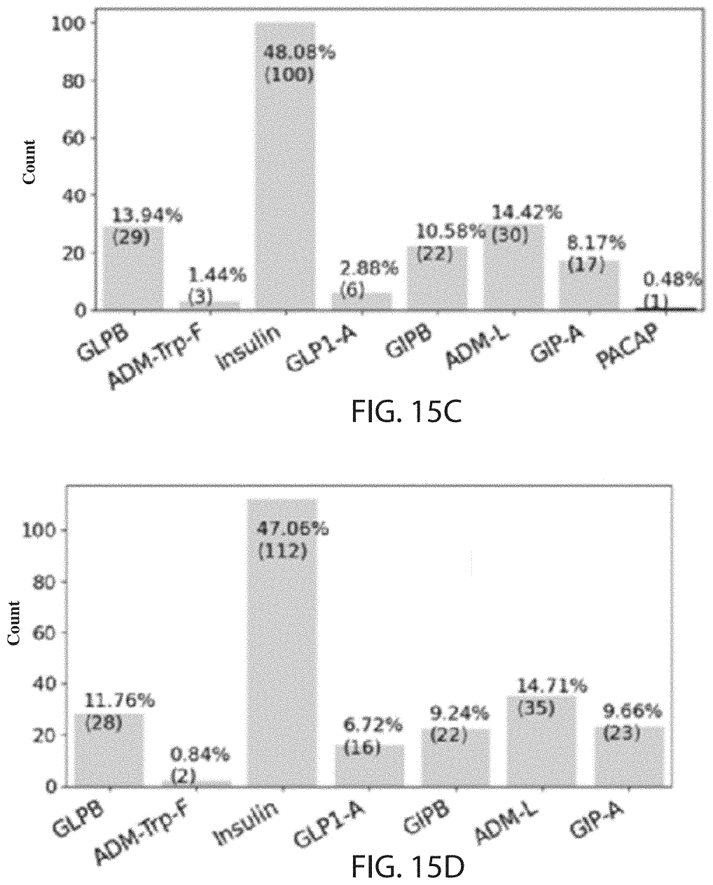

At a later time, purified peptide samples were sequenced, and observed peptides were identified based on their correspondence to protein sequences. FIGS. 15A-15D present the results in the form of bar charts. FIG. 15A corresponds to a mixture of two proteins—GIP and ADM. FIG. 15B corresponds to a mixture of three proteins—GLP1, Insulin, and ADM. FIG. 15C corresponds to a mixture of four proteins—GLP1, ADM, Insulin, and GIP. FIG. 15D corresponds to a mixture of four peptides—GLP1, ADM, Insulin, and GIP. A few off-target assignments 801 are indicated, but in general the peptides sequenced were correctly assigned to the proteins prepared in the peptide sample. Moreover, the generated libraries in this example had similar or more total reads than replicate manually prepared libraries of the same protein mixes. This example demonstrates that a purified peptide sample can be prepared in an automated way on a fluidic device of the type disclosed here.

### Example 2

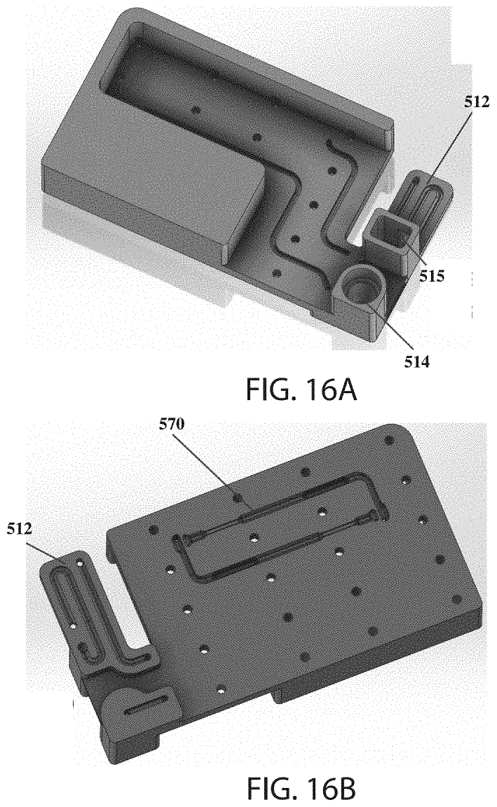

This example describes an exemplary system, wherein the incubation, derivatization, quenching, immobilization complex forming, and purifying steps may be performed using multiple fluidic devices in the form of multiple modular cartridges. Although the fluidic devices of this embodiment are not connected, peptide samples were prepared by following the protocol of Example 1. Fluid transportation within the cartridges was initiated via a peristaltic pumping mechanism with a sample preparation module that received the cartridges, as described above. FIGS. 16A-16B present, respectively, schematic top-view and bottom-view illustrations of the specific embodiment of the first fluidic device used in this example. In the first fluidic device, the protein sample was loaded into mixture source 514. Next, the mixture was automatedly transported from a mixture source to incubation channel 512 (which has a serpentine configuration) of an incubation region of the first fluidic device. The peptide sample was then incubated (e.g. at 37° C. for 5 hours) in the incubation channel, wherein the protein was denatured and digested. The incubation cartridge further comprised pump lanes 570 to facilitate pumping of the fluids within the cartridge, as well as reagent/sample mixture source 514 and evaporation-control water reservoir 515.

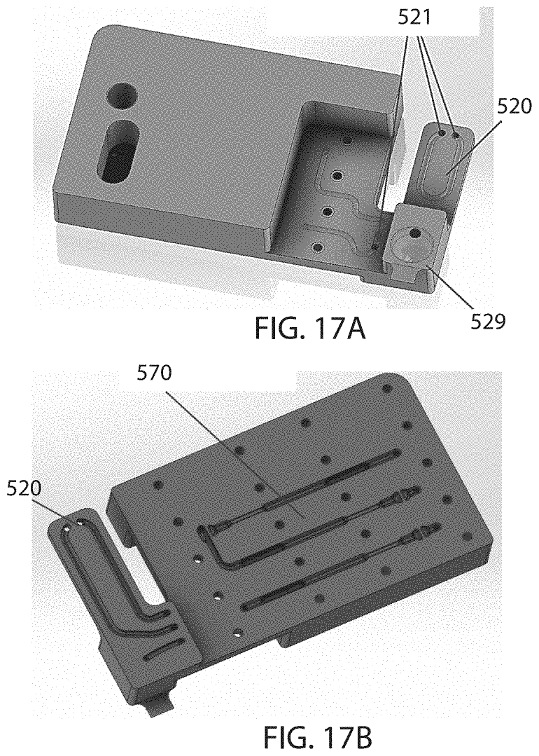

After incubation in this fluidic device portion, the peptide sample became a digested peptide sample. The digested peptide sample was then automatedly transferred to a second fluidic device, where it was automatedly transported through a series of reservoirs, where it mixed with a derivatization agent, a first (catalytic) reagent, and a second (pH adjusting) reagent. FIGS. 17A-17B present, respectively, schematic top-view and bottom-view illustrations of the specific embodiment of the second fluidic device used in this example. The digested peptide sample was transported to the second fluidic device through sample input 529. The digested peptide sample was automatedly transported through the second derivatization reagent reservoir, the derivatization agent reservoir, and the first derivatization reagent reservoir (reservoirs 521 of FIG. 17A), in sequence. Finally, the digested peptide sample was automatedly transferred to channel 520 of a temperature controlled derivatization region of the fluidic device, where it was incubated for the period of time (e.g. one hour at room temperature). This resulted in the formation of an unquenched mixture. The second fluidic device further comprised pump lanes 570.

A portion of the unquenched sample was automatedly transported to a quenching region of a third fluidic device comprising a sample input, a filter for beads, a small volume acidic reagent reservoir, and mixing channels. Here, the unquenched mixture was mixed with a plurality of polystyrene beads (a solid substrate) and lightly agitated in the mixing channels at room temperature. Finally, the resulting quenched mixture was passed through an on-cartridge column to remove the plurality of polystyrene beads, and the pH was adjusted to between 7 and 8 by the addition of acetic acid from an acidic reagent reservoir.

Following this, the quenched mixture was mixed with the DBCO-Q24-SV immobilization complex in the mixture source of the first fluidic device, before it was transported back to the incubation channel of the first fluidic device and incubated at 37° C.

Finally, the peptide sample was automatedly transported to a fourth fluidic device, which controlled the flow of the quenched peptide sample through a commercial Zeba de-salting column resin. Additional equilibration buffer was dispensed through the column to ensure that the peptides were transmitted through the column. The purified peptide sample was collected from a specific fraction of the fluid passing through the column, while the remaining fluid was transmitted to a waste reservoir. The collected purified protein sample was suitable for sequencing using any of the techniques described above. This example demonstrates that in some embodiments, purified peptide samples can be produced automatedly using systems comprising multiple fluidic devices.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A fluidic device for preparing a peptide sample, comprising:
a derivatization agent reservoir configured to receive a derivatization agent capable of derivatizing an amino acid side chain; and
a quenching region fluidically connected to the derivatization agent reservoir via one or more microchannels, wherein the quenching region comprises a solid substrate having a surface comprising functional groups that are capable of reacting with the derivatization agent.

2. A fluidic device for preparing a peptide sample, comprising:
an incubation region configured to facilitate heating of a sample, the incubation region comprising an incubation channel, wherein the incubation channel is a microchannel;
a derivatization region; and
a derivatization agent reservoir configured to receive a derivatization agent capable of derivatizing an amino acid side chain, wherein the derivatization agent reservoir is fluidically connected to the incubation channel and the derivatization region such that a fluid can be transported from the incubation channel, through the derivatization agent reservoir, and to the derivatization region.

3. (canceled)

4. The fluidic device of claim 1, wherein the fluidic device comprises a cartridge.

5. The fluidic device of claim 1, wherein the cartridge comprises a base layer having a surface comprising channels.

6. The fluidic device of claim 5, wherein at least a portion of some of the channels of the cartridge have a surface comprising an elastomer configured to seal off a surface opening of the channel.

7. (canceled)

8. The fluidic device of claim 1, wherein the derivatization agent reservoir comprises the derivatization agent.

9. The fluidic device of claim 1, wherein the derivatization agent comprises an azide transfer agent.

10. The fluidic device of claim 9, wherein the azide transfer agent comprises imidazole-1-sulfonyl azide.

11. (canceled)

12. The fluidic device of claim 1, wherein the solid substrate comprises a bead.

13. The fluidic device of claim 1, wherein the functional groups of the solid substrate comprise amine groups.

14. The fluidic device of claim 1, wherein the quenching region comprises an inlet and an outlet, and the fluidic device is configured such that a fluid can be transported from the outlet of the quenching region to the inlet of the quenching region.

15. The fluidic device of claim 1, wherein the fluidic device comprises an incubation region configured to facilitate heating of a sample, the incubation region comprising an incubation channel.

16. The fluidic device of claim 15, wherein the incubation channel is a microchannel.

17-18. (canceled)

19. The fluidic device of claim 15, wherein the incubation channel is fluidically connected to a source of a mixture comprising a protein, a reducing agent, an amino acid side chain capping agent, and/or a protein digestion agent.

20. The fluidic device of claim 15, wherein the incubation channel is fluidically connected to a source of a mixture comprising a protein, a reducing agent, an amino acid side chain capping agent, and a protein digestion agent.

21. (canceled)

22. The fluidic device of claim 19, wherein the amino acid side chain capping agent comprises a cysteine alkylation agent.

23. (canceled)

24. The fluidic device of claim 19, wherein the protein digestion agent comprises a protease.

25. (canceled)

26. The fluidic device of claim 15, wherein the fluidic device further comprises a derivatization reagent reservoir configured to receive a derivatization reagent capable of facilitating a reaction between the derivatization agent and the amino acid side chain, wherein the derivatization agent reservoir and the derivatization reagent reservoir are fluidically connected such that a fluid can be transported from the incubation channel, through the derivatization agent reservoir and the derivatization reagent reservoir, and to the derivatization region.

27. (canceled)

28. The fluidic device of claim 26, wherein the derivatization reagent comprises a catalyst for a derivatization reaction between the amino acid side chain and the derivatization agent.

29. (canceled)

30. A method for preparing a peptide sample, comprising:
incubating a peptide sample in an incubation region of a fluidic device, the fluidic device comprising at least one microchannel, to form a digested peptide sample, the peptide sample comprising a mixture comprising:
a protein,
a reducing agent,
an amino acid side chain capping agent, and
a protein digestion agent;
wherein during the incubating:
the reducing agent reduces an amino acid side chain of the protein to form a reduced amino acid side chain,
the amino acid side chain capping agent forms a covalent bond with the reduced amino acid side chain to form a capped amino acid side chain, and
the protein digestion agent induces proteolysis of the protein comprising the capped amino acid side chain to form one or more capped peptides, thereby forming the digested peptide sample.

31-76. (canceled)
